(19) United States
(12) Patent Application Publication
(10) Pub. No.: US 2018/0271873 A1
(43) Pub. Date: Sep. 27, 2018

(54) METHODS FOR TREATING VIRAL INFECTIONS

(71) Applicant: The California Institute for Biomedical Research, La Jolla, CA (US)

(72) Inventors: Jason T. ROLAND, San Diego, CA (US); Arnab K. CHATTERJEE, San Diego, CA (US); Peter G. SCHULTZ, La Jolla, CA (US); Shoutian ZHU, San Diego, CA (US); Timothy WRIGHT, Rancho Santa Fe, CA (US); Tyler BAGULEY, San Diego, CA (US)

(21) Appl. No.: 15/562,788

(22) PCT Filed: Mar. 31, 2016
(86) PCT No.: PCT/US16/25364

§ 371 Date: Sep. 28, 2017

Related U.S. Application Data
(60) Provisional application No. 62/141,763, filed on Apr. 1, 2015.

(51) Int. Cl. A61K 31/5377; A61P 31/14; A61K 31/541; A61K 31/506; A61K 31/553; A61K 45/06

(52) U.S. Cl. CPC A61K 31/5377; A61P 31/14; A61K 45/06; A61K 31/506; A61K 31/553; A61K 31/541

(57) ABSTRACT

Described herein are methods for treating a viral infection comprising administering to an individual in need thereof a therapeutically effective amount of a compound of Formula I: Formula (I).

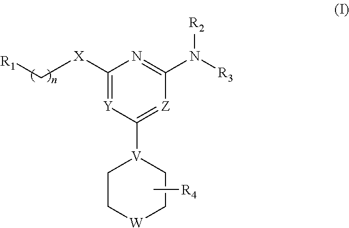

### CROSS-REFERENCE

This application claims benefit of U.S. Provisional Application No. 62/141,763, filed on Apr. 1, 2015, which is herein incorporated by reference in its entirety.

### BACKGROUND OF THE INVENTION

The Ebola virus is one of five known viruses within the genus Ebolavirus. The Ebola virus causes Ebola virus disease (EVD), an acute, serious illness marked by severe bleeding, organ failure and, in many cases, death. EVD first appeared in 1976 and numerous outbreaks of the disease have occurred since then. The 2013-2015 Ebola virus epidemic in West Africa has resulted in nearly 25,000 reported cases of EVD in Guinea, Liberia and Sierra Leone, with over 10,000 reported deaths. Thus, there is a large unmet medical need for new EVD treatments.

### SUMMARY OF THE INVENTION

In one aspect described herein is a method for treating a viral infection comprising administering to an individual in need thereof a therapeutically effective amount of a compound of Formula (I) having the structure:

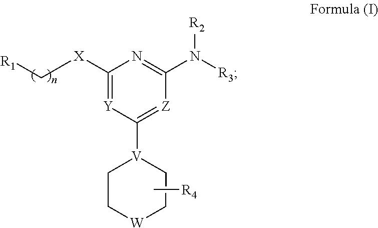

wherein:
X is a bond, —CH₂—, —O—, —S—, —N(R₅)—, —C(═O)—, —S(═O)—, —S(═O)₂—, —C(═N—R₅)—, —C(═N—OR₅)—, or —C(═N—SR₅)—;
one of Y and Z is N, and the other is CR₇;

### V is —C(H)— or —N—;

W is —O—, —S—, —N(R₆)—, —N(R₆)C(═O)—, —S(═O)—, or —S(═O)₂—;
R₁, R₂, R₅, R₆, and R₇ are each independently H, substituted or unsubstituted alkyl, substituted or unsubstituted cycloalkyl, substituted or unsubstituted heterocycloalkyl, substituted or unsubstituted aryl or substituted or unsubstituted heteroaryl;

### R₃ is

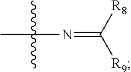

R₄ is H or alkyl;
R₈ and R₉ are each independently H, substituted or unsubstituted alkyl, substituted or unsubstituted aryl, or substituted or unsubstituted heteroaryl; and
n is 0, 1, 2, 3, 4, 5, or 6;
or a pharmaceutically acceptable salt, solvate, or prodrug thereof.

In one embodiment is a method for treating a viral infection comprising administering to an individual in need thereof a therapeutically effective amount of a compound of Formula (I) wherein R₂ is H. In another embodiment is a method for treating a viral infection comprising administering to an individual in need thereof a therapeutically effective amount of a compound of Formula (I) wherein R₄ is H. In another embodiment is a method for treating a viral infection comprising administering to an individual in need thereof a therapeutically effective amount of a compound of Formula (I) wherein R₈ is H. In another embodiment is a method for treating a viral infection comprising administering to an individual in need thereof a therapeutically effective amount of a compound of Formula (I) wherein R₉ is substituted aryl. In another embodiment is a method for treating a viral infection comprising administering to an individual in need thereof a therapeutically effective amount of a compound of Formula (I) wherein R₉ is

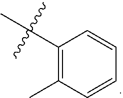 [SMILES: *c1ccccc1C]

In another embodiment is a method for treating a viral infection comprising administering to an individual in need thereof a therapeutically effective amount of a compound of Formula (I) wherein R₉ is

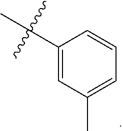 [SMILES: *c1cccc(C)c1]

In another embodiment is a method for treating a viral infection comprising administering to an individual in need thereof a therapeutically effective amount of a compound of Formula (I) wherein R₉ is

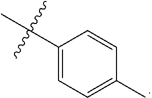 [SMILES: *c1ccc(C)cc1]

In another embodiment is a method for treating a viral infection comprising administering to an individual in need thereof a therapeutically effective amount of a compound of Formula (I) wherein R₈ is H or substituted or unsubstituted alkyl, and R₉ is

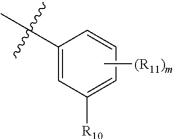

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4. In a further embodiment is a method for treating a viral infection comprising administering to an individual in need thereof a therapeutically effective amount of a compound of Formula (I) wherein R₁₀ is alkyl and m is 0. In yet a further embodiment is a method for treating a viral infection comprising administering to an individual in need thereof a therapeutically effective amount of a compound of Formula (I) wherein R₈ is H. In another embodiment is a method for treating a viral infection comprising administering to an individual in need thereof a therapeutically effective amount of a compound of Formula (I) wherein X is —O—. In another embodiment is a method for treating a viral infection comprising administering to an individual in need thereof a therapeutically effective amount of a compound of Formula (I) wherein Y is N; and Z is CR₇. In a further embodiment is a method for treating a viral infection comprising administering to an individual in need thereof a therapeutically effective amount of a compound of Formula (I) wherein R₇ is H. In another embodiment is a method for treating a viral infection comprising administering to an individual in need thereof a therapeutically effective amount of a compound of Formula (I) wherein V is —N—. In another embodiment is a method for treating a viral infection comprising administering to an individual in need thereof a therapeutically effective amount of a compound of Formula (I) wherein W is —O—. In another embodiment is a method for treating a viral infection comprising administering to an individual in need thereof a therapeutically effective amount of a compound of Formula (I) wherein R₁ is substituted or unsubstituted alkyl, substituted or unsubstituted cycloalkyl, substituted or unsubstituted heterocycloalkyl, substituted or unsubstituted aryl, or substituted or unsubstituted heteroaryl. In another embodiment is a method for treating a viral infection comprising administering to an individual in need thereof a therapeutically effective amount of a compound of Formula (I) wherein R₁ is substituted or unsubstituted heterocycloalkyl, substituted or unsubstituted aryl, or substituted or unsubstituted heteroaryl. In another embodiment is a method for treating a viral infection comprising administering to an individual in need thereof a therapeutically effective amount of a compound of Formula (I) wherein R₁ is substituted or unsubstituted heteroaryl. In another embodiment is a method for treating a viral infection comprising administering to an individual in need thereof a therapeutically effective amount of a compound of Formula (I) wherein R₁ is substituted or unsubstituted quinolinyl. In another embodiment is a method for treating a viral infection comprising administering to an individual in need thereof a therapeutically effective amount of a compound of Formula (I) wherein R₁ is substituted or unsubstituted pyridyl. In another embodiment is a method for treating a viral infection comprising administering to an individual in need thereof a therapeutically effective amount of a compound of Formula (I) wherein R₁ is

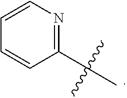 [SMILES: *c1ccccn1]

In another embodiment is a method for treating a viral infection comprising administering to an individual in need thereof a therapeutically effective amount of a compound of Formula (I) wherein n is 0, 1, 2, or 3. In another embodiment is a method for treating a viral infection comprising administering to an individual in need thereof a therapeutically effective amount of a compound of Formula (I) wherein n is 0. In another embodiment is a method for treating a viral infection comprising administering to an individual in need thereof a therapeutically effective amount of a compound of Formula (I) wherein n is 1. In another embodiment is a method for treating a viral infection comprising administering to an individual in need thereof a therapeutically effective amount of a compound of Formula (I) wherein n is 2, 3, 4, 5, or 6. In another embodiment is a method for treating a viral infection comprising administering to an individual in need thereof a therapeutically effective amount of a compound of Formula (I) wherein n is 2.

In some embodiments is a method for treating a viral infection comprising administering to an individual in need thereof a therapeutically effective amount of a compound of Formula (I) having the structure:

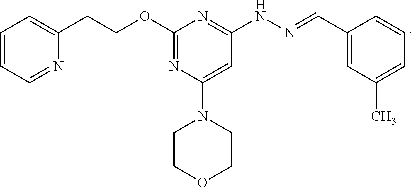 [SMILES: Cc1cccc(/C=N/Nc2cc(N3CCOCC3)nc(OCCc3ccccn3)n2)c1]

In some embodiments is a method for treating a viral infection comprising administering to an individual in need thereof a therapeutically effective amount of a compound of Formula (I) wherein the viral infection is from an RNA virus. In some embodiments is a method for treating a viral infection comprising administering to an individual in need thereof a therapeutically effective amount of a compound of Formula (I) wherein the viral infection is from the Ebola virus.

In some embodiments is a method for treating a viral infection comprising administering to an individual in need thereof a therapeutically effective amount of a compound of Formula (I) further comprising administration of a second agent. In some embodiments is a method for treating a viral infection comprising administering to an individual in need thereof a therapeutically effective amount of a compound of Formula (I) further comprising administration of a second agent wherein the second agent is selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, and T-705. In some embodiments is a method for treating a viral infection comprising administering to an individual in need thereof a therapeutically effective amount of a compound of Formula (I) further comprising administration of a second agent. In some embodiments is a method for treating a viral infection comprising administering to an individual in need thereof a therapeutically effective amount of a compound of Formula (I) further comprising administration of a second agent wherein the second agent is selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, T-705, and GS-5734.

In another aspect described herein is a method for treating a viral infection comprising administering to an individual in need thereof a therapeutically effective amount of a compound of Formula (II) having the structure:

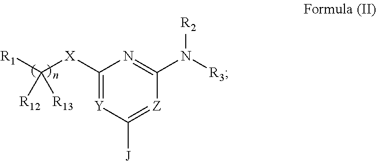

wherein:
X is a bond, —CH₂—, —O—, —S—, —N(R₅)—, —C(═O)—, —S(═O)—, —S(═O)₂—, —C(═N—R₅)—, —C(═N—OR₅)—, or —C(═N—SR₅)—;
one of Y and Z is N, and the other is CR₇;

### J is

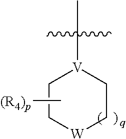

or —N(R₅)(R₆);

### V is —C(H)— or —N—;

W is —O—, —S—, —C(R₆)₂—, —N(R₆)—, —N(R₆)C(═O)—, —S(═O)—, or —S(═O)₂—;
R₁, R₂, R₅, and R₆ are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;

### R₃ is

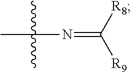

R₄ is substituted or unsubstituted alkyl;
R₇ is H, —CN, halogen, substituted or unsubstituted alkyl, substituted or unsubstituted alkoxy, substituted or unsubstituted cycloalkyl, substituted or unsubstituted heterocycloalkyl, substituted or unsubstituted aryl, or substituted or unsubstituted heteroaryl;
R₈ and R₉ are each independently H, substituted or unsubstituted alkyl, substituted or unsubstituted aryl, or substituted or unsubstituted heteroaryl;
each R₁₂ and R₁₃ are each independently H, halogen, or substituted or unsubstituted alkyl;
n is 0, 1, 2, 3, 4, 5, or 6;
p is 0, 1, 2, 3, 4, 5, or 6; and
q is 1 or 2;
or a pharmaceutically acceptable salt, solvate, or prodrug thereof.

In one embodiment is a method for treating a viral infection comprising administering to an individual in need thereof a therapeutically effective amount of a compound of Formula (II) wherein R₂ is H. In another embodiment is a method for treating a viral infection comprising administering to an individual in need thereof a therapeutically effective amount of a compound of Formula (II) wherein J is

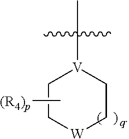

In another embodiment is a method for treating a viral infection comprising administering to an individual in need thereof a therapeutically effective amount of a compound of Formula (II) wherein p is 0 and q is 1. In another embodiment is a method for treating a viral infection comprising administering to an individual in need thereof a therapeutically effective amount of a compound of Formula (II) wherein R₈ is H. In another embodiment is a method for treating a viral infection comprising administering to an individual in need thereof a therapeutically effective amount of a compound of Formula (II) wherein R₉ is substituted aryl. In another embodiment is a method for treating a viral infection comprising administering to an individual in need thereof a therapeutically effective amount of a compound of Formula (II) wherein R₉ is

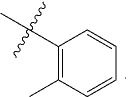 [SMILES: *c1ccccc1C]

In another embodiment is a method for treating a viral infection comprising administering to an individual in need thereof a therapeutically effective amount of a compound of Formula (II) wherein R₉ is

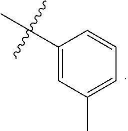 [SMILES: *c1cccc(C)c1]

In another embodiment is a method for treating a viral infection comprising administering to an individual in need thereof a therapeutically effective amount of a compound of Formula (II) wherein R₉ is

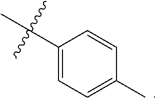 [SMILES: *c1ccc(C)cc1]

In another embodiment is a method for treating a viral infection comprising administering to an individual in need thereof a therapeutically effective amount of a compound of Formula (II) wherein R₈ is H or substituted or unsubstituted alkyl, and R₉ is

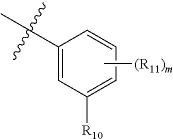

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4. In a further embodiment is a method for treating a viral infection comprising administering to an individual in need thereof a therapeutically effective amount of a compound of Formula (II) wherein R₁₀ is alkyl and m is 0. In yet a further embodiment is a method for treating a viral infection comprising administering to an individual in need thereof a therapeutically effective amount of a compound of Formula (II) wherein R₈ is H. In another embodiment is a method for treating a viral infection comprising administering to an individual in need thereof a therapeutically effective amount of a compound of Formula (II) wherein X is —O—. In another embodiment is a method for treating a viral infection comprising administering to an individual in need thereof a therapeutically effective amount of a compound of Formula (II) wherein Y is N; and Z is CR₇. In a further embodiment is a method for treating a viral infection comprising administering to an individual in need thereof a therapeutically effective amount of a compound of Formula (II) wherein R₇ is H. In another embodiment is a method for treating a viral infection comprising administering to an individual in need thereof a therapeutically effective amount of a compound of Formula (II) wherein V is —N—. In another embodiment is a method for treating a viral infection comprising administering to an individual in need thereof a therapeutically effective amount of a compound of Formula (II) wherein W is —O—. In another embodiment is a method for treating a viral infection comprising administering to an individual in need thereof a therapeutically effective amount of a compound of Formula (II) wherein R₁ is substituted or unsubstituted alkyl, substituted or unsubstituted cycloalkyl, substituted or unsubstituted heterocycloalkyl, substituted or unsubstituted aryl, or substituted or unsubstituted heteroaryl. In another embodiment is a method for treating a viral infection comprising administering to an individual in need thereof a therapeutically effective amount of a compound of Formula (II) wherein R₁ is substituted or unsubstituted heterocycloalkyl, substituted or unsubstituted aryl, or substituted or unsubstituted heteroaryl. In another embodiment is a method for treating a viral infection comprising administering to an individual in need thereof a therapeutically effective amount of a compound of Formula (II) wherein R₁ is substituted or unsubstituted heteroaryl. In another embodiment is a method for treating a viral infection comprising administering to an individual in need thereof a therapeutically effective amount of a compound of Formula (II) wherein R₁ is substituted or unsubstituted quinolinyl. In another embodiment is a method for treating a viral infection comprising administering to an individual in need thereof a therapeutically effective amount of a compound of Formula (II) wherein R₁ is substituted or unsubstituted pyridyl. In another embodiment is a method for treating a viral infection comprising administering to an individual in need thereof a therapeutically effective amount of a compound of Formula (II) wherein R₁ is

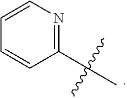 [SMILES: *c1ccccn1]

In another embodiment is a method for treating a viral infection comprising administering to an individual in need thereof a therapeutically effective amount of a compound of Formula (II) wherein each R₁₂ and R₁₃ are H. In another embodiment is a method for treating a viral infection comprising administering to an individual in need thereof a therapeutically effective amount of a compound of Formula (II) wherein n is 0, 1, 2, or 3. In another embodiment is a method for treating a viral infection comprising administering to an individual in need thereof a therapeutically effective amount of a compound of Formula (II) wherein n is 0. In another embodiment is a method for treating a viral infection comprising administering to an individual in need thereof a therapeutically effective amount of a compound of Formula (II) wherein n is 1. In another embodiment is a method for treating a viral infection comprising administering to an individual in need thereof a therapeutically effective amount of a compound of Formula (II) wherein n is 2. In another embodiment is a method for treating a viral infection comprising administering to an individual in need thereof a therapeutically effective amount of a compound of Formula (II) wherein n is 3.

In some embodiments is a method for treating a viral infection comprising administering to an individual in need thereof a therapeutically effective amount of a compound of Formula (II) having the structure:

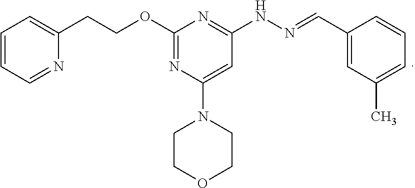 [SMILES: Cc1cccc(/C=N/Nc2cc(N3CCOCC3)nc(OCCc3ccccn3)n2)c1]

In some embodiments is a method for treating a viral infection comprising administering to an individual in need thereof a therapeutically effective amount of a compound of Formula (II) wherein the viral infection is from an RNA virus. In some embodiments is a method for treating a viral infection comprising administering to an individual in need thereof a therapeutically effective amount of a compound of Formula (II) wherein the viral infection is from the Ebola virus.

In some embodiments is a method for treating a viral infection comprising administering to an individual in need thereof a therapeutically effective amount of a compound of Formula (II) further comprising administration of a second agent. In some embodiments is a method for treating a viral infection comprising administering to an individual in need thereof a therapeutically effective amount of a compound of Formula (II) further comprising administration of a second agent wherein the second agent is selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, and T-705. In some embodiments is a method for treating a viral infection comprising administering to an individual in need thereof a therapeutically effective amount of a compound of Formula (II) further comprising administration of a second agent. In some embodiments is a method for treating a viral infection comprising administering to an individual in need thereof a therapeutically effective amount of a compound of Formula (II) further comprising administration of a second agent wherein the second agent is selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, T-705, and GS-5734.

### BRIEF DESCRIPTION OF THE DRAWINGS

FIG. 1 shows a graph of drug induced inhibition of the Ebola virus with kBNC350 (apilimod) versus untreated control.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a straight or branched hydrocarbon chain radical, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —ORₐ where Rₐ is an alkyl radical as defined. Unless stated otherwise specifically in the specification, an alkoxy group may be optionally substituted as described below.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aryloxy” refers to a radical of the formula —ORₐ where Rₐ is an aryl radical as defined. Unless stated otherwise specifically in the specification, an alkoxy group may be optionally substituted as described below.

“Cycloalkyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —ORₐ where Rₐ is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted. The term heterocycloalkyl also includes all ring forms of the carbohydrates, including but not limited to the monosaccharides, the disaccharides and the oligosaccharides.

Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide. Illustrative examples of heteroaryl groups include the following moieties:

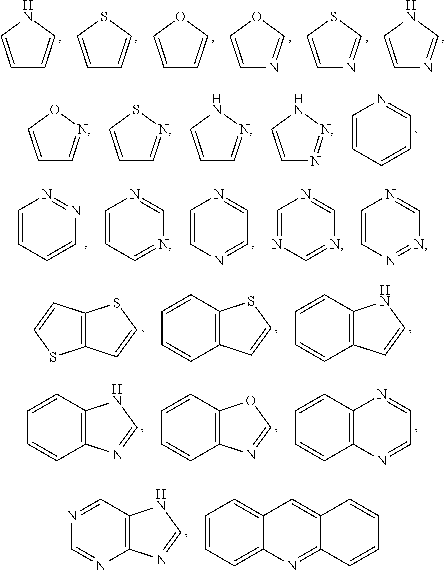

and the like.

The heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl).

“Heteroaryloxy” refers to a radical of the formula —ORₐ where Rₐ is a heteroaryl radical as defined. Unless stated otherwise specifically in the specification, an alkoxy group may be optionally substituted as described below.

All the above groups may be either substituted or unsubstituted. The term “substituted” as used herein means any of the above groups (e.g, alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO₂H, halo, nitrile, nitro, hydroxyl, thiooxy, alkyl, alkylene, alkoxy, aryl, cycloalkyl, heterocyclyl, heteroaryl, dialkylamines, arylamines, alkylarylamines, diarylamines, trialkylammonium (—N⁺R₃), N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NH₂, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarily, an alkyl group may be substituted with one or more internal sulfur atoms to form a thioether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

“Apilimod” or “(E)-4-(6-(2-(3-methylbenzylidene)hydrazinyl)-2-(2-(pyridin-2-yl)ethoxy)pyrimidin-4-yl)morpholine” or “kBNC350” or any other suitable name refers to the compound with the following structure:

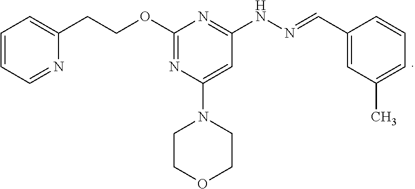 [SMILES: Cc1cccc(/C=N/Nc2cc(N3CCOCC3)nc(OCCc3ccccn3)n2)c1]

Apilimod is also shown as Compound 11 in the Example section.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In other embodiments, treatment also includes prophylactic treatment (e.g., administration of a composition described herein when an individual is suspected to be suffering from a bacterial infection).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

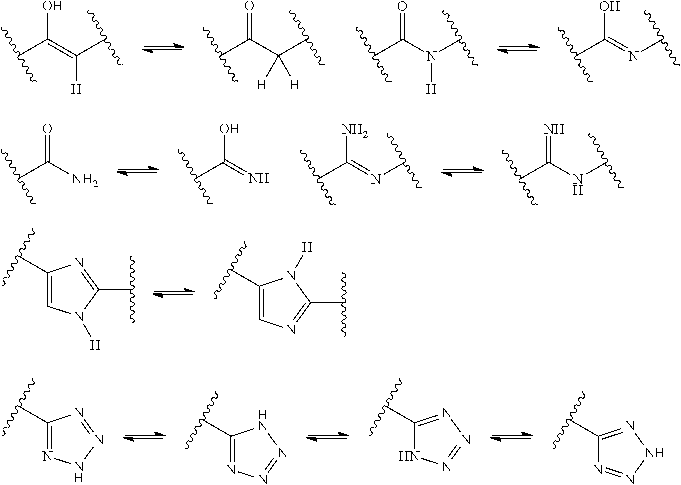

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

### Antiviral Compounds

In the following description of antiviral compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

Described herein are compounds of Formula (I) or (II). Also described herein are pharmaceutically acceptable salts, pharmaceutically acceptable solvates, and pharmaceutically acceptable prodrugs of such compounds. Pharmaceutical compositions that include at least one such compound or a pharmaceutically acceptable salt, pharmaceutically acceptable solvate, or pharmaceutically acceptable prodrug of such compound, are provided. In certain embodiments, isomers and chemically protected forms of compounds having a structure represented by Formula (I) or (II) are also provided.

In one aspect is a method for treating a viral infection comprising administering to an individual in need thereof a therapeutically effective amount of a compound of Formula (I) having the structure:

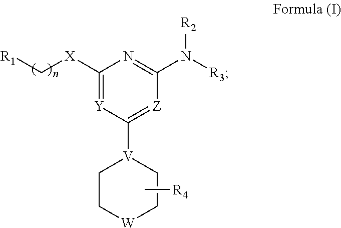

wherein:
X is a bond, —CH₂—, —O—, —S—, —N(R₅)—, —C(═O)—, —S(═O)—, —S(═O)₂—, —C(═N—R₅)—, —C(═N—OR₅)—, or —C(═N—SR₅)—;
one of Y and Z is N, and the other is CR₇;

### V is —C(H)— or —N—;

W is —O—, —S—, —N(R₆)—, —N(R₆)C(═O)—, —S(═O)—, or —S(═O)₂—;
R₁, R₂, R₅, R₆, and R₇ are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;

### R₃ is

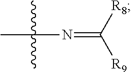

R₄ is H or alkyl;
R₈ and R₉ are each independently H, substituted or unsubstituted alkyl, substituted or unsubstituted aryl, or substituted or unsubstituted heteroaryl; and
n is 0, 1, 2, 3, 4, 5, or 6;
or a pharmaceutically acceptable salt, solvate, or prodrug thereof.

In some embodiments is a method for treating a viral infection comprising administering to an individual in need thereof a therapeutically effective amount of a compound of Formula (I), wherein R₂ is H or substituted or unsubstituted alkyl. In some embodiments is a method for treating a viral infection comprising administering to an individual in need thereof a therapeutically effective amount of a compound of Formula (I), wherein R₂ is H. In some embodiments is a method for treating a viral infection comprising administering to an individual in need thereof a therapeutically effective amount of a compound of Formula (I), wherein R₂ is substituted or unsubstituted alkyl. In some embodiments is a method for treating a viral infection comprising administering to an individual in need thereof a therapeutically effective amount of a compound of Formula (I), wherein R₂ is substituted alkyl. In some embodiments is a method for treating a viral infection comprising administering to an individual in need thereof a therapeutically effective amount of a compound of Formula (I), wherein R₂ is unsubstituted alkyl.

In some embodiments is a method for treating a viral infection comprising administering to an individual in need thereof a therapeutically effective amount of a compound of Formula (I), wherein R₄ is H. In some embodiments is a method for treating a viral infection comprising administering to an individual in need thereof a therapeutically effective amount of a compound of Formula (I), wherein R₄ is alkyl.

In some embodiments is a method for treating a viral infection comprising administering to an individual in need thereof a therapeutically effective amount of a compound of Formula (I), wherein R₈ is H and R₉ is substituted or unsubstituted aryl. In some embodiments is a method for treating a viral infection comprising administering to an individual in need thereof a therapeutically effective amount of a compound of Formula (I), wherein R₈ is H and R₉ is substituted or unsubstituted phenyl. In some embodiments is a method for treating a viral infection comprising administering to an individual in need thereof a therapeutically effective amount of a compound of Formula (I), wherein R₈ is H and R₉ is unsubstituted phenyl. In some embodiments is a method for treating a viral infection comprising administering to an individual in need thereof a therapeutically effective amount of a compound of Formula (I), wherein R₈ is H and R₉ is phenyl substituted with at least one group selected from halogen, —CN, —OH, alkyl, aryl, heteroaryl, alkoxy, aryloxy, or heteroaryloxy. In some embodiments is a method for treating a viral infection comprising administering to an individual in need thereof a therapeutically effective amount of a compound of Formula (I), wherein R₈ is H and R₉ is

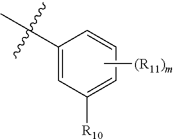

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4. In some embodiments is a method for treating a viral infection comprising administering to an individual in need thereof a therapeutically effective amount of a compound of Formula (I), wherein R₈ is H and R₉ is

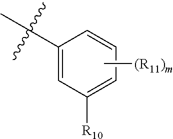

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is H and R₉ is

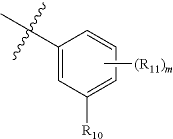

wherein R₁₀ is H, halogen, alkyl, or alkoxy; R₁₁ is halogen or alkyl; and m is 0, 1, or 2. In some embodiments is a method for treating a viral infection comprising administering to an individual in need thereof a therapeutically effective amount of a compound of Formula (I), wherein R₈ is H and R₉ is

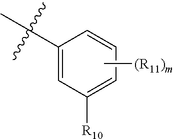

wherein R₁₀ is H; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is H and R₉ is

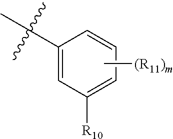

wherein R₁₀ is halogen; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is H and R₉ is

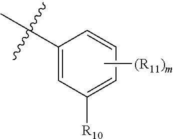

wherein R₁₀ is alkyl; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is H and R₉ is

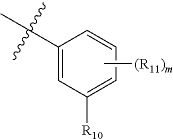

wherein R₁₀ is —CH₃; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is H and R₉ is

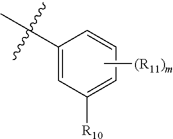

wherein R₁₀ is —CH₃ and m is 0. In some embodiments is a method for treating a viral infection comprising administering to an individual in need thereof a therapeutically effective amount of a compound of Formula (I), wherein R₈ is H and R₉ is

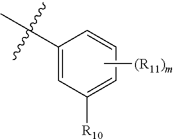

wherein R₁₀ is halogen and m is 0.

In some embodiments is a method for treating a viral infection comprising administering to an individual in need thereof a therapeutically effective amount of a compound of Formula (I), wherein R₈ is substituted or unsubstituted alkyl and R₉ is substituted or unsubstituted aryl.

In some embodiments is a method for treating a viral infection comprising administering to an individual in need thereof a therapeutically effective amount of a compound of Formula (I), wherein R₈ is unsubstituted alkyl and R₉ is substituted or unsubstituted phenyl. In some embodiments is a method for treating a viral infection comprising administering to an individual in need thereof a therapeutically effective amount of a compound of Formula (I), wherein R₈ is unsubstituted alkyl and R₉ is unsubstituted phenyl. In some embodiments is a method for treating a viral infection comprising administering to an individual in need thereof a therapeutically effective amount of a compound of Formula (I), wherein R₈ is unsubstituted alkyl and R₉ is phenyl substituted with at least one group selected from halogen, —CN, —OH, alkyl, aryl, heteroaryl, alkoxy, aryloxy, and heteroaryloxy. In some embodiments is a method for treating a viral infection comprising administering to an individual in need thereof a therapeutically effective amount of a compound of Formula (I), wherein R₈ is unsubstituted alkyl and R₉ is

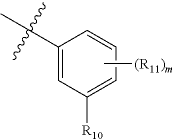

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4. In some embodiments is a method for treating a viral infection comprising administering to an individual in need thereof a therapeutically effective amount of a compound of Formula (I), wherein R₈ is unsubstituted alkyl and R₉ is

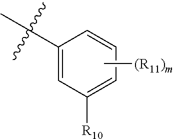

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is unsubstituted alkyl and R₉ is

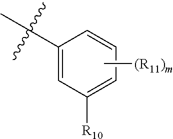

wherein R₁₀ is H, halogen, alkyl, or alkoxy; R₁₁ is halogen or alkyl; and m is 0, 1, or 2. In some embodiments is a method for treating a viral infection comprising administering to an individual in need thereof a therapeutically effective amount of a compound of Formula (I), wherein R₈ is unsubstituted alkyl and R₉ is

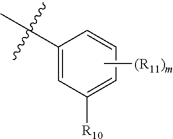

wherein R₁₀ is H; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is unsubstituted alkyl and R₉ is

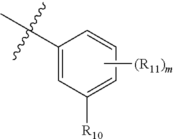

wherein R₁₀ is halogen; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is unsubstituted alkyl and R₉ is

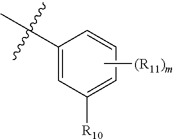

wherein R₁₀ is alkyl; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is unsubstituted alkyl and R₉ is

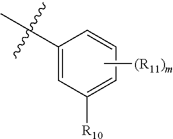

wherein R₁₀ is —CH₃; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (I), wherein R₈ is unsubstituted alkyl and R₉ is

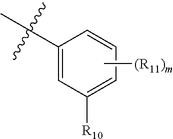

wherein R₁₀ is —CH₃ and m is 0. In some embodiments is a method for treating a viral infection comprising administering to an individual in need thereof a therapeutically effective amount of a compound of Formula (I), wherein R₈ is unsubstituted alkyl and R₉ is

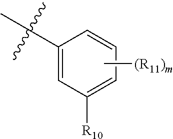

wherein R₁₀ is halogen and m is 0.

In some embodiments is a method for treating a viral infection comprising administering to an individual in need thereof a therapeutically effective amount of a compound of Formula (I), wherein R₈ is H and R₉ is substituted or unsubstituted heteroaryl. In some embodiments is a method for treating a viral infection comprising administering to an individual in need thereof a therapeutically effective amount of a compound of Formula (I), wherein R₈ is H and R₉ is unsubstituted heteroaryl. In some embodiments is a method for treating a viral infection comprising administering to an individual in need thereof a therapeutically effective amount of a compound of Formula (I), wherein R₈ is H and R₉ is heteroaryl substituted with at least one group selected from halogen, —CN, —OH, alkyl, aryl, heteroaryl, alkoxy, aryloxy, and heteroaryloxy.

In some embodiments is a method for treating a viral infection comprising administering to an individual in need thereof a therapeutically effective amount of a compound of Formula (I), wherein R₈ is substituted or unsubstituted alkyl and R₉ is substituted or unsubstituted heteroaryl. In some embodiments is a method for treating a viral infection comprising administering to an individual in need thereof a therapeutically effective amount of a compound of Formula (I), wherein R₈ is substituted alkyl and R₉ is substituted or unsubstituted heteroaryl. In some embodiments is a method for treating a viral infection comprising administering to an individual in need thereof a therapeutically effective amount of a compound of Formula (I), wherein R₈ is unsubstituted alkyl and R₉ is substituted or unsubstituted heteroaryl. In some embodiments is a method for treating a viral infection comprising administering to an individual in need thereof a therapeutically effective amount of a compound of Formula (I), wherein R₈ is unsubstituted alkyl and R₉ is unsubstituted heteroaryl. In some embodiments is a method for treating a viral infection comprising administering to an individual in need thereof a therapeutically effective amount of a compound of Formula (I), wherein R₈ is unsubstituted alkyl and R₉ is heteroaryl substituted with at least one group selected from halogen, —CN, —OH, alkyl, aryl, heteroaryl, alkoxy, aryloxy, and heteroaryloxy.

In any of the aforementioned embodiments is a method for treating a viral infection comprising administering to an individual in need thereof a therapeutically effective amount of a compound of Formula (I), wherein X is —O—. In any of the aforementioned embodiments is a method for treating a viral infection comprising administering to an individual in need thereof a therapeutically effective amount of a compound of Formula (I), wherein X is —S—. In any of the aforementioned embodiments is a method for treating a viral infection comprising administering to an individual in need thereof a therapeutically effective amount of a compound of Formula (I), wherein X is —CH₂—.

In any of the aforementioned embodiments is a method for treating a viral infection comprising administering to an individual in need thereof a therapeutically effective amount of a compound of Formula (I), wherein Y is N and Z is CR₇. In any of the aforementioned embodiments is a method for treating a viral infection comprising administering to an individual in need thereof a therapeutically effective amount of a compound of Formula (I), wherein Y is N and Z is CH.

In any of the aforementioned embodiments is a method for treating a viral infection comprising administering to an individual in need thereof a therapeutically effective amount of a compound of Formula (I), wherein Y is CR₇ and Z is N. In any of the aforementioned embodiments is a method for treating a viral infection comprising administering to an individual in need thereof a therapeutically effective amount of a compound of Formula (I), wherein Y is CH and Z is N.

In any of the aforementioned embodiments is a method for treating a viral infection comprising administering to an individual in need thereof a therapeutically effective amount of a compound of Formula (I), wherein V is —N—. In any of the aforementioned embodiments is a method for treating a viral infection comprising administering to an individual in need thereof a therapeutically effective amount of a compound of Formula (I), wherein V is —C(H)—.

In any of the aforementioned embodiments is a method for treating a viral infection comprising administering to an individual in need thereof a therapeutically effective amount of a compound of Formula (I), wherein W is —O—. In any of the aforementioned embodiments is a method for treating a viral infection comprising administering to an individual in need thereof a therapeutically effective amount of a compound of Formula (I), wherein W is —S—. In any of the aforementioned embodiments is a method for treating a viral infection comprising administering to an individual in need thereof a therapeutically effective amount of a compound of Formula (I), wherein W is —N(R₆)—. In any of the aforementioned embodiments is a method for treating a viral infection comprising administering to an individual in need thereof a therapeutically effective amount of a compound of Formula (I), wherein W is —N(R₆)— and R₆ is H. In any of the aforementioned embodiments is a method for treating a viral infection comprising administering to an individual in need thereof a therapeutically effective amount of a compound of Formula (I), wherein W is —N(R₆)— and R₆ is substituted or unsubstituted alkyl. In any of the aforementioned embodiments is a method for treating a viral infection comprising administering to an individual in need thereof a therapeutically effective amount of a compound of Formula (I), wherein W is —N(R₆)— and R₆ is unsubstituted alkyl.

In any of the aforementioned embodiments is a method for treating a viral infection comprising administering to an individual in need thereof a therapeutically effective amount of a compound of Formula (I), wherein R₁ is substituted or unsubstituted alkyl, substituted or unsubstituted cycloalkyl, substituted or unsubstituted heterocycloalkyl, substituted or unsubstituted aryl, or substituted or unsubstituted heteroaryl. In any of the aforementioned embodiments is a method for treating a viral infection comprising administering to an individual in need thereof a therapeutically effective amount of a compound of Formula (I), wherein R₁ is substituted or unsubstituted heterocycloalkyl, substituted or unsubstituted aryl, or substituted or unsubstituted heteroaryl. In any of the aforementioned embodiments is a method for treating a viral infection comprising administering to an individual in need thereof a therapeutically effective amount of a compound of Formula (I), wherein R₁ is H. In any of the aforementioned embodiments is a method for treating a viral infection comprising administering to an individual in need thereof a therapeutically effective amount of a compound of Formula (I), wherein R₁ is substituted or unsubstituted alkyl. In any of the aforementioned embodiments is a method for treating a viral infection comprising administering to an individual in need thereof a therapeutically effective amount of a compound of Formula (I), wherein R₁ is substituted or unsubstituted cycloalkyl. In any of the aforementioned embodiments is a method for treating a viral infection comprising administering to an individual in need thereof a therapeutically effective amount of a compound of Formula (I), wherein R₁ is substituted or unsubstituted heterocycloalkyl. In any of the aforementioned embodiments is a method for treating a viral infection comprising administering to an individual in need thereof a therapeutically effective amount of a compound of Formula (I), wherein R₁ is substituted or unsubstituted aryl. In any of the aforementioned embodiments is a method for treating a viral infection comprising administering to an individual in need thereof a therapeutically effective amount of a compound of Formula (I), wherein R₁ is substituted or unsubstituted heteroaryl.

In any of the aforementioned embodiments is a method for treating a viral infection comprising administering to an individual in need thereof a therapeutically effective amount of a compound of Formula (I), wherein n is 1, 2, 3, 4, 5, or 6. In any of the aforementioned embodiments is a method for treating a viral infection comprising administering to an individual in need thereof a therapeutically effective amount of a compound of Formula (I), wherein n is 2, 3, 4, 5, or 6. In any of the aforementioned embodiments is a method for treating a viral infection comprising administering to an individual in need thereof a therapeutically effective amount of a compound of Formula (I), wherein n is 0, 1, 2, or 3. In any of the aforementioned embodiments is a method for treating a viral infection comprising administering to an individual in need thereof a therapeutically effective amount of a compound of Formula (I), wherein n is 0. In any of the aforementioned embodiments is a method for treating a viral infection comprising administering to an individual in need thereof a therapeutically effective amount of a compound of Formula (I), wherein n is 1. In any of the aforementioned embodiments is a method for treating a viral infection comprising administering to an individual in need thereof a therapeutically effective amount of a compound of Formula (I), wherein n is 2. In any of the aforementioned embodiments is a method for treating a viral infection comprising administering to an individual in need thereof a therapeutically effective amount of a compound of Formula (I), wherein n is 3. In any of the aforementioned embodiments is a method for treating a viral infection comprising administering to an individual in need thereof a therapeutically effective amount of a compound of Formula (I), wherein n is 4. In any of the aforementioned embodiments is a method for treating a viral infection comprising administering to an individual in need thereof a therapeutically effective amount of a compound of Formula (I), wherein n is 5. In any of the aforementioned embodiments is a method for treating a viral infection comprising administering to an individual in need thereof a therapeutically effective amount of a compound of Formula (I), wherein n is 6. In any of the aforementioned embodiments for treating a viral infection, the viral infection is an RNA virus infection. In any of the aforementioned embodiments for treating a viral infection, the viral infection is an Ebola virus infection.

In some embodiments is a method for treating a viral infection comprising administering to an individual in need thereof a therapeutically effective amount of a compound of Formula (I) having the structure:

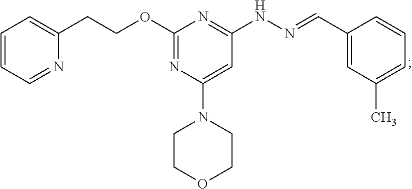 [SMILES: Cc1cccc(/C=N/Nc2cc(N3CCOCC3)nc(OCCc3ccccn3)n2)c1]

or a pharmaceutically acceptable salt, solvate, or prodrug thereof.

In some embodiments is a method for treating a viral infection comprising administering to an individual in need thereof a therapeutically effective amount of a compound of Formula (I) having the structure:

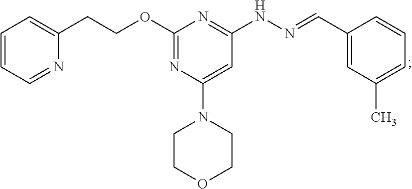 [SMILES: Cc1cccc(/C=N/Nc2cc(N3CCOCC3)nc(OCCc3ccccn3)n2)c1]

or a pharmaceutically acceptable salt, solvate, or prodrug thereof; wherein the viral infection is an Ebola virus infection.

In one aspect is a method for treating a viral infection comprising administering to an individual in need thereof a therapeutically effective amount of a compound of Formula (I) having the structure:

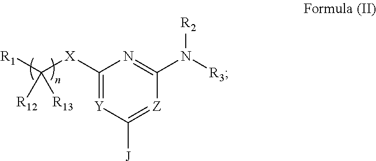

wherein:
X is a bond, —CH₂—, —O—, —S—, —N(R₅)—, —C(═O)—, —S(═O)—, —S(═O)₂—, —C(═N—R₅)—, —C(═N—OR₅)—, or —C(═N—SR₅)—;
one of Y and Z is N, and the other is CR₇;

### J is

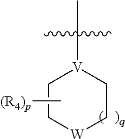

or —N(R₅)(R₆);

### V is —C(H)— or —N—;

W is —O—, —S—, —C(R₆)₂—, —N(R₆)—, —N(R₆)C(═O)—, —S(═O)—, or —S(═O)₂—;
R₁, R₂, R₅, and R₆ are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;

### R₃ is

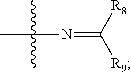

R₄ is substituted or unsubstituted alkyl;
R₇ is H, —CN, halogen, substituted or unsubstituted alkyl, substituted or unsubstituted alkoxy, substituted or unsubstituted cycloalkyl, substituted or unsubstituted heterocycloalkyl, substituted or unsubstituted aryl, or substituted or unsubstituted heteroaryl;
R₈ and R₉ are each independently H, substituted or unsubstituted alkyl, substituted or unsubstituted aryl, or substituted or unsubstituted heteroaryl;
each R₁₂ and R₁₃ are each independently H, halogen, or substituted or unsubstituted alkyl;
n is 0, 1, 2, 3, 4, 5, or 6;
p is 0, 1, 2, 3, 4, 5, or 6; and
q is 1 or 2;
or a pharmaceutically acceptable salt, solvate, or prodrug thereof.

In some embodiments is a method for treating a viral infection comprising administering to an individual in need thereof a therapeutically effective amount of a compound of Formula (II), wherein R₂ is H or substituted or unsubstituted alkyl. In some embodiments is a method for treating a viral infection comprising administering to an individual in need thereof a therapeutically effective amount of a compound of Formula (II), wherein R₂ is H. In some embodiments is a method for treating a viral infection comprising administering to an individual in need thereof a therapeutically effective amount of a compound of Formula (II), wherein R₂ is substituted or unsubstituted alkyl. In some embodiments is a method for treating a viral infection comprising administering to an individual in need thereof a therapeutically effective amount of a compound of Formula (II), wherein R₂ is substituted alkyl. In some embodiments is a method for treating a viral infection comprising administering to an individual in need thereof a therapeutically effective amount of a compound of Formula (II), wherein R₂ is unsubstituted alkyl.

In some embodiments is a method for treating a viral infection comprising administering to an individual in need thereof a therapeutically effective amount of a compound of Formula (II), wherein J is —N(R₅)(R₆).

In some embodiments is a method for treating a viral infection comprising administering to an individual in need thereof a therapeutically effective amount of a compound of Formula (II), wherein J is

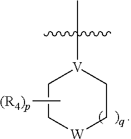

In some embodiments is a method for treating a viral infection comprising administering to an individual in need thereof a therapeutically effective amount of a compound of Formula (II), wherein J is

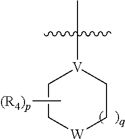

and q is 1. In some embodiments is a method for treating a viral infection comprising administering to an individual in need thereof a therapeutically effective amount of a compound of Formula (II), wherein J is

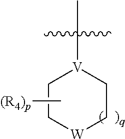

and q is 2.

In some embodiments is a method for treating a viral infection comprising administering to an individual in need thereof a therapeutically effective amount of a compound of Formula (II), wherein J is

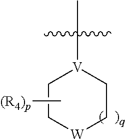

and p is 0. In some embodiments is a method for treating a viral infection comprising administering to an individual in need thereof a therapeutically effective amount of a compound of Formula (II), wherein J is

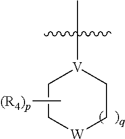

and p is 1. In some embodiments is a method for treating a viral infection comprising administering to an individual in need thereof a therapeutically effective amount of a compound of Formula (II), wherein J is

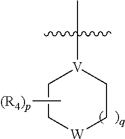

wherein J is and p is 2.

In some embodiments is a method for treating a viral infection comprising administering to an individual in need thereof a therapeutically effective amount of a compound of Formula (II), wherein J is

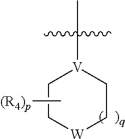

q is 1, and p is 0. In some embodiments is a method for treating a viral infection comprising administering to an individual in need thereof a therapeutically effective amount of a compound of Formula (II), wherein J is

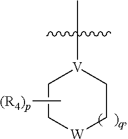

q is 1, and p is 1. In some embodiments is a method for treating a viral infection comprising administering to an individual in need thereof a therapeutically effective amount of a compound of Formula (II), wherein J is

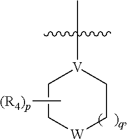

q is 1, and p is 2. In some embodiments is a method for treating a viral infection comprising administering to an individual in need thereof a therapeutically effective amount of a compound of Formula (II), wherein J is

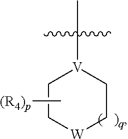

q is 1, p is 2, and R₄ is unsubstituted alkyl. In some embodiments is a method for treating a viral infection comprising administering to an individual in need thereof a therapeutically effective amount of a compound of Formula (II), wherein J is

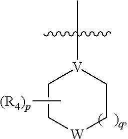

q is 1, p is 2, and R₄ is methyl.

In some embodiments is a method for treating a viral infection comprising administering to an individual in need thereof a therapeutically effective amount of a compound of Formula (II), wherein R₉ is substituted or unsubstituted heteroaryl. In some embodiments is a method for treating a viral infection comprising administering to an individual in need thereof a therapeutically effective amount of a compound of Formula (II), wherein R₉ is substituted or unsubstituted aryl. In some embodiments is a method for treating a viral infection comprising administering to an individual in need thereof a therapeutically effective amount of a compound of Formula (II), wherein R₉ is substituted or unsubstituted phenyl. In some embodiments is a method for treating a viral infection comprising administering to an individual in need thereof a therapeutically effective amount of a compound of Formula (II), wherein R₉ is unsubstituted phenyl. In some embodiments is a method for treating a viral infection comprising administering to an individual in need thereof a therapeutically effective amount of a compound of Formula (II), wherein R₉ is substituted aryl. In some embodiments is a method for treating a viral infection comprising administering to an individual in need thereof a therapeutically effective amount of a compound of Formula (II), wherein R₉ is substituted phenyl. In some embodiments is a method for treating a viral infection comprising administering to an individual in need thereof a therapeutically effective amount of a compound of Formula (II), wherein R₉ is phenyl substituted with at least one group selected from halogen, —CN, —OH, alkyl, aryl, heteroaryl, alkoxy, aryloxy, or heteroaryloxy. In some embodiments is a method for treating a viral infection comprising administering to an individual in need thereof a therapeutically effective amount of a compound of Formula (II), wherein R₉ is

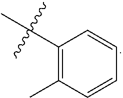 [SMILES: *c1ccccc1C]

In some embodiments is a method for treating a viral infection comprising administering to an individual in need thereof a therapeutically effective amount of a compound of Formula (II), wherein R₉ is

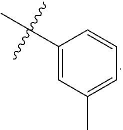 [SMILES: *c1cccc(C)c1]

In some embodiments is a method for treating a viral infection comprising administering to an individual in need thereof a therapeutically effective amount of a compound of Formula (II), wherein R₉ is

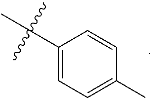 [SMILES: *c1ccc(C)cc1]

In some embodiments is a method for treating a viral infection comprising administering to an individual in need thereof a therapeutically effective amount of a compound of Formula (II), wherein R₈ is H. In some embodiments is a method for treating a viral infection comprising administering to an individual in need thereof a therapeutically effective amount of a compound of Formula (II), wherein R₈ is H and R₉ is substituted or unsubstituted aryl. In some embodiments is a method for treating a viral infection comprising administering to an individual in need thereof a therapeutically effective amount of a compound of Formula (II), wherein R₈ is H and R₉ is substituted aryl. In some embodiments is a method for treating a viral infection comprising administering to an individual in need thereof a therapeutically effective amount of a compound of Formula (II), wherein R₈ is H and R₉ is substituted or unsubstituted phenyl. In some embodiments is a method for treating a viral infection comprising administering to an individual in need thereof a therapeutically effective amount of a compound of Formula (II), wherein R₈ is H and R₉ is unsubstituted phenyl. In some embodiments is a method for treating a viral infection comprising administering to an individual in need thereof a therapeutically effective amount of a compound of Formula (II), wherein R₈ is H and R₉ is substituted phenyl. In some embodiments is a method for treating a viral infection comprising administering to an individual in need thereof a therapeutically effective amount of a compound of Formula (II), wherein R₈ is H and R₉ is phenyl substituted with at least one group selected from halogen, —CN, —OH, alkyl, aryl, heteroaryl, alkoxy, aryloxy, or heteroaryloxy. In some embodiments is a method for treating a viral infection comprising administering to an individual in need thereof a therapeutically effective amount of a compound of Formula (II), wherein R₈ is H and R₉ is

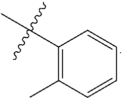 [SMILES: *c1ccccc1C]

In some embodiments is a method for treating a viral infection comprising administering to an individual in need thereof a therapeutically effective amount of a compound of Formula (II), wherein R₈ is H and R₉ is

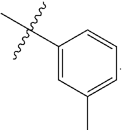 [SMILES: *c1cccc(C)c1]

In some embodiments is a method for treating a viral infection comprising administering to an individual in need thereof a therapeutically effective amount of a compound of Formula (II), wherein R₈ is H and R₉ is

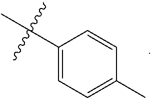 [SMILES: *c1ccc(C)cc1]

In some embodiments is a method for treating a viral infection comprising administering to an individual in need thereof a therapeutically effective amount of a compound of Formula (II), wherein R₈ is H and R₉ is

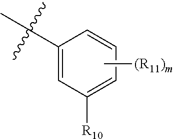

wherein R₁₀ is H, halogen, alkyl, or alkoxy; R₁₁ is halogen, —CN, —OH, alkyl, aryl, heteroaryl, alkoxy, aryloxy, or heteroaryloxy; and
m is 0, 1, 2, 3, or 4. In some embodiments is a method for treating a viral infection comprising administering to an individual in need thereof a therapeutically effective amount of a compound of Formula (II), wherein R₈ is H and R₉ is

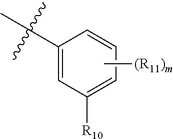

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is H and R₉ is

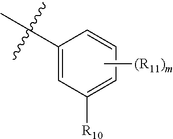

wherein R₁₀ is H, halogen, alkyl, or alkoxy; R₁₁ is halogen or alkyl; and m is 0, 1, or 2. In some embodiments is a method for treating a viral infection comprising administering to an individual in need thereof a therapeutically effective amount of a compound of Formula (II), wherein R₈ is H and R₉ is

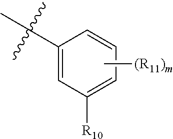

wherein R₁₀ is H; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is H and R₉ is

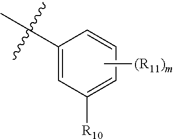

wherein R₁₀ is halogen; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is H and R₉ is

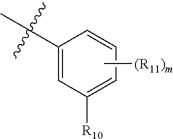

wherein R₁₀ is alkyl; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is H and R₉ is

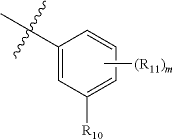

wherein R₁₀ is —CH₃; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is H and R₉ is

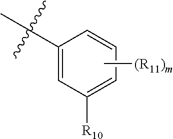

wherein R₁₀ is —CH₃ and m is 0. In some embodiments is a method for treating a viral infection comprising administering to an individual in need thereof a therapeutically effective amount of a compound of Formula (II), wherein R₈ is H and R₉ is

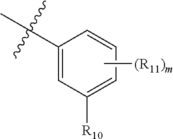

R₁₀ wherein R₁₀ is halogen and m is 0.

In some embodiments is a method for treating a viral infection comprising administering to an individual in need thereof a therapeutically effective amount of a compound of Formula (II), wherein R₈ is substituted or unsubstituted alkyl and R₉ is substituted or unsubstituted aryl.

In some embodiments is a method for treating a viral infection comprising administering to an individual in need thereof a therapeutically effective amount of a compound of Formula (II), wherein R₈ is unsubstituted alkyl and R₉ is substituted or unsubstituted phenyl. In some embodiments is a method for treating a viral infection comprising administering to an individual in need thereof a therapeutically effective amount of a compound of Formula (II), wherein R₈ is unsubstituted alkyl and R₉ is unsubstituted phenyl. In some embodiments is a method for treating a viral infection comprising administering to an individual in need thereof a therapeutically effective amount of a compound of Formula (II), wherein R₈ is unsubstituted alkyl and R₉ is phenyl substituted with at least one group selected from halogen, —CN, —OH, alkyl, aryl, heteroaryl, alkoxy, aryloxy, and heteroaryloxy. In some embodiments is a method for treating a viral infection comprising administering to an individual in need thereof a therapeutically effective amount of a compound of Formula (II), wherein R₈ is unsubstituted alkyl and R₉ is

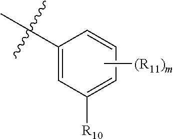

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4. In some embodiments is a method for treating a viral infection comprising administering to an individual in need thereof a therapeutically effective amount of a compound of Formula (II), wherein R₈ is unsubstituted alkyl and R₉ is

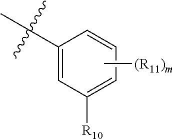

wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, or 2.

In some embodiments is a method for treating a viral infection comprising administering to an individual in need thereof a therapeutically effective amount of a compound of Formula (II), wherein R₈ is unsubstituted alkyl and R₉ is

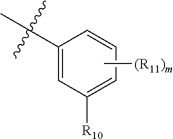

wherein R₁₀ is H, halogen, alkyl, or alkoxy; R₁₁ is halogen or alkyl; and m is 0, 1, or 2. In some embodiments is a method for treating a viral infection comprising administering to an individual in need thereof a therapeutically effective amount of a compound of Formula (II), wherein R₈ is unsubstituted alkyl and R₉ is

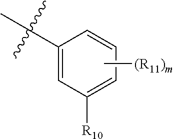

wherein R₁₀ is H; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is unsubstituted alkyl and R₉ is

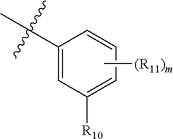

wherein R₁₀ is halogen; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is unsubstituted alkyl and R₉ is

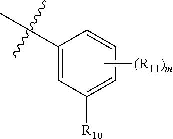

wherein R₁₀ is alkyl; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is unsubstituted alkyl and R₉ is

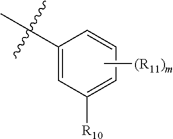

wherein R₁₀ is —CH₃; R₁₁ is halogen, —CN, —OH, alkyl, aryl, heteroaryl, alkoxy, aryloxy, or heteroaryloxy; and m is 0, 1, or 2. In some embodiments is a method for treating a viral infection comprising administering to an individual in need thereof a therapeutically effective amount of a compound of Formula (II), wherein R₈ is unsubstituted alkyl and R₉ is

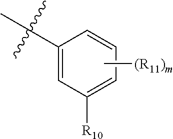

wherein R₁₀ is —CH₃ and m is 0. In some embodiments is a method for treating a viral infection comprising administering to an individual in need thereof a therapeutically effective amount of a compound of Formula (II), wherein R₈ is unsubstituted alkyl and R₉ is

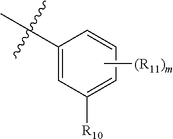

wherein R₁₀ is halogen and m is 0.

In some embodiments is a method for treating a viral infection comprising administering to an individual in need thereof a therapeutically effective amount of a compound of Formula (II), wherein R₈ is H and R₉ is substituted or unsubstituted heteroaryl. In some embodiments is a method for treating a viral infection comprising administering to an individual in need thereof a therapeutically effective amount of a compound of Formula (II), wherein R₈ is H and R₉ is unsubstituted heteroaryl. In some embodiments is a method for treating a viral infection comprising administering to an individual in need thereof a therapeutically effective amount of a compound of Formula (II), wherein R₈ is H and R₉ is heteroaryl substituted with at least one group selected from halogen, —CN, —OH, alkyl, aryl, heteroaryl, alkoxy, aryloxy, and heteroaryloxy.

In some embodiments is a method for treating a viral infection comprising administering to an individual in need thereof a therapeutically effective amount of a compound of Formula (II), wherein R₈ is substituted or unsubstituted alkyl and R₉ is substituted or unsubstituted heteroaryl. In some embodiments is a method for treating a viral infection comprising administering to an individual in need thereof a therapeutically effective amount of a compound of Formula (II), wherein R₈ is substituted alkyl and R₉ is substituted or unsubstituted heteroaryl. In some embodiments is a method for treating a viral infection comprising administering to an individual in need thereof a therapeutically effective amount of a compound of Formula (II), wherein R₈ is unsubstituted alkyl and R₉ is substituted or unsubstituted heteroaryl. In some embodiments is a method for treating a viral infection comprising administering to an individual in need thereof a therapeutically effective amount of a compound of Formula (II), wherein R₈ is unsubstituted alkyl and R₉ is unsubstituted heteroaryl. In some embodiments is a method for treating a viral infection comprising administering to an individual in need thereof a therapeutically effective amount of a compound of Formula (II), wherein R₈ is unsubstituted alkyl and R₉ is heteroaryl substituted with at least one group selected from halogen, —CN, —OH, alkyl, aryl, heteroaryl, alkoxy, aryloxy, and heteroaryloxy.

In any of the aforementioned embodiments is a method for treating a viral infection comprising administering to an individual in need thereof a therapeutically effective amount of a compound of Formula (II), wherein X is —O—. In any of the aforementioned embodiments is a method for treating a viral infection comprising administering to an individual in need thereof a therapeutically effective amount of a compound of Formula (II), wherein X is —S—. In any of the aforementioned embodiments is a method for treating a viral infection comprising administering to an individual in need thereof a therapeutically effective amount of a compound of Formula (II), wherein X is —CH₂—.

In any of the aforementioned embodiments is a method for treating a viral infection comprising administering to an individual in need thereof a therapeutically effective amount of a compound of Formula (II), wherein Y is N and Z is CR₇. In any of the aforementioned embodiments is a method for treating a viral infection comprising administering to an individual in need thereof a therapeutically effective amount of a compound of Formula (II), wherein Y is N, Z is CR₇, and R₇ is H. In any of the aforementioned embodiments is a method for treating a viral infection comprising administering to an individual in need thereof a therapeutically effective amount of a compound of Formula (II), wherein Y is N, Z is CR₇, and R₇ is methyl. In any of the aforementioned embodiments is a method for treating a viral infection comprising administering to an individual in need thereof a therapeutically effective amount of a compound of Formula (II), wherein Y is N, Z is CR₇, and R₇ is methoxy. In any of the aforementioned embodiments is a method for treating a viral infection comprising administering to an individual in need thereof a therapeutically effective amount of a compound of Formula (II), wherein Y is N, Z is CR₇, and R₇ is halogen. In any of the aforementioned embodiments is a method for treating a viral infection comprising administering to an individual in need thereof a therapeutically effective amount of a compound of Formula (II), wherein Y is N, Z is CR₇, and R₇ is —CN.

In any of the aforementioned embodiments is a method for treating a viral infection comprising administering to an individual in need thereof a therapeutically effective amount of a compound of Formula (II), wherein Y is CR₇ and Z is N. In any of the aforementioned embodiments is a method for treating a viral infection comprising administering to an individual in need thereof a therapeutically effective amount of a compound of Formula (II), wherein Z is N, Y is CR₇, and R₇ is H. In any of the aforementioned embodiments is a method for treating a viral infection comprising administering to an individual in need thereof a therapeutically effective amount of a compound of Formula (II), wherein Z is N, Y is CR₇, and R₇ is methyl. In any of the aforementioned embodiments is a method for treating a viral infection comprising administering to an individual in need thereof a therapeutically effective amount of a compound of Formula (II), wherein Z is N, Y is CR₇, and R₇ is methoxy. In any of the aforementioned embodiments is a method for treating a viral infection comprising administering to an individual in need thereof a therapeutically effective amount of a compound of Formula (II), wherein Z is N, Z is CR₇, and R₇ is halogen. In any of the aforementioned embodiments is a method for treating a viral infection comprising administering to an individual in need thereof a therapeutically effective amount of a compound of Formula (II), wherein Y is N, Y is CR₇, and R₇ is —CN.

In any of the aforementioned embodiments is a method for treating a viral infection comprising administering to an individual in need thereof a therapeutically effective amount of a compound of Formula (II), wherein V is —N—. In any of the aforementioned embodiments is a method for treating a viral infection comprising administering to an individual in need thereof a therapeutically effective amount of a compound of Formula (II), wherein V is —C(H)—.

In any of the aforementioned embodiments is a method for treating a viral infection comprising administering to an individual in need thereof a therapeutically effective amount of a compound of Formula (II), wherein W is —O—. In any of the aforementioned embodiments is a method for treating a viral infection comprising administering to an individual in need thereof a therapeutically effective amount of a compound of Formula (II), wherein W is —S—. In any of the aforementioned embodiments is a method for treating a viral infection comprising administering to an individual in need thereof a therapeutically effective amount of a compound of Formula (II), wherein W is —C(R₆)₂—. In any of the aforementioned embodiments is a method for treating a viral infection comprising administering to an individual in need thereof a therapeutically effective amount of a compound of Formula (II), wherein W is —N(R₆)—. In any of the aforementioned embodiments is a method for treating a viral infection comprising administering to an individual in need thereof a therapeutically effective amount of a compound of Formula (II), wherein W is —N(R₆)— and R₆ is H. In any of the aforementioned embodiments is a method for treating a viral infection comprising administering to an individual in need thereof a therapeutically effective amount of a compound of Formula (II), wherein W is —N(R₆)— and R₆ is substituted or unsubstituted alkyl. In any of the aforementioned embodiments is a method for treating a viral infection comprising administering to an individual in need thereof a therapeutically effective amount of a compound of Formula (II), wherein W is —N(R₆)— and R₆ is unsubstituted alkyl.

In any of the aforementioned embodiments is a method for treating a viral infection comprising administering to an individual in need thereof a therapeutically effective amount of a compound of Formula (II), wherein R₁ is substituted or unsubstituted alkyl, substituted or unsubstituted cycloalkyl, substituted or unsubstituted heterocycloalkyl, substituted or unsubstituted aryl, or substituted or unsubstituted heteroaryl. In any of the aforementioned embodiments is a method for treating a viral infection comprising administering to an individual in need thereof a therapeutically effective amount of a compound of Formula (II), wherein R₁ is substituted or unsubstituted heterocycloalkyl, substituted or unsubstituted aryl, or substituted or unsubstituted heteroaryl. In any of the aforementioned embodiments is a method for treating a viral infection comprising administering to an individual in need thereof a therapeutically effective amount of a compound of Formula (II), wherein R₁ is H. In any of the aforementioned embodiments is a method for treating a viral infection comprising administering to an individual in need thereof a therapeutically effective amount of a compound of Formula (II), wherein R₁ is substituted or unsubstituted alkyl. In any of the aforementioned embodiments is a method for treating a viral infection comprising administering to an individual in need thereof a therapeutically effective amount of a compound of Formula (II), wherein R₁ is substituted or unsubstituted cycloalkyl. In any of the aforementioned embodiments is a method for treating a viral infection comprising administering to an individual in need thereof a therapeutically effective amount of a compound of Formula (II), wherein R₁ is substituted or unsubstituted heterocycloalkyl. In any of the aforementioned embodiments is a method for treating a viral infection comprising administering to an individual in need thereof a therapeutically effective amount of a compound of Formula (II), wherein R₁ is substituted or unsubstituted aryl. In any of the aforementioned embodiments is a method for treating a viral infection comprising administering to an individual in need thereof a therapeutically effective amount of a compound of Formula (II), wherein R₁ is substituted or unsubstituted phenyl. In any of the aforementioned embodiments is a method for treating a viral infection comprising administering to an individual in need thereof a therapeutically effective amount of a compound of Formula (II), wherein R₁ is unsubstituted phenyl. In any of the aforementioned embodiments is a method for treating a viral infection comprising administering to an individual in need thereof a therapeutically effective amount of a compound of Formula (II), wherein R₁ is substituted or unsubstituted heteroaryl. In any of the aforementioned embodiments is a method for treating a viral infection comprising administering to an individual in need thereof a therapeutically effective amount of a compound of Formula (II), wherein R₁ is substituted or unsubstituted quinolinyl. In any of the aforementioned embodiments is a method for treating a viral infection comprising administering to an individual in need thereof a therapeutically effective amount of a compound of Formula (II), wherein R₁ is

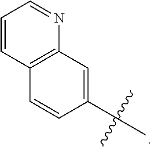 [SMILES: *c1ccc2cccnc2c1]

In any of the aforementioned embodiments is a method for treating a viral infection comprising administering to an individual in need thereof a therapeutically effective amount of a compound of Formula (II), wherein R₁ is

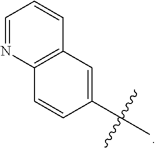 [SMILES: *c1ccc2ncccc2c1]

In any of the aforementioned embodiments is a method for treating a viral infection comprising administering to an individual in need thereof a therapeutically effective amount of a compound of Formula (II), wherein R₁ is

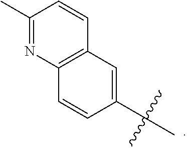 [SMILES: *c1ccc2nc(C)ccc2c1]

In any of the aforementioned embodiments is a method for treating a viral infection comprising administering to an individual in need thereof a therapeutically effective amount of a compound of Formula (II), wherein R₁ is

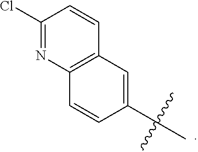 [SMILES: *c1ccc2nc(Cl)ccc2c1]

In any of the aforementioned embodiments is a method for treating a viral infection comprising administering to an individual in need thereof a therapeutically effective amount of a compound of Formula (II), wherein R₁ is

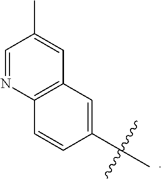 [SMILES: *c1ccc2ncc(C)cc2c1]

In any of the aforementioned embodiments is a method for treating a viral infection comprising administering to an individual in need thereof a therapeutically effective amount of a compound of Formula (II), wherein R₁ is

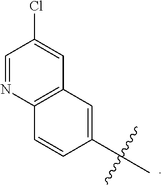 [SMILES: *c1ccc2ncc(Cl)cc2c1]

In any of the aforementioned embodiments is a method for treating a viral infection comprising administering to an individual in need thereof a therapeutically effective amount of a compound of Formula (II), wherein R₁ is

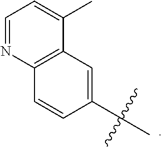 [SMILES: *c1ccc2nccc(C)c2c1]

In any of the aforementioned embodiments is a method for treating a viral infection comprising administering to an individual in need thereof a therapeutically effective amount of a compound of Formula (II), wherein R₁ is

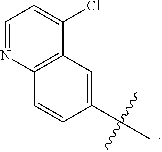 [SMILES: *c1ccc2nccc(Cl)c2c1]

In any of the aforementioned embodiments is a method for treating a viral infection comprising administering to an individual in need thereof a therapeutically effective amount of a compound of Formula (II), wherein R₁ is substituted or unsubstituted pyrimidyl. In any of the aforementioned embodiments is a method for treating a viral infection comprising administering to an individual in need thereof a therapeutically effective amount of a compound of Formula (II), wherein R₁ is

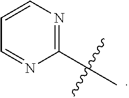 [SMILES: *c1ncccn1]

In any of the aforementioned embodiments is a method for treating a viral infection comprising administering to an individual in need thereof a therapeutically effective amount of a compound of Formula (II), wherein R₁ is

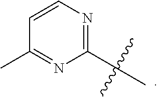 [SMILES: *c1nccc(C)n1]

In any of the aforementioned embodiments is a method for treating a viral infection comprising administering to an individual in need thereof a therapeutically effective amount of a compound of Formula (II), wherein R₁ is

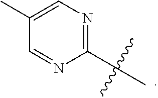 [SMILES: *c1ncc(C)cn1]

In any of the aforementioned embodiments is a method for treating a viral infection comprising administering to an individual in need thereof a therapeutically effective amount of a compound of Formula (II), wherein R₁ is

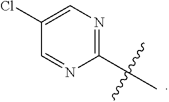 [SMILES: *c1ncc(Cl)cn1]

In any of the aforementioned embodiments is a method for treating a viral infection comprising administering to an individual in need thereof a therapeutically effective amount of a compound of Formula (II), wherein R₁ is substituted or unsubstituted pyridyl. In any of the aforementioned embodiments is a method for treating a viral infection comprising administering to an individual in need thereof a therapeutically effective amount of a compound of Formula (II), wherein R₁ is

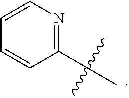 [SMILES: *c1ccccn1]

In any of the aforementioned embodiments is a method for treating a viral infection comprising administering to an individual in need thereof a therapeutically effective amount of a compound of Formula (II), wherein n is 1, 2, 3, 4, 5, or 6. In any of the aforementioned embodiments is a method for treating a viral infection comprising administering to an individual in need thereof a therapeutically effective amount of a compound of Formula (II), wherein n is 2, 3, 4, 5, or 6. In any of the aforementioned embodiments is a method for treating a viral infection comprising administering to an individual in need thereof a therapeutically effective amount of a compound of Formula (II), wherein n is 0, 1, 2, or 3. In any of the aforementioned embodiments is a method for treating a viral infection comprising administering to an individual in need thereof a therapeutically effective amount of a compound of Formula (II), wherein n is 0. In any of the aforementioned embodiments is a method for treating a viral infection comprising administering to an individual in need thereof a therapeutically effective amount of a compound of Formula (II), wherein n is 1. In any of the aforementioned embodiments is a method for treating a viral infection comprising administering to an individual in need thereof a therapeutically effective amount of a compound of Formula (II), wherein n is 2. In any of the aforementioned embodiments is a method for treating a viral infection comprising administering to an individual in need thereof a therapeutically effective amount of a compound of Formula (II), wherein n is 3. In any of the aforementioned embodiments is a method for treating a viral infection comprising administering to an individual in need thereof a therapeutically effective amount of a compound of Formula (II), wherein n is 4. In any of the aforementioned embodiments is a method for treating a viral infection comprising administering to an individual in need thereof a therapeutically effective amount of a compound of Formula (II), wherein n is 5. In any of the aforementioned embodiments is a method for treating a viral infection comprising administering to an individual in need thereof a therapeutically effective amount of a compound of Formula (II), wherein n is 6.

In any of the aforementioned embodiments is a method for treating a viral infection comprising administering to an individual in need thereof a therapeutically effective amount of a compound of Formula (II), wherein each R₁₂ and R₁₃ are each independently H, halogen, or unsubstituted alkyl. In any of the aforementioned embodiments is a method for treating a viral infection comprising administering to an individual in need thereof a therapeutically effective amount of a compound of Formula (II), wherein each R₁₂ and R₁₃ are H.

In any of the aforementioned embodiments is a method for treating a viral infection comprising administering to an individual in need thereof a therapeutically effective amount of a compound of Formula (II), wherein q is 1. In any of the aforementioned embodiments is a method for treating a viral infection comprising administering to an individual in need thereof a therapeutically effective amount of a compound of Formula (II), wherein q is 2. In any of the aforementioned embodiments for treating a viral infection, the viral infection is an RNA virus infection. In any of the aforementioned embodiments for treating a viral infection, the viral infection is an Ebola virus infection.

In some embodiments is a method for treating a viral infection comprising administering to an individual in need thereof a therapeutically effective amount of a compound of Formula (II) having the structure:

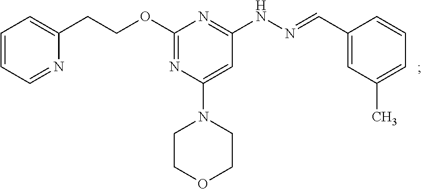 [SMILES: Cc1cccc(/C=N/Nc2cc(N3CCOCC3)nc(OCCc3ccccn3)n2)c1]

or a pharmaceutically acceptable salt, solvate, or prodrug thereof.

In some embodiments is a method for treating a viral infection comprising administering to an individual in need thereof a therapeutically effective amount of a compound of Formula (II) having the structure:

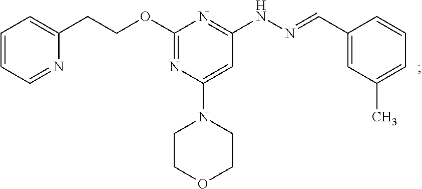 [SMILES: Cc1cccc(/C=N/Nc2cc(N3CCOCC3)nc(OCCc3ccccn3)n2)c1]

or a pharmaceutically acceptable salt, solvate, or prodrug thereof; wherein the viral infection is an Ebola virus infection.

In some embodiments is a compound having the structure:

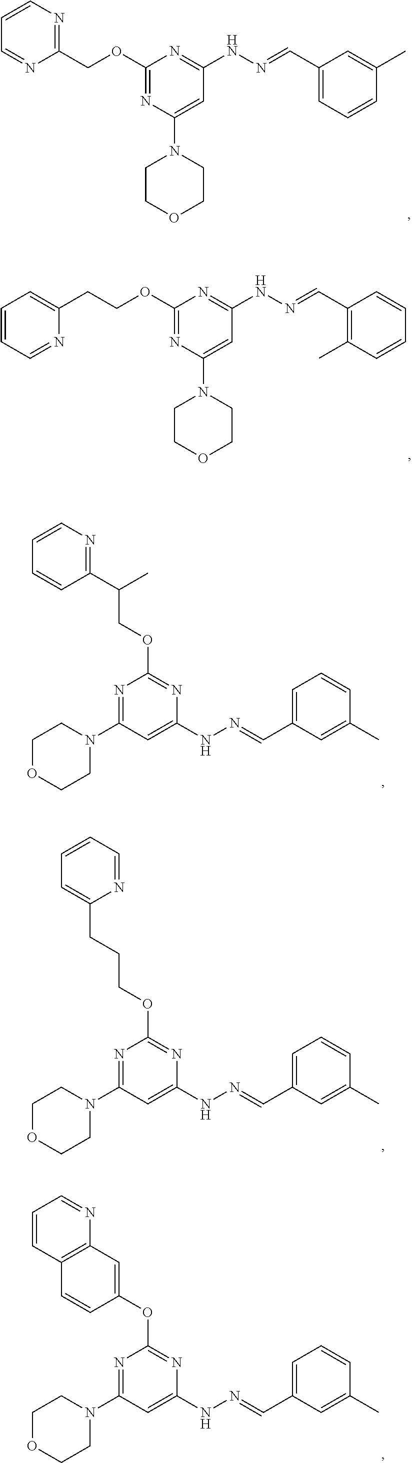

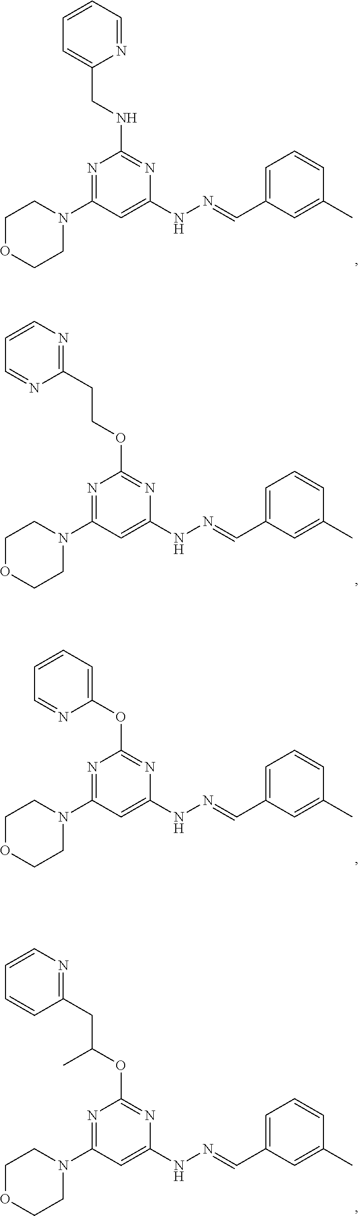 [SMILES: Cc1cccc(/C=N/Nc2cc(N3CCOCC3)nc(NCc3ccccn3)n2)c1 ; Cc1cccc(/C=N/Nc2cc(N3CCOCC3)nc(OCCc3ncccn3)n2)c1 ; Cc1cccc(/C=N/Nc2cc(N3CCOCC3)nc(Oc3ccccn3)n2)c1 ; Cc1cccc(/C=N/Nc2cc(N3CCOCC3)nc(OC(C)Cc3ccccn3)n2)c1]

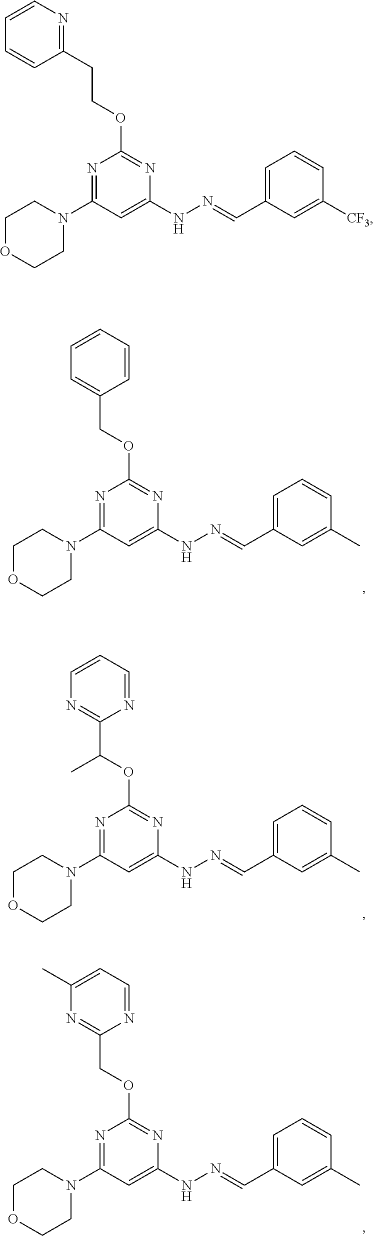

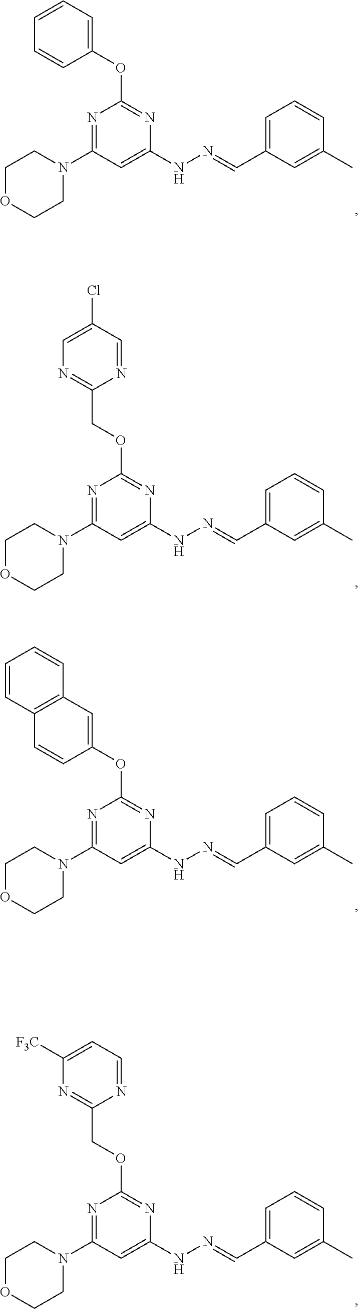 [SMILES: Cc1cccc(/C=N/Nc2cc(N3CCOCC3)nc(Oc3ccccc3)n2)c1 ; Cc1cccc(/C=N/Nc2cc(N3CCOCC3)nc(OCc3ncc(Cl)cn3)n2)c1 ; Cc1cccc(/C=N/Nc2cc(N3CCOCC3)nc(Oc3ccc4ccccc4c3)n2)c1 ; Cc1cccc(/C=N/Nc2cc(N3CCOCC3)nc(OCc3nccc(C(F)(F)F)n3)n2)c1]

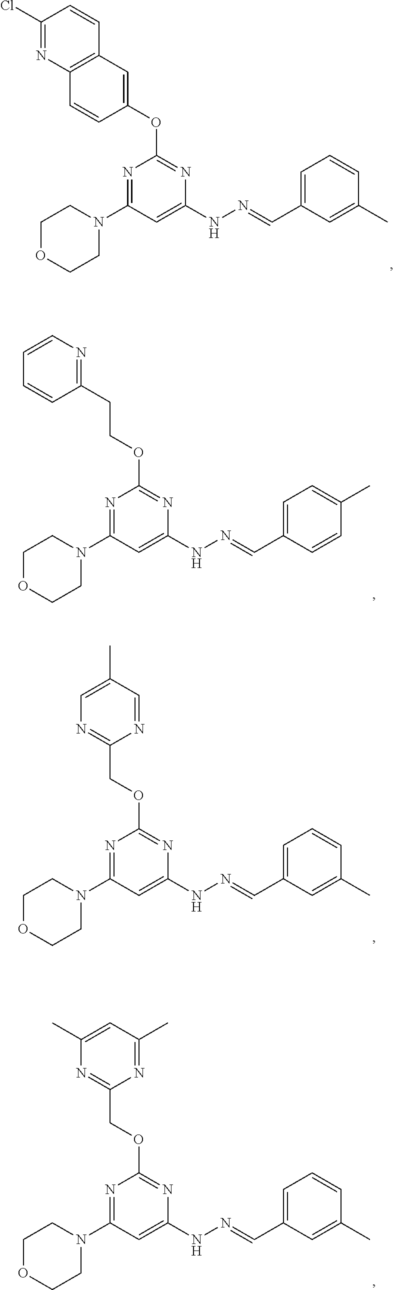 [SMILES: Cc1cccc(/C=N/Nc2cc(N3CCOCC3)nc(Oc3ccc4nc(Cl)ccc4c3)n2)c1 ; Cc1ccc(/C=N/Nc2cc(N3CCOCC3)nc(OCCc3ccccn3)n2)cc1 ; Cc1cnc(COc2nc(N/N=C/c3cccc(C)c3)cc(N3CCOCC3)n2)nc1 ; Cc1cccc(/C=N/Nc2cc(N3CCOCC3)nc(OCc3nc(C)cc(C)n3)n2)c1]

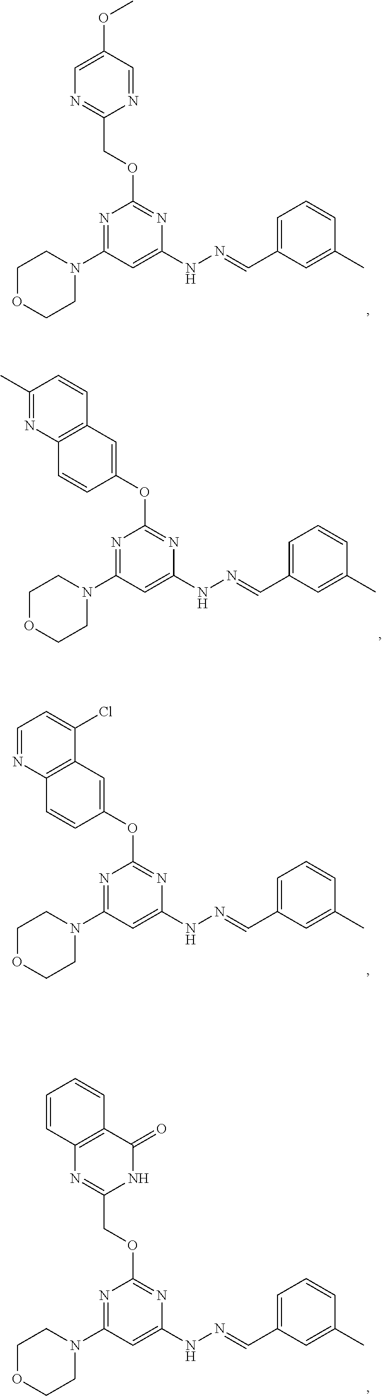

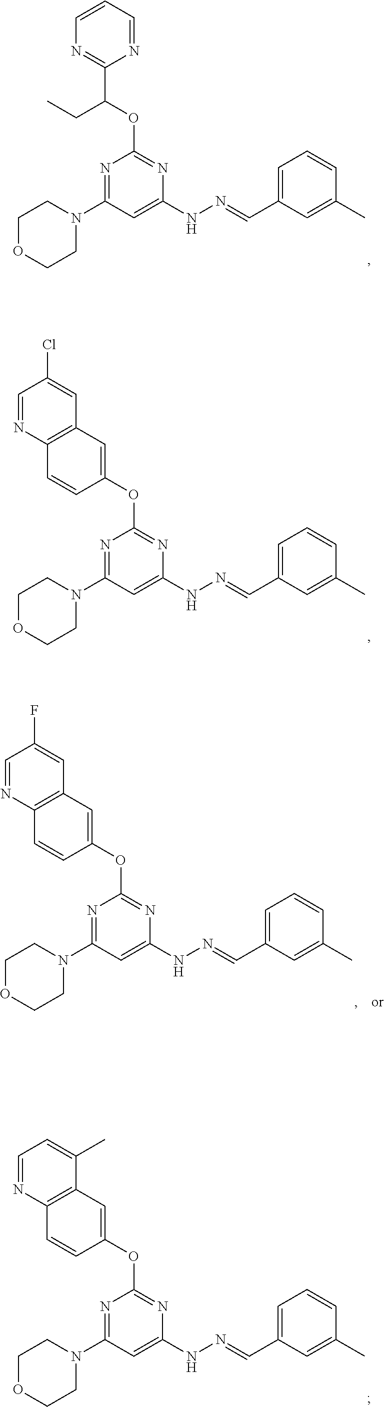 [SMILES: CCC(Oc1nc(N/N=C/c2cccc(C)c2)cc(N2CCOCC2)n1)c1ncccn1 ; Cc1cccc(/C=N/Nc2cc(N3CCOCC3)nc(Oc3ccc4ncc(Cl)cc4c3)n2)c1 ; Cc1cccc(/C=N/Nc2cc(N3CCOCC3)nc(Oc3ccc4ncc(F)cc4c3)n2)c1 ; Cc1cccc(/C=N/Nc2cc(N3CCOCC3)nc(Oc3ccc4nccc(C)c4c3)n2)c1]

or a pharmaceutically acceptable salt, solvate, or prodrug thereof.

In some embodiments is a compound having the structure:

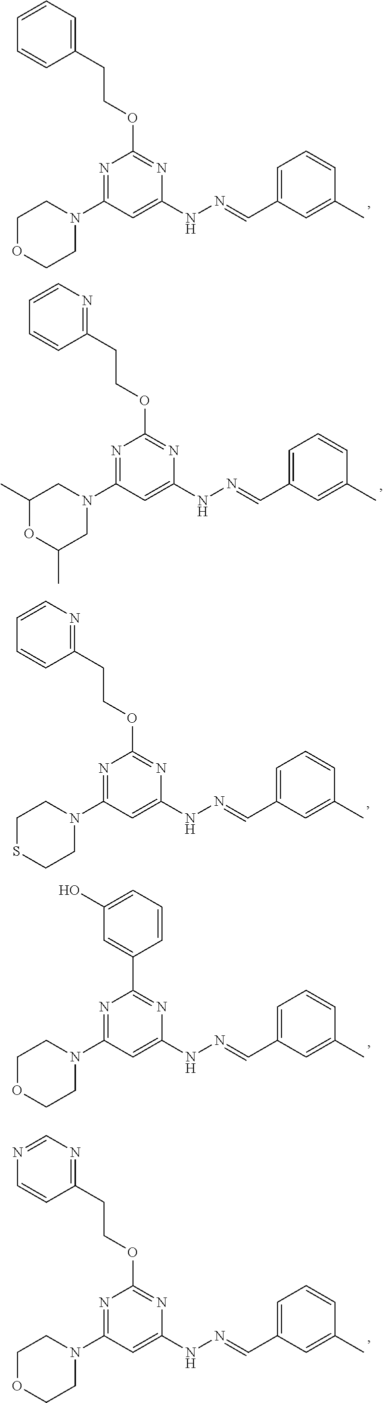

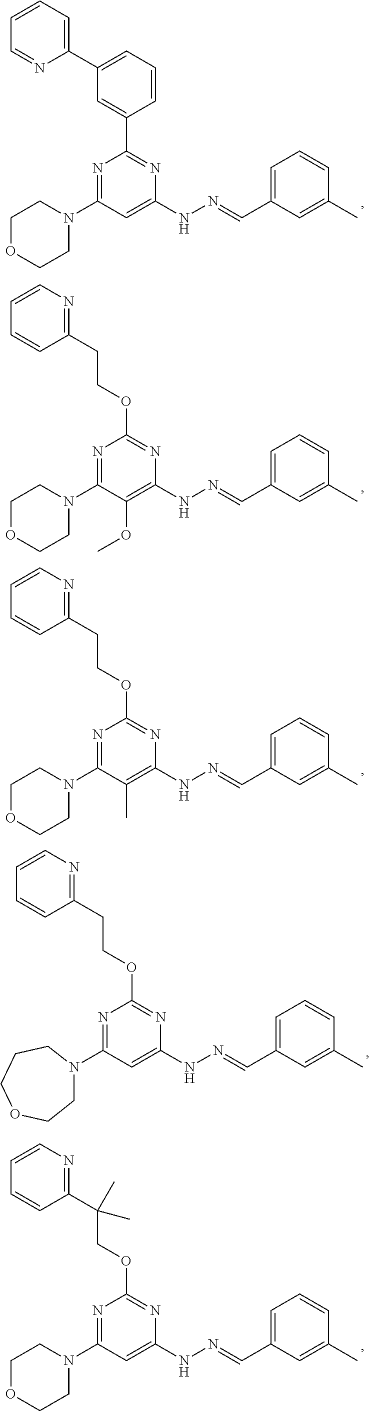

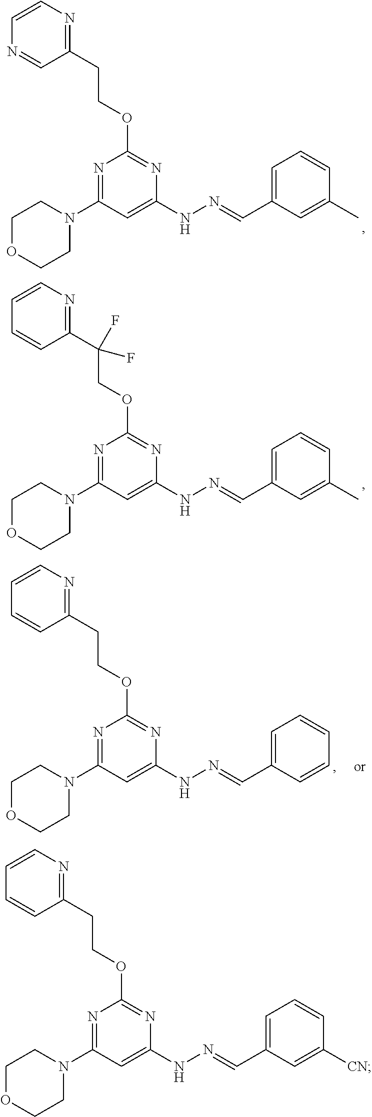

or a pharmaceutically acceptable salt, solvate, or prodrug thereof.

In some embodiments is a compound having the structure:

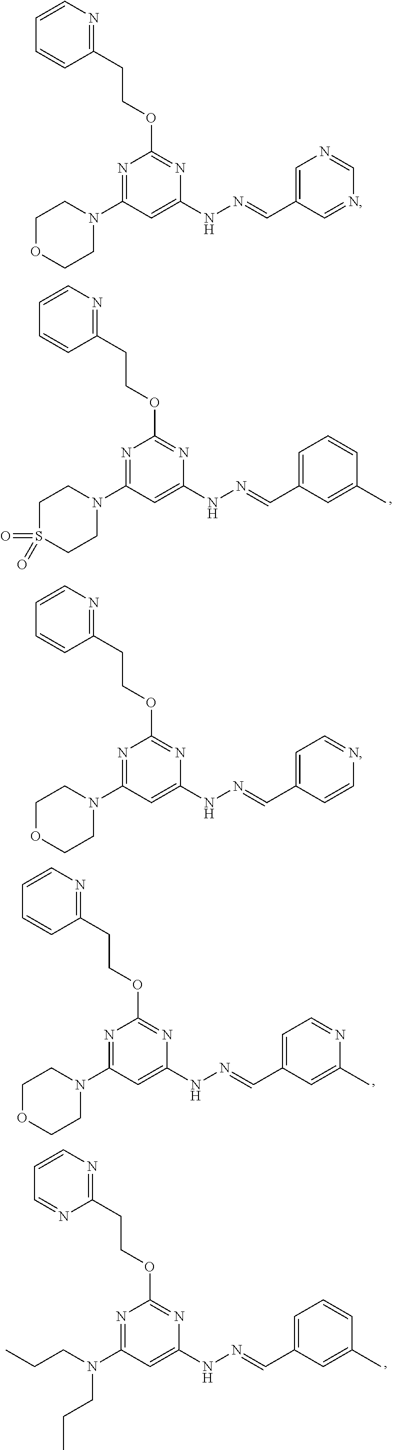

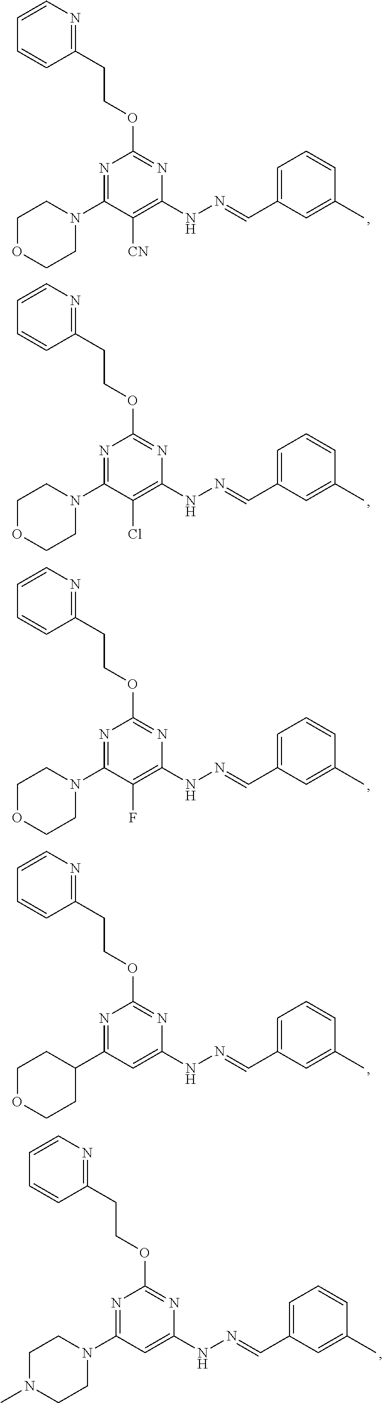

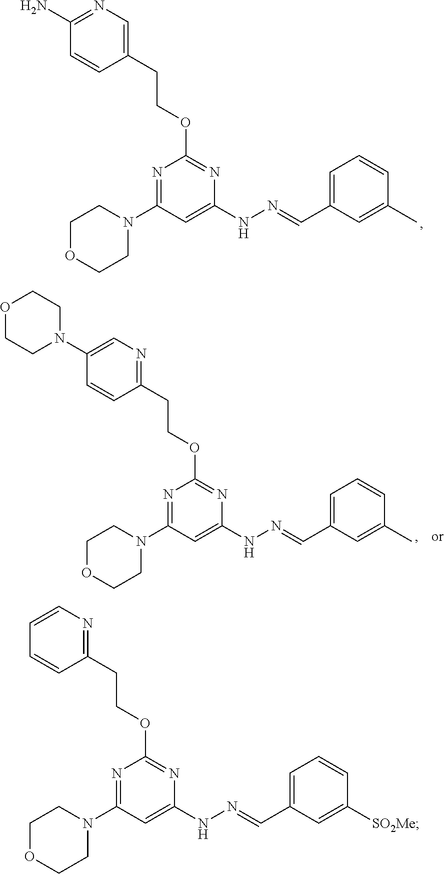

or a pharmaceutically acceptable salt, solvate, or prodrug thereof.

In some embodiments is a compound having the structure:

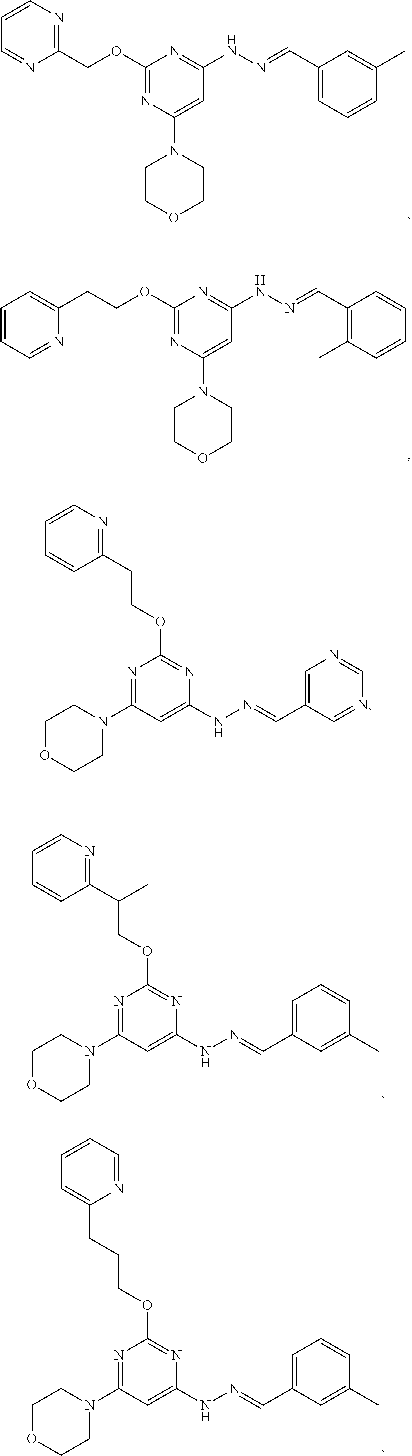 [SMILES: Cc1cccc(/C=N/Nc2cc(N3CCOCC3)nc(OCc3ncccn3)n2)c1 ; Cc1ccccc1/C=N/Nc1cc(N2CCOCC2)nc(OCCc2ccccn2)n1 ; C(=N\Nc1cc(N2CCOCC2)nc(OCCc2ccccn2)n1)/c1cncnc1 ; Cc1cccc(/C=N/Nc2cc(N3CCOCC3)nc(OCC(C)c3ccccn3)n2)c1 ; Cc1cccc(/C=N/Nc2cc(N3CCOCC3)nc(OCCCc3ccccn3)n2)c1]

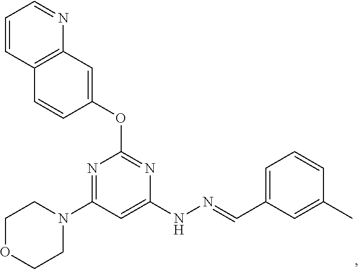 [SMILES: Cc1cccc(/C=N/Nc2cc(N3CCOCC3)nc(Oc3ccc4cccnc4c3)n2)c1]

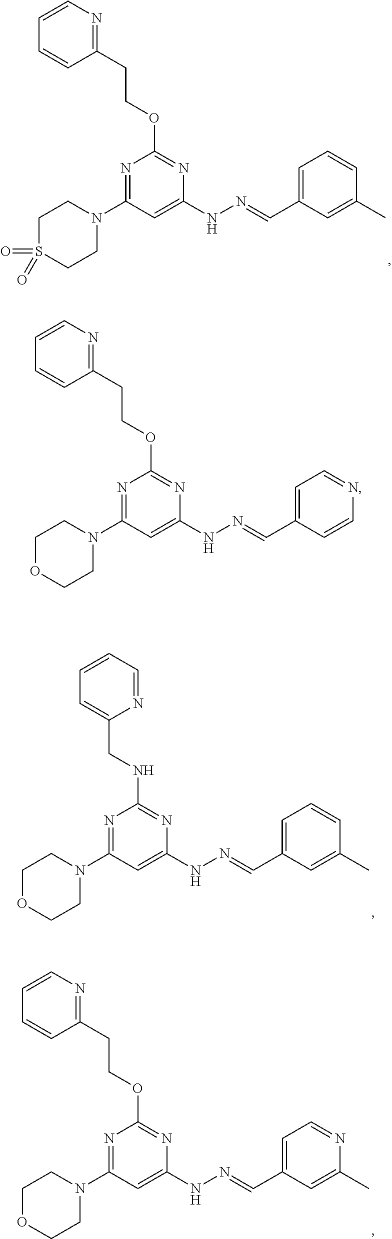 [SMILES: Cc1cccc(/C=N/Nc2cc(N3CCS(=O)(=O)CC3)nc(OCCc3ccccn3)n2)c1 ; C(=N\Nc1cc(N2CCOCC2)nc(OCCc2ccccn2)n1)/c1ccncc1 ; Cc1cccc(/C=N/Nc2cc(N3CCOCC3)nc(NCc3ccccn3)n2)c1 ; Cc1cc(/C=N/Nc2cc(N3CCOCC3)nc(OCCc3ccccn3)n2)ccn1]

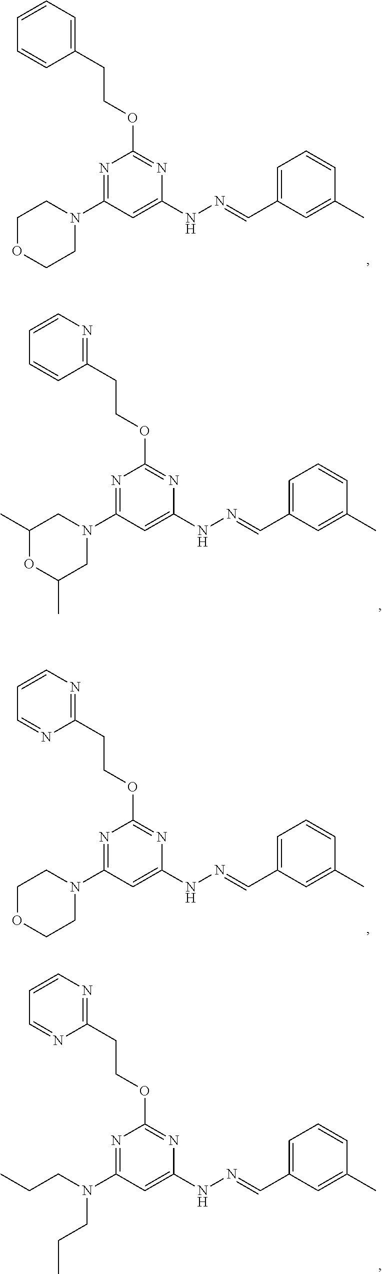

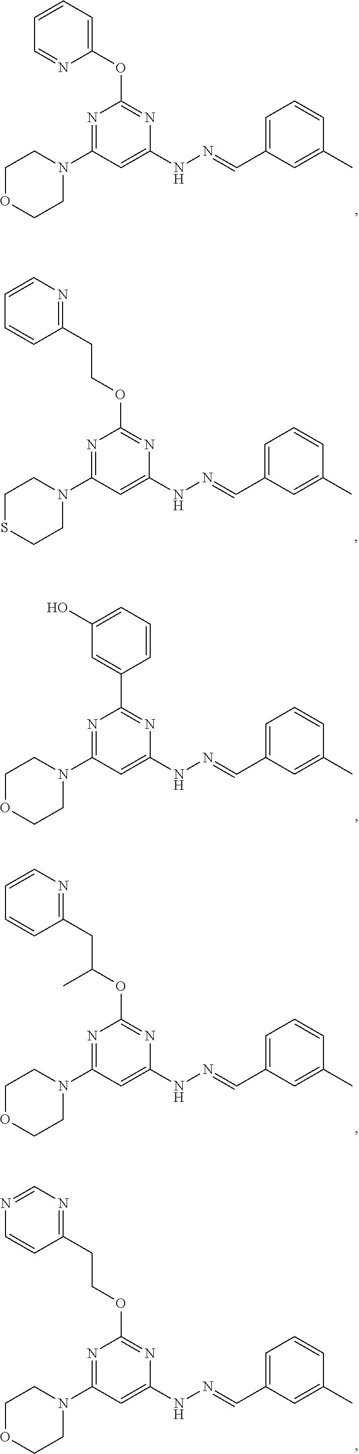

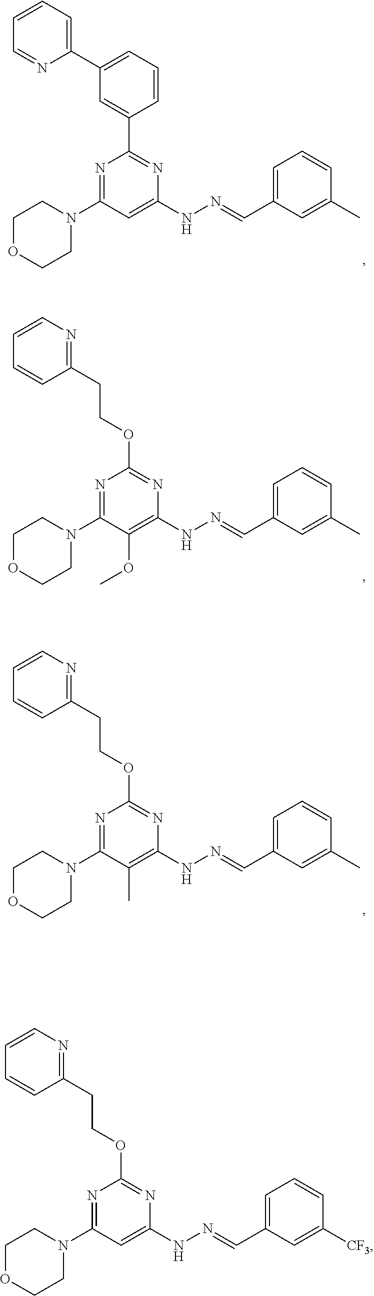 [SMILES: Cc1cccc(/C=N/Nc2cc(N3CCOCC3)nc(-c3cccc(-c4ccccn4)c3)n2)c1 ; COc1c(N/N=C/c2cccc(C)c2)nc(OCCc2ccccn2)nc1N1CCOCC1 ; Cc1cccc(/C=N/Nc2nc(OCCc3ccccn3)nc(N3CCOCC3)c2C)c1 ; FC(F)(F)c1cccc(/C=N/Nc2cc(N3CCOCC3)nc(OCCc3ccccn3)n2)c1]

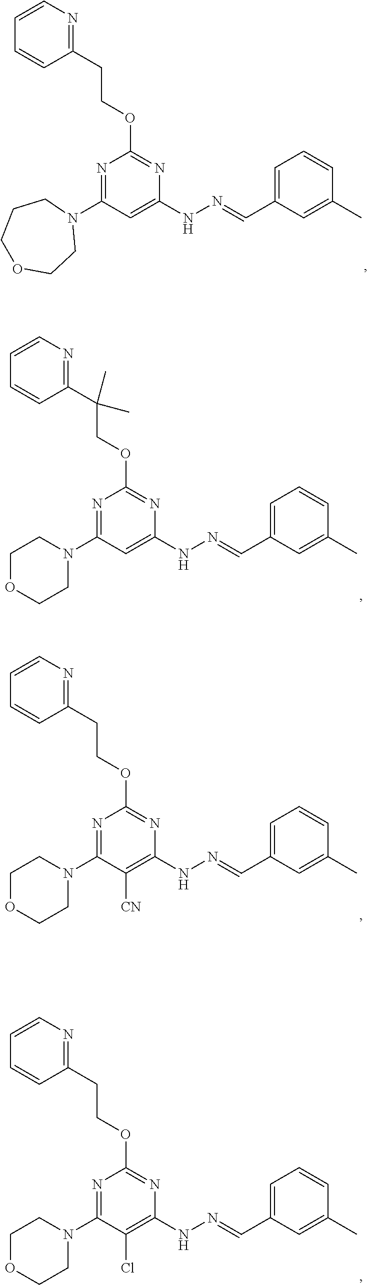 [SMILES: Cc1cccc(/C=N/Nc2cc(N3CCCOCC3)nc(OCCc3ccccn3)n2)c1 ; Cc1cccc(/C=N/Nc2cc(N3CCOCC3)nc(OCC(C)(C)c3ccccn3)n2)c1 ; Cc1cccc(/C=N/Nc2nc(OCCc3ccccn3)nc(N3CCOCC3)c2C#N)c1 ; Cc1cccc(/C=N/Nc2nc(OCCc3ccccn3)nc(N3CCOCC3)c2Cl)c1]

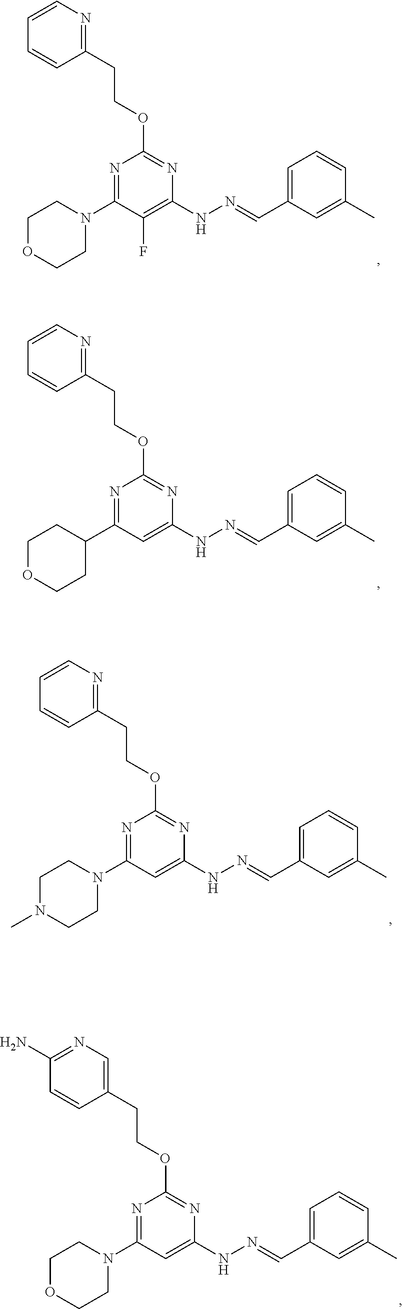

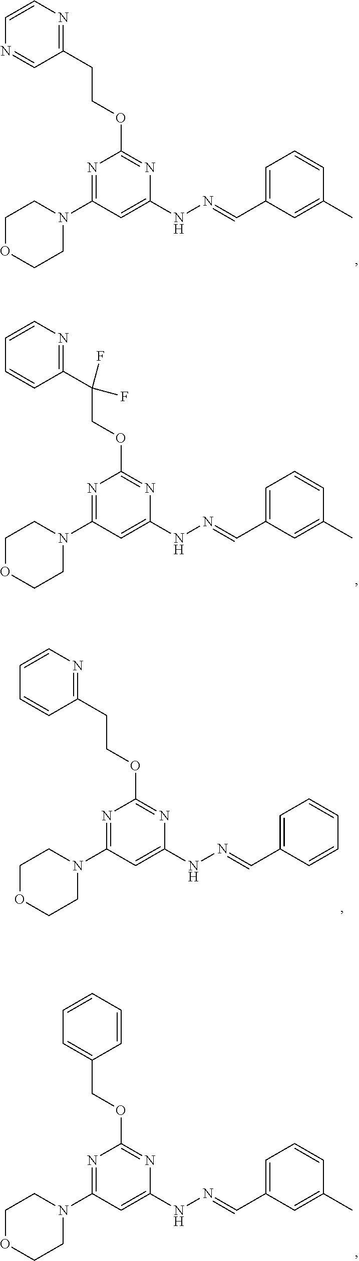 [SMILES: Cc1cccc(/C=N/Nc2cc(N3CCOCC3)nc(OCCc3cnccn3)n2)c1 ; Cc1cccc(/C=N/Nc2cc(N3CCOCC3)nc(OCC(F)(F)c3ccccn3)n2)c1 ; C(=N\Nc1cc(N2CCOCC2)nc(OCCc2ccccn2)n1)/c1ccccc1 ; Cc1cccc(/C=N/Nc2cc(N3CCOCC3)nc(OCc3ccccc3)n2)c1]

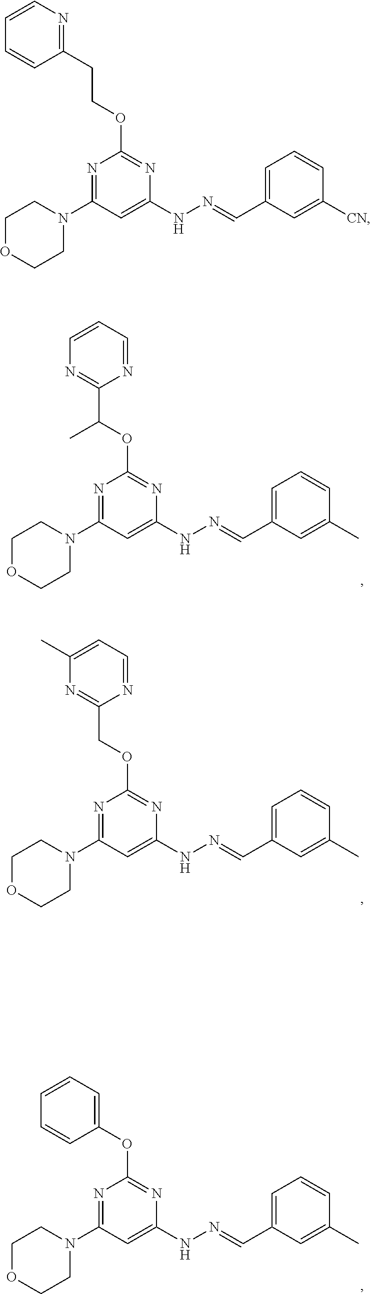 [SMILES: N#Cc1cccc(/C=N/Nc2cc(N3CCOCC3)nc(OCCc3ccccn3)n2)c1 ; Cc1cccc(/C=N/Nc2cc(N3CCOCC3)nc(OC(C)c3ncccn3)n2)c1 ; Cc1cccc(/C=N/Nc2cc(N3CCOCC3)nc(OCc3nccc(C)n3)n2)c1 ; Cc1cccc(/C=N/Nc2cc(N3CCOCC3)nc(Oc3ccccc3)n2)c1]

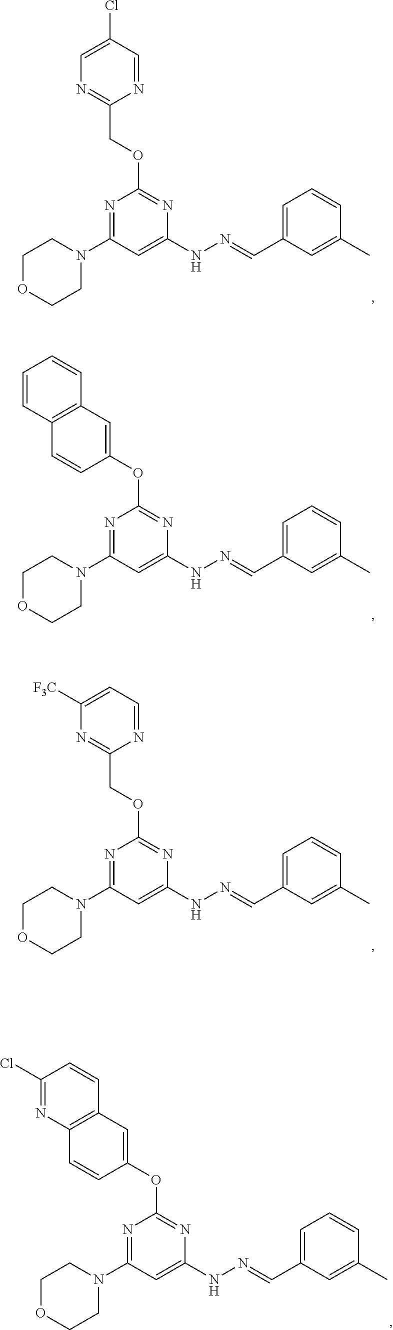 [SMILES: Cc1cccc(/C=N/Nc2cc(N3CCOCC3)nc(OCc3ncc(Cl)cn3)n2)c1 ; Cc1cccc(/C=N/Nc2cc(N3CCOCC3)nc(Oc3ccc4ccccc4c3)n2)c1 ; Cc1cccc(/C=N/Nc2cc(N3CCOCC3)nc(OCc3nccc(C(F)(F)F)n3)n2)c1 ; Cc1cccc(/C=N/Nc2cc(N3CCOCC3)nc(Oc3ccc4nc(Cl)ccc4c3)n2)c1]

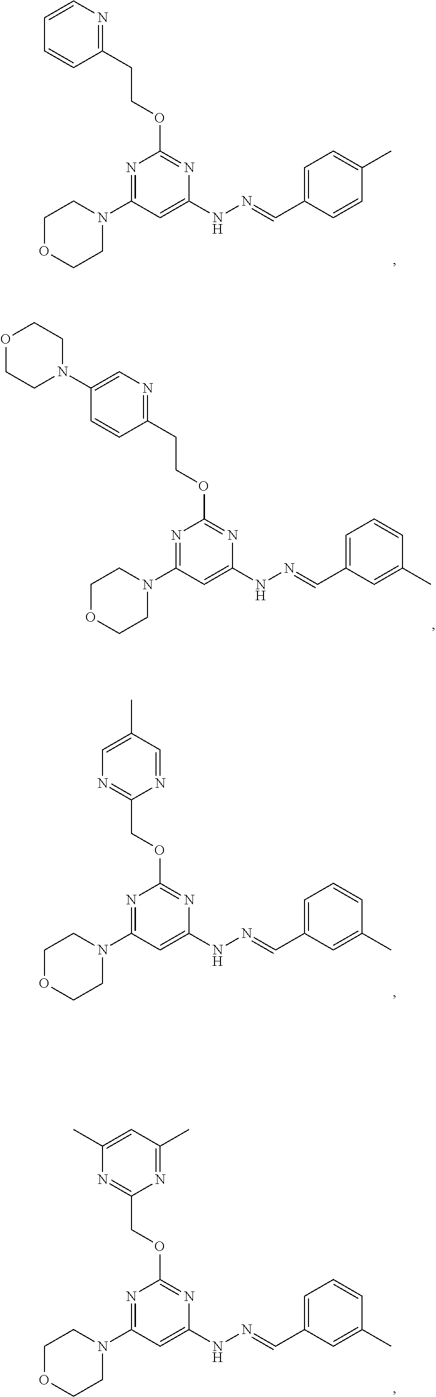 [SMILES: Cc1ccc(/C=N/Nc2cc(N3CCOCC3)nc(OCCc3ccccn3)n2)cc1 ; Cc1cccc(/C=N/Nc2cc(N3CCOCC3)nc(OCCc3ccc(N4CCOCC4)cn3)n2)c1 ; Cc1cnc(COc2nc(N/N=C/c3cccc(C)c3)cc(N3CCOCC3)n2)nc1 ; Cc1cccc(/C=N/Nc2cc(N3CCOCC3)nc(OCc3nc(C)cc(C)n3)n2)c1]

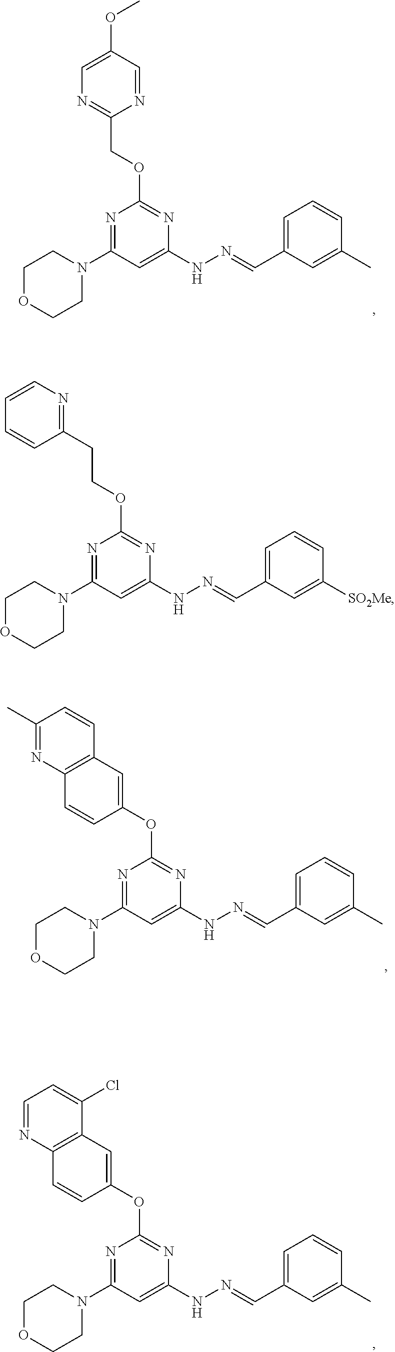

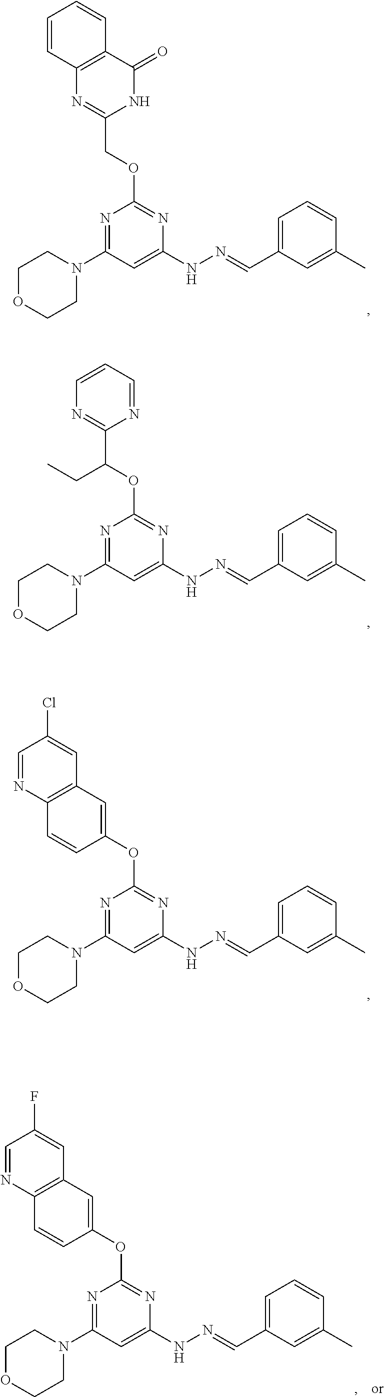 [SMILES: Cc1cccc(/C=N/Nc2cc(N3CCOCC3)nc(OCc3nc4ccccc4c(=O)[nH]3)n2)c1 ; CCC(Oc1nc(N/N=C/c2cccc(C)c2)cc(N2CCOCC2)n1)c1ncccn1 ; Cc1cccc(/C=N/Nc2cc(N3CCOCC3)nc(Oc3ccc4ncc(Cl)cc4c3)n2)c1 ; Cc1cccc(/C=N/Nc2cc(N3CCOCC3)nc(Oc3ccc4ncc(F)cc4c3)n2)c1]

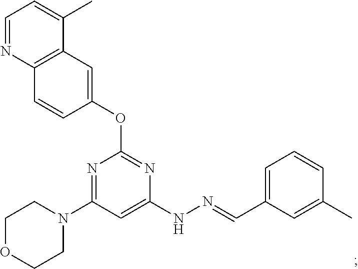 [SMILES: Cc1cccc(/C=N/Nc2cc(N3CCOCC3)nc(Oc3ccc4nccc(C)c4c3)n2)c1]

or a pharmaceutically acceptable salt, solvate, or prodrug thereof.

In another embodiment is a pharmaceutical composition comprising a compound described herein, or a pharmaceutically acceptable salt, solvate, or prodrug thereof, and a pharmaceutically acceptable excipient.

### Routes of Administration

Suitable routes of administration include, but are not limited to, oral, intravenous, aerosol, parenteral, ophthalmic, pulmonary, transmucosal, transdermal, nasal, and topical administration. In addition, by way of example only, parenteral delivery includes intramuscular, subcutaneous, intravenous, intramedullary injections, as well as intrathecal, direct intraventricular, intraperitoneal, intralymphatic, and/or intranasal injections.

In certain embodiments, a compound of Formula (I) is administered in a local rather than systemic manner, for example, via topical application of the compound directly on to skin, or intravenously, or subcutaneously, often in a depot preparation or sustained release formulation.

In specific embodiments, long acting formulations are administered by implantation (for example subcutaneously or intramuscularly) or by intramuscular injection. In yet other embodiments, the compound as described herein is provided in the form of a rapid release formulation, in the form of an extended release formulation, or in the form of an intermediate release formulation. In yet other embodiments, the compound described herein is administered topically (e.g., as a patch, an ointment, or in combination with a wound dressing, or as a wash or a spray). In alternative embodiments, a formulation is administered systemically (e.g., by injection, or as a pill).

### Pharmaceutical Compositions/Formulations

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a compound of Formula (I) or (II) and at least one pharmaceutically acceptable inactive ingredient. In some embodiments, the compounds described herein are administered as pharmaceutical compositions in which compounds of Formula (I) or (II) are mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound of Formula (I) or (II) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound of Formula (I) or (II) are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formula (I) or (II) as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds of Formula (I) or (II) exist in unsolvated form or in solvated forms with pharmaceutically acceptable solvents such as water, ethanol, and the like. The solvated forms of the compounds of Formula (I) or (II) are also considered to be disclosed herein.

In some embodiments, the compounds of Formula (I) or (II) exist as tautomers. All tautomers are included within the scope of the compounds presented herein. As such, it is to be understood that a compound of the Formula (I) or (II) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein.

In some embodiments, compounds of Formula (I) or (II) exist as enantiomers, diastereomers, or other steroisomeric forms. The compounds disclosed herein include all enantiomeric, diastereomeric, and epimeric forms as well as mixtures thereof.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrug forms of the herein described compounds, wherein the prodrug is metabolized in vivo to produce a compound of (I) as set forth herein are included within the scope of the claims. Prodrug forms of the herein described compounds, wherein the prodrug is metabolized in vivo to produce a compound of Formula (I) or (II) as set forth herein are included within the scope of the claims. In some cases, some of the compounds described herein may be a prodrug for another derivative or active compound. In some embodiments described herein, hydrazones are metabolized in vivo to produce a compound of Formula (I) or (II).

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein, which include a compound of Formula (I) or (II) are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Certain Topical Compositions

In some embodiments, compounds of Formula (I) or (II) are prepared as transdermal dosage forms. In one embodiment, the transdermal formulations described herein include at least three components: (1) a formulation of a compound of Formula (I) or (II); (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the compounds described herein can be accomplished by means of iontophoretic patches and the like. In one aspect, transdermal patches provide controlled delivery of a compound of Formula (I) or (II). In one aspect, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In alternative embodiments, a compound of Formula (I) or (II) is formulated and presented as a wash or rinse liquid which is used to irrigate the affected area. In further embodiments, a compound of Formula (I) or (II) is formulated and presented as a spray which is applied to the affected area.

### Certain Systemically Administered Compositions

In one aspect, a compound of Formula (I) or (II) is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions.

Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation.

Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a compound of Formula (I) or (II) is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formula (I) or (II) are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

In some embodiments, pharmaceutical formulations of a compound of Formula (I) or (II) are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added. A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration.

In one aspect, solid oral dosage forms are prepared by mixing a compound of Formula (I) or (II) with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder.

Compressed tablets are solid dosage forms prepared by compacting the bulk blend of the formulations described above. In various embodiments, tablets will include one or more flavoring agents.

In other embodiments, the tablets will include a film surrounding the final compressed tablet. In some embodiments, the film coating can provide a delayed release of the compound of Formula (I) or (II) from the formulation. In other embodiments, the film coating aids in patient compliance (e.g., Opadry® coatings or sugar coating). Film coatings including Opadry® typically range from about 1% to about 3% of the tablet weight.

In some embodiments, solid dosage forms, e.g., tablets, effervescent tablets, and capsules, are prepared by mixing particles of a compound with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD 100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to an antiviral compound the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a compound of Formula (I) or (II) are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, an antiviral compound is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion.

Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethyl cellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethyl cellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a compound of Formula (I) or (II) and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a compound of Formula (I) or (II) is formulated to include one or more pharmaceutical excipients and flavors. Such a powder is prepared, for example, by mixing the compound and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

### Controlled Release Formulations

In some embodiments, the pharmaceutical dosage forms are formulated to provide a controlled release of a compound of Formula (I) or (II). Controlled release refers to the release of the compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a compound of Formula (I) or (II), that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following:

Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids.

Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of the compound of Formula (I) or (II) upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a compound of Formula (I) or (II) and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a compound of Formula (I) or (II), the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween®60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), maltol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

### Methods of Dosing and Treatment Regimens

A method for treating any of the diseases or conditions described herein in a subject in need of such treatment, involves administration of pharmaceutical compositions that include at least one compound of Formula (I) or (II) or a pharmaceutically acceptable salt, pharmaceutically acceptable prodrug, or pharmaceutically acceptable solvate thereof, in therapeutically effective amounts to said subject. In another embodiment, the compounds of Formula (I) or (II) are used in the preparation of medicaments for treating a viral infection. In another embodiment, the compounds of Formula (I) or (II) are used in the preparation of medicaments for treating an RNA virus infection. In some embodiments, the compounds of Formula (I) or (II) are used in the preparation of medicaments for treating Ebola virus infection.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like.

When used in a patient, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound of Formula (I) or (II) in order to prevent a return of the symptoms of the disease or condition.

In certain embodiments wherein the patient's condition does not improve, upon the doctor's discretion the administration of the compound of Formula (I) or (II) is administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of an antiviral compound, the patient is also weaned off (e.g., step-wise decrease in dose) a second treatment regimen.

In one embodiment, the daily dosages appropriate for a compound of Formula (I) or (II) described herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In one embodiment, the daily dosage is administered in extended release form. In certain embodiments, suitable unit dosage forms for oral administration comprise from about 1 to 500 mg active ingredient. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Combination Treatment

The compounds according to Formula (I) or (II) described herein may be used in combination with one or more additional agents. In some embodiments is a method for treating a viral infection comprising administering to an individual in need thereof a therapeutically effective amount of a compound of Formula (I) or (II) and one or more additional agents selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, and T-705. In some embodiments is a method for treating a viral infection comprising administering to an individual in need thereof a therapeutically effective amount of a compound of Formula (I) or (II) and one or more additional agents selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, and T-705, and GS-5734. In some embodiments is a method for treating a viral infection comprising administering to an individual in need thereof a therapeutically effective amount of a compound of Formula (I) or (II) and Ribavirin. In some embodiments is a method for treating a viral infection comprising administering to an individual in need thereof a therapeutically effective amount of a compound of Formula (I) or (II) and Fenretinide. In some embodiments is a method for treating a viral infection comprising administering to an individual in need thereof a therapeutically effective amount of a compound of Formula (I) or (II) and Favipiravir. In some embodiments is a method for treating a viral infection comprising administering to an individual in need thereof a therapeutically effective amount of a compound of Formula (I) or (II) and Brincidofovir. In some embodiments is a method for treating a viral infection comprising administering to an individual in need thereof a therapeutically effective amount of a compound of Formula (I) or (II) and ZMapp. In some embodiments is a method for treating a viral infection comprising administering to an individual in need thereof a therapeutically effective amount of a compound of Formula (I) or (II) and TKM-100802. In some embodiments is a method for treating a viral infection comprising administering to an individual in need thereof a therapeutically effective amount of a compound of Formula (I) or (II) and BCX4430. In some embodiments is a method for treating a viral infection comprising administering to an individual in need thereof a therapeutically effective amount of a compound of Formula (I) or (II) and Interferons. In some embodiments is a method for treating a viral infection comprising administering to an individual in need thereof a therapeutically effective amount of a compound of Formula (I) or (II) and Amiodarone. In some embodiments is a method for treating a viral infection comprising administering to an individual in need thereof a therapeutically effective amount of a compound of Formula (I) or (II) and Atorvostatin. In some embodiments is a method for treating a viral infection comprising administering to an individual in need thereof a therapeutically effective amount of a compound of Formula (I) or (II) and Irbesartan. In some embodiments is a method for treating a viral infection comprising administering to an individual in need thereof a therapeutically effective amount of a compound of Formula (I) or (II) and Clomiphene. In some embodiments is a method for treating a viral infection comprising administering to an individual in need thereof a therapeutically effective amount of a compound of Formula (I) or (II) and FX06. In some embodiments is a method for treating a viral infection comprising administering to an individual in need thereof a therapeutically effective amount of a compound of Formula (I) or (II) and Zmab. In some embodiments is a method for treating a viral infection comprising administering to an individual in need thereof a therapeutically effective amount of a compound of Formula (I) or (II) and Tamoxifen. In some embodiments is a method for treating a viral infection comprising administering to an individual in need thereof a therapeutically effective amount of a compound of Formula (I) or (II) and Albendazole. In some embodiments is a method for treating a viral infection comprising administering to an individual in need thereof a therapeutically effective amount of a compound of Formula (I) or (II) and AC-93253. In some embodiments is a method for treating a viral infection comprising administering to an individual in need thereof a therapeutically effective amount of a compound of Formula (I) or (II) and Toremifene. In some embodiments is a method for treating a viral infection comprising administering to an individual in need thereof a therapeutically effective amount of a compound of Formula (I) or (II) and T-705. In some embodiments is a method for treating a viral infection comprising administering to an individual in need thereof a therapeutically effective amount of a compound of Formula (I) or (II) and GS-5734. In any of the aforementioned embodiments for treating a viral infection, the viral infection is an RNA virus infection. In any of the aforementioned embodiments for treating a viral infection, the viral infection is an Ebola virus infection.

In some embodiments, the compound of Formula (I) or (II) and the one or more additional agents are administered concurrently (simultaneously, essentially simultaneously, or withing the same treatment protocol) or sequentially, depending upon the nature of the diseases, the condition of the patient, and the actual choice of compounds used.

In certain embodiments, the determination of the order of administration, and the number of repetitions of administration of each therapeutic agent during a treatment protocol, is based upon evaluation of the disease being treated and the condition of the patient.

In certain embodiments, the compound of Formula (I) or (II) and the one or more additional agents are part of the same composition (fixed combination). In some embodiments, the compound of Formula (I) or (II) and the one or more additional agents are administered as different compositions (non-fixed combinations). In another embodiment, the compound of Formula (I) or (II) is administered prior to the one or more additional agents. In some embodiments, the one or more additional agents are administered prior to the compound of Formula (I) or (II).

### EXAMPLES

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants (J) are reported in Hertz. For proton spectra the solvent peak was used as the reference peak.

### Example 1: (E)-4-(6-(2-(3-Methylbenzylidene)hydrazinyl)-2-(pyrimidin-2-ylmethoxy)pyrimidin-4-yl)morpholine (5)

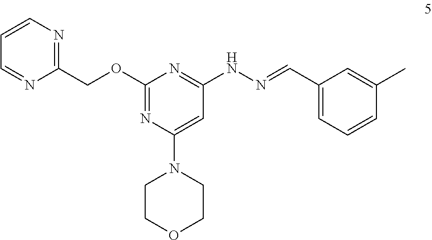 [SMILES: Cc1cccc(/C=N/Nc2cc(N3CCOCC3)nc(OCc3ncccn3)n2)c1]

### Step 1: Preparation of 4,6-dichloro-2-(pyrimidin-2-ylmethoxy)pyrimidine (2)

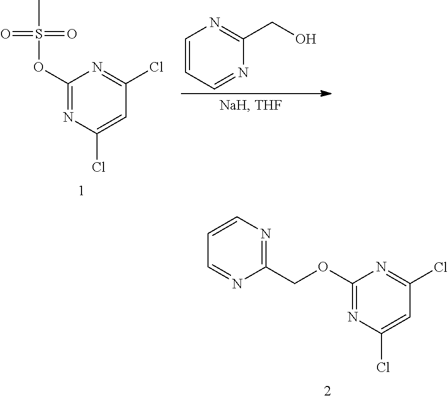

To a solution of compound 1 (2.0 g, 8.23 mmol) and pyrimidin-2-ylmethanol (924 mg, 8.39 mmol) in THF (10 mL) at −78° C. was added NaH (494 mg, 12.35 mmol) portionwise. The reaction mixture was warmed to 25° C. and stirred for 16 h. The reaction was quenched with H₂O (8 mL) and the mixture was extracted with ethyl acetate (10 mL×3). The combined organic extracts were concentrated to give a residue. The residue was purified by silica gel (10-50% EtOAc in hexanes) to give 4,6-dichloro-2-(pyrimidin-2-ylmethoxy)pyrimidine (2) (1.26 g, 60%) as a white solid. ¹H NMR (400 MHz, CDCl₃) δ 8.74 (d, J=4.8 Hz, 2H), 7.23-7.25 (m, 1H), 7.06 (s, 1H), 5.69 (s, 2H).

### Step 2: Preparation of 4-(6-chloro-2-(pyrimidin-2-ylmethoxy)pyrimidin-4-yl)morpholine (3)

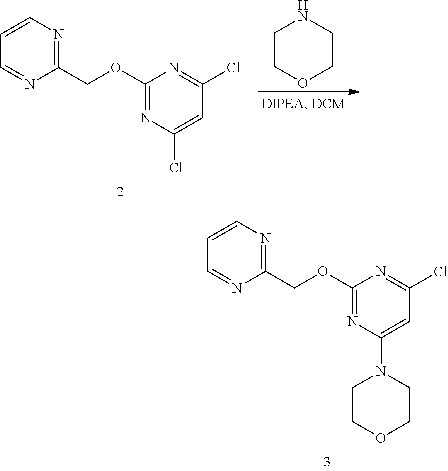

To a solution of compound 2 (1.26 g, 4.90 mmol) and morpholine (427 mg, 4.90 mmol) in CH₂Cl₂ (10 mL) was added DIPEA (1.03 mL, 5.88 mmol). The reaction mixture was stirred at ambient temperature for 20 h. Solvent was removed under reduced pressure to give a residue which was purified by column chromatography (10-50% EtOAc in hexanes) to give 4-(6-chloro-2-(pyrimidin-2-ylmethoxy)pyrimidin-4-yl)morpholine (3) (935 mg, 62%) as a white solid. ¹H NMR (400 MHz, CD₃OD) δ 8.75 (d, J=5.2 Hz, 2H), 7.39 (t, J=4.8 Hz, 1H), 6.44 (s, 1H), 5.49 (s, 2H), 3.61-3.63 (m, 4H), 3.49-3.51 (m, 4H).

### Step 3: Preparation of 4-(6-hydrazinyl-2-(pyrimidin-2-ylmethoxy)pyrimidin-4-yl)morpholine (4)

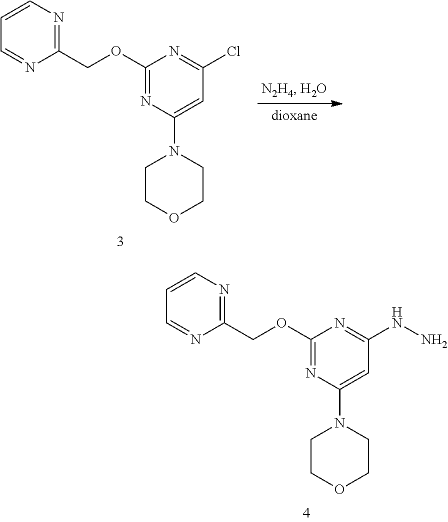 [SMILES: Clc1cc(N2CCOCC2)nc(OCc2ncccn2)n1 ; NNc1cc(N2CCOCC2)nc(OCc2ncccn2)n1]

To a solution of compound 3 (935 mg, 3.04 mmol) in dioxane (10 mL) was added N₂H₄.H₂O (993 mg, 19.84 mmol). The reaction mixture was stirred at 80° C. heating block for 16 h. Solvent was removed under reduced pressure to give a residue. The residue was dissolved in EtOAc (50 mL) and washed with water (10 mL). Solvent was removed under reduced pressure to give 4-(6-hydrazinyl-2-(pyrimidin-2-ylmethoxy)pyrimidin-4-yl)morpholine (4) (640 mg, 69%) as a white solid which was used without further purification. ¹H NMR (400 MHz, CDCl₃) δ 8.72 (s, 2H), 7.18 (s, 1H), 6.19 (s, 1H), 5.50-5.52 (m, 2H), 3.68 (s, 4H), 3.45 (s, 4H), 1.97 (s, 2H).

### Step 4: Preparation of (E)-4-(6-(2-(3-methylbenzylidene)hydrazinyl)-2-(pyrimidin-2-ylmethoxy)pyrimidin-4-yl)morpholine (5)

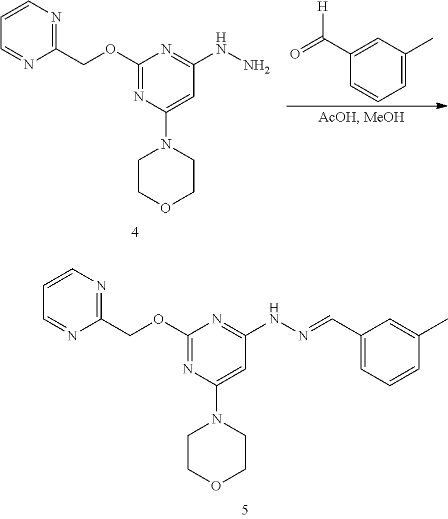

To a solution of compound 4 (300 mg, 0.989 mmol) and 3-methylbenzaldehyde (131 mg, 1.09 mmol) in MeOH (8 mL) was added AcOH (0.1 mL). The reaction was stirred at ambient temperature for 16 h. The mixture was concentrated and the residue was purified by column chromatography (10-50% EtOAc in hexanes) to give (E)-4-(6-(2-(3-methylbenzylidene)hydrazinyl)-2-(pyrimidin-2-ylmethoxy)pyrimidin-4-yl)morpholine (5) (50 mg, 12%) as a light yellow foam. ¹H NMR (400 MHz, CD₃OD) δ 8.78 (dd, J=4.9, 1.6 Hz, 2H), 7.91 (d, J=1.6 Hz, 1H), 7.55-7.45 (m, 2H), 7.41 (td, J=5.0, 1.6 Hz, 1H), 7.28 (td, J=7.6, 1.5 Hz, 1H), 7.19 (d, J=7.8 Hz, 1H), 6.11 (d, J=1.6 Hz, 1H), 5.49 (d, J=1.6 Hz, 2H), 3.74-3.62 (m, 4H), 3.55-3.44 (m, 4H), 2.39 (d, J=1.8 Hz, 3H). MS-ESI: m/z 406.18 observed (M+H)⁺.

### Example 2: (E)-4-(6-(2-(2-Methylbenzylidene)hydrazinyl)-2-(2-(pyridin-2-yl)ethoxy)pyrimidin-4-yl)morpholine (10)

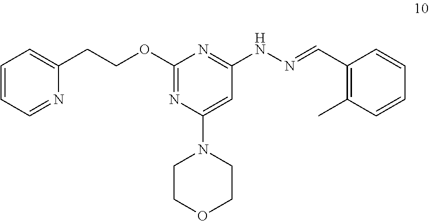 [SMILES: Cc1ccccc1/C=N/Nc1cc(N2CCOCC2)nc(OCCc2ccccn2)n1]

Step 1: Preparation of 4-(2,6-dichloropyrimidin-4-yl)morpholine (7)

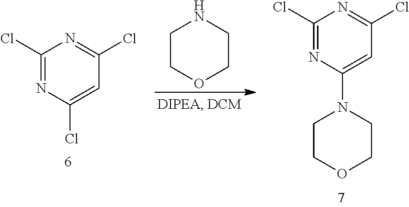

To a solution of morpholine (9.56 g, 109 mmol) in ethanol (400 mL) at 0° C. was added diisopropylethylamine (22.7 mL, 130 mmol). To this mixture was added 2,4,6-trichloropyrimidine 6 (20 g, 109 mmol) and the mixture was stirred at −20° C. for 30 minutes. The reaction mixture was concentrated. The residue was dissolved in CH₂Cl₂ and washed with water. The organic phases were dried over MgSO₄ and concentrated under reduced pressure. The residue was purified by column chromatography (5-15% EtOAc in hexanes) to give 4-(2,6-dichloropyrimidin-4-yl)morpholine (7) (25.6 g, 78%) as a white solid. MS-ESI: m/z 234 (M+H)⁺.

### Step 2: Preparation of 4-(6-chloro-2-(2-(pyridin-2-yl)ethoxy)pyrimidin-4-yl)morpholine (8)

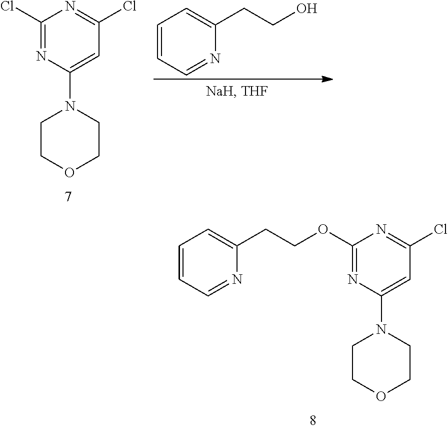

To a mixture of sodium hydride 60% in mineral oil (566 mg, 11.8 mmol) in anhydrous DMF (50 mL) at 0° C. was added 2-(pyridin-2-yl)ethan-1-ol (1.3 g, 10.72 mmol). The mixture was stirred for 15 min at 0° C. The reaction mixture was cooled to −20° C. and compound 7 (2.5 g, 10.72 mmol) was added portion-wise. The reaction mixture was stirred at 0° C. for 45 min. The reaction mixture was quenched with ice-water and extracted with EtOAc. The organic layer was separated and washed with saturated brine solution, dried over Na₂SO₄ and concentrated. The crude product was purified by column chromatography (0-5% MeOH in DCM) to give 4-(6-chloro-2-(2-(pyridin-2-yl)ethoxy)pyrimidin-4-yl)morpholine (8) (2.0 g, 59%). ¹H NMR (400 MHz, DMSO-d6) δ 8.46 (d, 1H), 7.68-7.72 (m, 1H), 7.30 (d, 1H), 7.20-7.23 (m, 1H), 5.60 (s, 1H), 4.48 (t, 2H), 3.63-3.61 (m, 4H), 3.38-3.33 (m, 4H), 3.11 (t, 2H). MS-ESI: m/z 321.1 [M+1].

### Step 3: Preparation of 4-(6-hydrazinyl-2-(2-(pyridin-2-yl)ethoxy)pyrimidin-4-yl)morpholine (9)

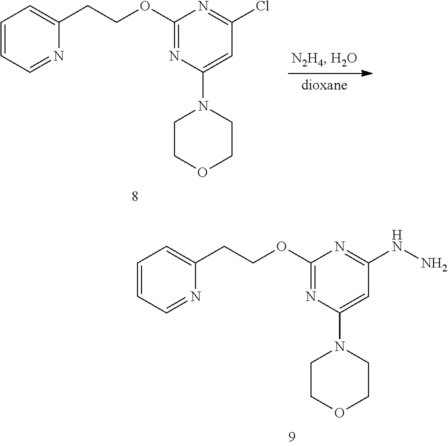 [SMILES: Clc1cc(N2CCOCC2)nc(OCCc2ccccn2)n1 ; NNc1cc(N2CCOCC2)nc(OCCc2ccccn2)n1]

To a solution of compound 8 (1.0 mg, 3.12 mmol) in dioxane (10 mL) was added N₂H₄.H₂O (0.45 mL, 9.37 mmol). The reaction mixture was stirred at 95° C. overnight. The reaction mixture was cooled to room temperature and the solvent was removed under reduced pressure. The isolated solid was washed with diethyl ether (5 mL) and dried to give 4-(6-hydrazinyl-2-(2-(pyridin-2-yl)ethoxy)pyrimidin-4-yl)morpholine (9) (700 mg, 71%) as a white solid which was used without further purification. MS-ESI: m/z 317 [M+1].

### Step 4: Preparation of (E)-4-(6-(2-(2-methylbenzylidene)hydrazinyl)-2-(2-(pyridin-2-yl)ethoxy)pyrimidin-4-yl)morpholine (10)

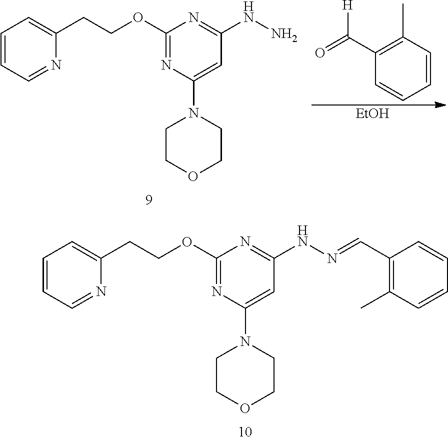

To a solution of compound 9 (100 mg, 0.31 mmol) in EtOH (5 mL) was added 2-methylbenzaldehyde (0.4 mL, 0.34 mmol). The reaction mixture was stirred at ambient temperature for 45 minutes. The mixture was concentrated and the residue was crystallized from diethyl ether to give (E)-4-(6-(2-(2-methylbenzylidene)hydrazinyl)-2-(2-(pyridin-2-yl)ethoxy)pyrimidin-4-yl)morpholine (10) (35 mg, 26%) as a white solid. ¹H NMR (400 MHz, DMSO-d₆) δ 10.84 (s, 1H), 8.52 (dt, J=4.7, 1.4 Hz, 1H), 8.33 (s, 1H), 7.89-7.80 (m, 1H), 7.73 (td, J=7.7, 1.9 Hz, 1H), 7.34 (d, J=7.8 Hz, 1H), 7.28-7.19 (m, 4H), 6.05 (s, 1H), 4.55 (t, J=6.7 Hz, 2H), 3.66 (t, J=4.7 Hz, 4H), 3.51 (t, J=4.8 Hz, 4H), 3.15 (t, J=6.8 Hz, 2H), 2.41 (s, 3H). MS-ESI: m/z 419.17 observed (M+H)⁺.

### Example 3: (E)-4-(6-(2-(3-Methylbenzylidene)hydrazinyl)-2-(2-(pyridin-2-yl)ethoxy)pyrimidin-4-yl)morpholine (11) (kBNC350)

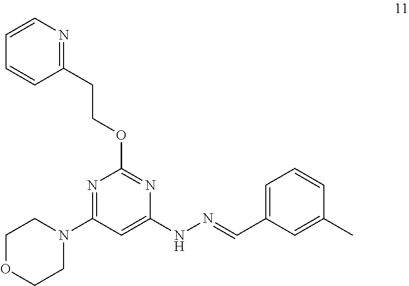 [SMILES: Cc1cccc(/C=N/Nc2cc(N3CCOCC3)nc(OCCc3ccccn3)n2)c1]

Compound 11 was prepared in a similar manner as outlined in Example 1 by using 2-(pyridin-2-yl)ethanol in step 1.

### Example 4: (E)-4-(2-(2-(Pyridin-2-yl)ethoxy)-6-(2-(pyrimidin-5-ylmethylene)hydrazinyl)pyrimidin-4-yl)morpholine (12)

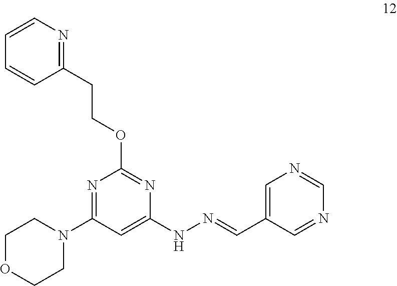 [SMILES: C(=N\Nc1cc(N2CCOCC2)nc(OCCc2ccccn2)n1)/c1cncnc1]

Compound 12 was prepared in a similar manner as outlined in Example 1 by using 2-(pyridin-2-yl)ethanol in step 1 and pyrimidine-5-carbaldehyde in step 4. MS-ESI: m/z 407.22 observed (M+H)⁺.

### Example 5: (E)-4-(6-(2-(3-Methylbenzylidene)hydrazinyl)-2-(2-(pyridin-2-yl)propoxy)pyrimidin-4-yl)morpholine (13)

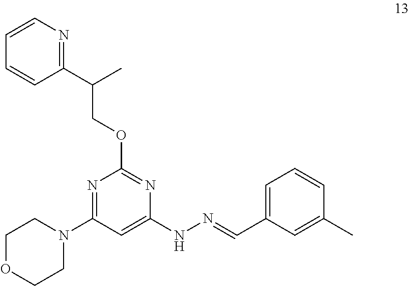 [SMILES: Cc1cccc(/C=N/Nc2cc(N3CCOCC3)nc(OCC(C)c3ccccn3)n2)c1]

Compound 13 was prepared in a similar manner as outlined in Example 1 by using 2-(pyridin-2-yl)propan-1-ol in step 1. ¹H NMR (400 MHz, CD₃OD) δ 8.49 (ddd, J=5.0, 1.9, 1.0 Hz, 1H), 7.90 (s, 1H), 7.80 (td, J=7.7, 1.8 Hz, 1H), 7.55-7.45 (m, 2H), 7.46-7.32 (m, 1H), 7.33-7.21 (m, 2H), 7.19 (d, J=7.5 Hz, 1H), 6.11 (s, 1H), 4.63-4.40 (m, 2H), 3.83-3.66 (m, 4H), 3.66-3.48 (m, 4H), 3.50-3.38 (m, 1H), 2.39 (s, 3H), 1.40 (d, J=7.0 Hz, 3H). MS-ESI: m/z 433.28 observed (M+H)⁺.

### Example 6: (E)-4-(6-(2-(3-Methylbenzylidene)hydrazinyl)-2-(3-(pyridin-2-yl)propoxy)pyrimidin-4-yl)morpholine (14)

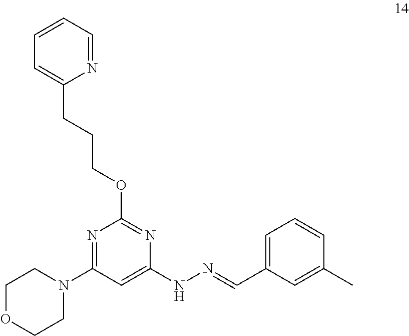 [SMILES: Cc1cccc(/C=N/Nc2cc(N3CCOCC3)nc(OCCCc3ccccn3)n2)c1]

Compound 14 was prepared in a similar manner as outlined in Example 1 by using 3-(pyridin-2-yl)propan-1-ol in step 1. ¹H NMR (400 MHz, CD₃OD) δ 8.49-8.41 (m, 1H), 7.92 (s, 1H), 7.78 (td, J=7.7, 1.9 Hz, 1H), 7.55-7.46 (m, 2H), 7.41-7.34 (m, 1H), 7.33-7.24 (m, 2H), 7.19 (d, J=7.5 Hz, 1H), 6.13 (s, 1H), 4.32 (t, J=6.4 Hz, 2H), 3.83-3.70 (m, 4H), 3.65-3.55 (m, 4H), 2.97 (t, J=7.7 Hz, 2H), 2.39 (s, 3H), 2.25-2.11 (m, 2H). MS-ESI: m z 433.28 observed (M+H)⁺.

### Example 7: (E)-4-(6-(2-(3-Methylbenzylidene)hydrazinyl)-2-(quinolin-7-yloxy)pyrimidin-4-yl)morpholine (15)

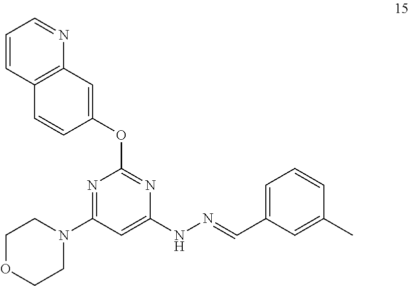 [SMILES: Cc1cccc(/C=N/Nc2cc(N3CCOCC3)nc(Oc3ccc4cccnc4c3)n2)c1]

Compound 15 was prepared in a similar manner as outlined in Example 1 by using quinolin-7-ol in step 1. ¹H NMR (400 MHz, CD₃OD) δ 8.87 (dd, J=4.4, 1.7 Hz, 1H), 8.42 (d, J=8.1 Hz, 1H), 8.01 (d, J=8.8 Hz, 1H), 7.90 (s, 1H), 7.79 (d, J=2.3 Hz, 1H), 7.58-7.44 (m, 4H), 7.28 (t, J=7.6 Hz, 1H), 7.19 (d, J=7.3 Hz, 1H), 6.25 (s, 1H), 3.78-3.64 (m, 4H), 3.54 (t, J=4.9 Hz, 4H), 2.39 (s, 3H). MS-ESI: m/z 441.24 observed (M+H)⁺.

### Example 8: Compound 16

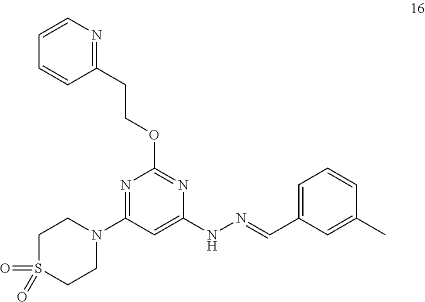 [SMILES: Cc1cccc(/C=N/Nc2cc(N3CCS(=O)(=O)CC3)nc(OCCc3ccccn3)n2)c1]

Compound 16 was prepared in a similar manner as outlined in Example 1. MS-ESI: m/z 467.26 observed (M+H)⁺.

### Example 9: (E)-4-(2-(2-(Pyridin-2-yl)ethoxy)-6-(2-(pyridin-4-ylmethylene)hydrazinyl)pyrimidin-4-yl)morpholine (17)

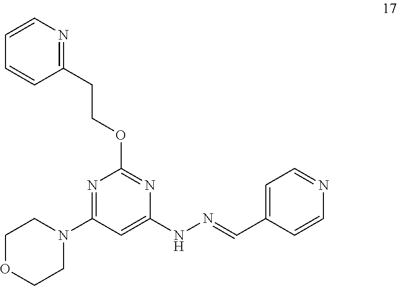 [SMILES: C(=N\Nc1cc(N2CCOCC2)nc(OCCc2ccccn2)n1)/c1ccncc1]

Compound 17 was prepared in a similar manner as outlined in Example 1 by using 2-(pyridin-2-yl)ethanol in step 1 and isonicotinaldehyde in step 4. MS-ESI: m/z 406.25 observed (M+H)⁺.

### Example 10: (E)-4-(2-(3-Methylbenzylidene)hydrazinyl)-6-morpholino-N-(pyridin-2-ylmethyl)pyrimidin-2-amine (18)

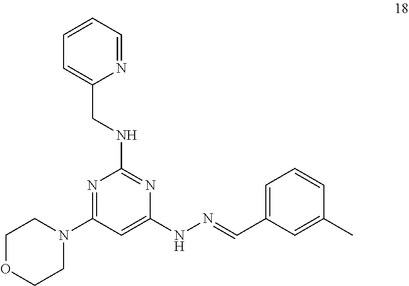 [SMILES: Cc1cccc(/C=N/Nc2cc(N3CCOCC3)nc(NCc3ccccn3)n2)c1]

Compound 18 was prepared in a similar manner as outlined in Example 1 by using pyridin-2-ylmethanamine in step 1. ¹H NMR (400 MHz, CD₃OD) δ 8.46 (d, J=5.1 Hz, 1H), 7.86 (s, 1H), 7.79 (td, J=7.6, 1.8 Hz, 1H), 7.53-7.38 (m, 3H), 7.31-7.21 (m, 2H), 7.17 (d, J=7.5 Hz, 1H), 5.87 (s, 1H), 4.65 (s, 2H), 3.70 (t, J=4.9 Hz, 4H), 3.55-3.39 (m, 4H), 2.38 (s, 3H). MS-ESI: m/z 404.25 observed (M+H)⁺.

### Example 11: (E)-4-(6-(2-((2-Methylpyridin-4-yl)methylene)hydrazinyl)-2-(2-(pyridin-2-yl)ethoxy)pyrimidin-4-yl)morpholine (19)

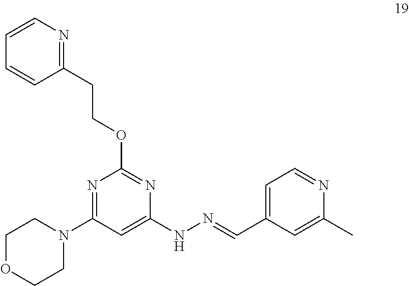 [SMILES: Cc1cc(/C=N/Nc2cc(N3CCOCC3)nc(OCCc3ccccn3)n2)ccn1]

Compound 19 was prepared in a similar manner as outlined in Example 1 by using 2-(pyridin-2-yl)ethanol in step 1 and 2-methylisonicotinaldehyde in step 4. MS-ESI: m/z 420.29 observed (M+H)⁺.

### Example 12: (E)-4-(6-(2-(3-Methylbenzylidene)hydrazinyl)-2-phenethoxypyrimidin-4-yl)morpholine (20)

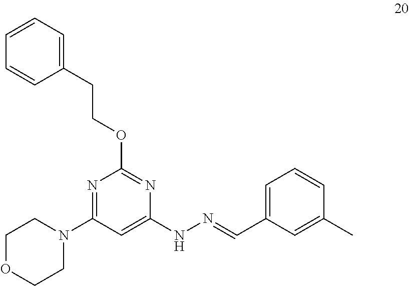 [SMILES: Cc1cccc(/C=N/Nc2cc(N3CCOCC3)nc(OCCc3ccccc3)n2)c1]

Compound 20 was prepared in a similar manner as outlined in Example 1 by using 2-phenylethanol in step 1. ¹H NMR (400 MHz, CD₃OD) δ 7.92 (s, 1H), 7.56-7.43 (m, 2H), 7.34-7.15 (m, 6H), 6.13 (s, 1H), 4.50 (t, J=7.1 Hz, 2H), 3.83-3.69 (m, 4H), 3.62 (t, J=4.9 Hz, 4H), 3.07 (t, J=7.1 Hz, 2H), 2.39 (s, 3H). MS-ESI: m/z 418.26 observed (M+H)⁺.

### Example 13: (E)-4-(2-(3-Methylbenzylidene)hydrazinyl)-6-(piperidin-1-yl)-2-(2-(pyridin-2-yl)ethoxy)pyrimidine (21)

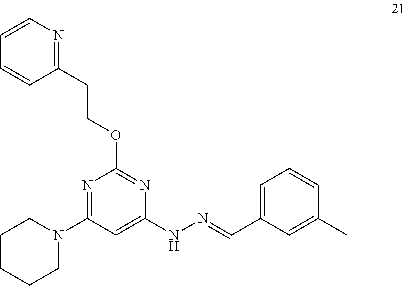 [SMILES: Cc1cccc(/C=N/Nc2cc(N3CCCCC3)nc(OCCc3ccccn3)n2)c1]

Compound 21 was prepared in a similar manner as outlined in Example 1 by using 2-(pyridin-2-yl)ethanol in step 1 and piperidine in step 2. MS-ESI: m/z 417.28 observed (M+H)⁺.

### Example 14: (E)-2,6-Dimethyl-4-(6-(2-(3-methylbenzylidene)hydrazinyl)-2-(2-(pyridin-2-yl)ethoxy)pyrimidin-4-yl)morpholine (22)

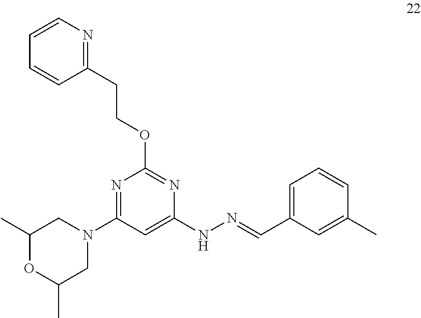

Compound 22 was prepared in a similar manner as outlined in Example 1 by using 2-(pyridin-2-yl)ethanol in step 1 and 2,6-dimethylmorpholine in step 2. MS-ESI: m/z 447.28 observed (M+H)⁺.

### Example 15: (E)-4-(6-(2-(3-Methylbenzylidene)hydrazinyl)-2-(2-(pyrimidin-2-yl)ethoxy)pyrimidin-4-yl)morpholine (23)

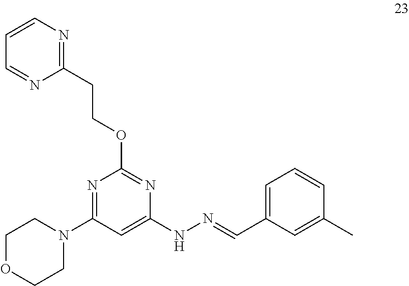 [SMILES: Cc1cccc(/C=N/Nc2cc(N3CCOCC3)nc(OCCc3ncccn3)n2)c1]

Compound 23 was prepared in a similar manner as outlined in Example 1 by using 2-(pyrimidin-2-yl)ethanol in step 1. ¹H NMR (400 MHz, CD₃OD) δ 8.75 (dd, J=5.0, 1.6 Hz, 2H), 7.90 (d, J=1.5 Hz, 1H), 7.55-7.45 (m, 2H), 7.38 (td, J=4.9, 1.5 Hz, 1H), 7.28 (td, J=7.7, 1.4 Hz, 1H), 7.19 (d, J=7.7 Hz, 1H), 6.16-6.07 (m, 1H), 4.81 (td, J=6.6, 1.6 Hz, 2H), 3.81-3.73 (m, 4H), 3.64-3.56 (m, 4H), 3.41 (td, J=6.6, 1.5 Hz, 2H), 2.43-2.34 (m, 3H). MS-ESI: m/z 420.22 observed (M+H)⁺.

### Example 16: (E)-6-(2-(3-Methylbenzylidene)hydrazinyl)-N,N-dipropyl-2-(2-(pyrimidin-2-yl)ethoxy)pyrimidin-4-amine (24)

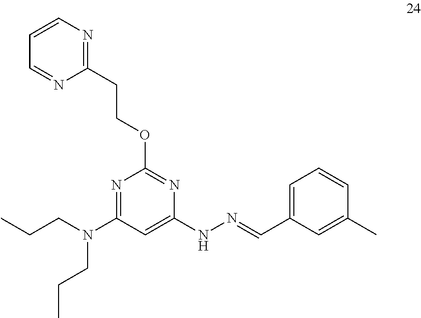 [SMILES: CCCN(CCC)c1cc(N/N=C/c2cccc(C)c2)nc(OCCc2ncccn2)n1]

Compound 24 was prepared in a similar manner as outlined in Example 1 by using 2-(pyridin-2-yl)ethanol in step 1 and dipropylamine in step 2. MS-ESI: m/z 433.34 observed (M+H)⁺.

### Example 17: (E)-4-(6-(2-(3-Methylbenzylidene)hydrazinyl)-2-(pyridin-2-yloxy)pyrimidin-4-yl)morpholine (25)

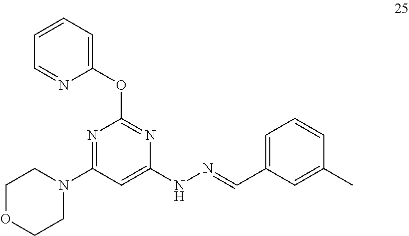 [SMILES: Cc1cccc(/C=N/Nc2cc(N3CCOCC3)nc(Oc3ccccn3)n2)c1]

Compound 25 was prepared in a similar manner as outlined in Example 1 by using 2-hydroxypyridine in step 1. MS-ESI: m/z 391.23 observed (M+H)⁺.

### Example 18: (E)-4-(6-(2-(3-Methylbenzylidene)hydrazinyl)-2-(2-(pyridin-2-yl)ethoxy)pyrimidin-4-yl)thiomorpholine (26)

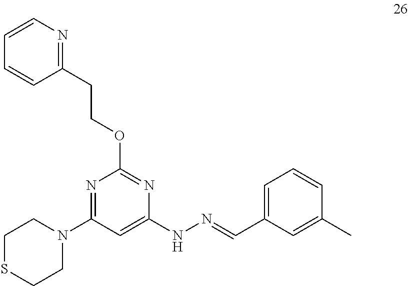 [SMILES: Cc1cccc(/C=N/Nc2cc(N3CCSCC3)nc(OCCc3ccccn3)n2)c1]

Compound 26 was prepared in a similar manner as outlined in Example 1 by using 2-(pyridin-2-yl)ethanol in step 1 and thiomorpholine in step 2. MS-ESI: m/z 435.23 observed (M+H)⁺.

### Example 19: (E)-3-(4-(2-(3-Methylbenzylidene)hydrazinyl)-6-morpholinopyrimidin-2-yl)phenol (27)

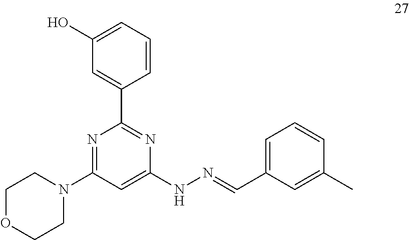 [SMILES: Cc1cccc(/C=N/Nc2cc(N3CCOCC3)nc(-c3cccc(O)c3)n2)c1]

Compound 27 was prepared in a similar manner as outlined in Example 2. MS-ESI: m/z 390.28 observed (M+H)⁺.

### Example 20: (E)-4-(6-(2-(3-Methylbenzylidene)hydrazinyl)-2-(1-(pyridin-2-yl)propan-2-yloxy)pyrimidin-4-yl)morpholine (28)

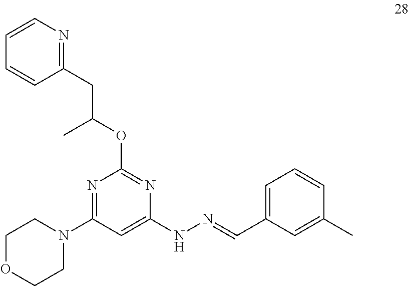 [SMILES: Cc1cccc(/C=N/Nc2cc(N3CCOCC3)nc(OC(C)Cc3ccccn3)n2)c1]

Compound 28 was prepared in a similar manner as outlined in Example 1 by using 1-(pyridin-2-yl)propan-2-ol in step 1. MS-ESI: m/z 433.28 observed (M+H)⁺.

### Example 21: (E)-4-(6-(2-(3-Methylbenzylidene)hydrazinyl)-2-(2-(pyrimidin-4-yl)ethoxy)pyrimidin-4-yl)morpholine (29)

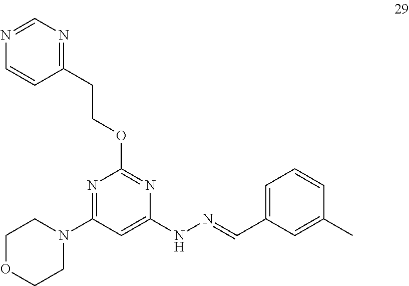 [SMILES: Cc1cccc(/C=N/Nc2cc(N3CCOCC3)nc(OCCc3ccncn3)n2)c1]

Compound 29 was prepared in a similar manner as outlined in Example 1 by using 2-(pyrimidin-4-yl)ethanol in step 1. MS-ESI: m/z 420.32 observed (M+H)⁺.

### Example 22: (E)-4-(6-(2-(3-Methylbenzylidene)hydrazinyl)-2-(3-(pyridin-2-yl)phenyl)pyrimidin-4-yl)morpholine (30)

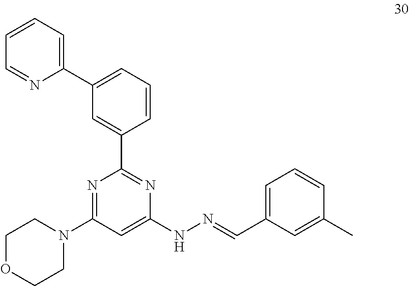 [SMILES: Cc1cccc(/C=N/Nc2cc(N3CCOCC3)nc(-c3cccc(-c4ccccn4)c3)n2)c1]

Compound 30 was prepared in a similar manner as outlined in Example 2. MS-ESI: m/z 451.3 observed (M+H)⁺.

### Example 23: (E)-4-(5-Methoxy-6-(2-(3-methylbenzylidene)hydrazinyl)-2-(2-(pyridin-2-yl)ethoxy)pyrimidin-4-yl)morpholine (31)

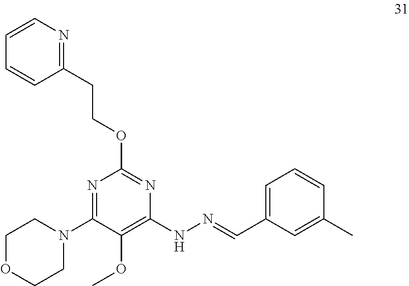 [SMILES: COc1c(N/N=C/c2cccc(C)c2)nc(OCCc2ccccn2)nc1N1CCOCC1]

Compound 31 was prepared in a similar manner as outlined in Example 1 by using 2-(pyridin-2-yl)ethanol and 4,6-dichloro-5-methoxypyrimidin-2-yl methanesulfonate in step 1. MS-ESI: m/z 449.34 observed (M+H)⁺.

### Example 24: (E)-4-(5-Methyl-6-(2-(3-methylbenzylidene)hydrazinyl)-2-(2-(pyridin-2-yl)ethoxy)pyrimidin-4-yl)morpholine (32)

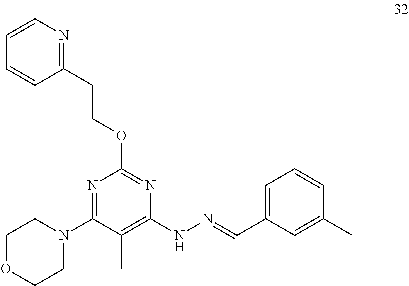 [SMILES: Cc1cccc(/C=N/Nc2nc(OCCc3ccccn3)nc(N3CCOCC3)c2C)c1]

Compound 32 was prepared in a similar manner as outlined in Example 1 by using 2-(pyridin-2-yl)ethanol and 4,6-dichloro-5-methylpyrimidin-2-yl methanesulfonate in step 1. MS-ESI: m/z 449.34 observed (M+H)⁺.

### Example 25: (E)-4-(2-(2-(Pyridin-2-yl)ethoxy)-6-(2-(3-(trifluoromethyl)benzylidene)hydrazinyl)pyrimidin-4-yl)morpholine (33)

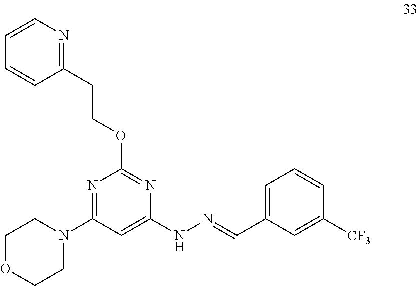 [SMILES: FC(F)(F)c1cccc(/C=N/Nc2cc(N3CCOCC3)nc(OCCc3ccccn3)n2)c1]

Compound 33 was prepared in a similar manner as outlined in Example 1 by using 2-(pyridin-2-yl)ethanol in step 1 and 3-trifluoromethylbenzaldehyde in step 4. ¹H NMR (400 MHz, DMSO-d₆) δ 11.08 (s, 1H), 8.51 (d, J=4.8 Hz, 1H), 8.08 (d, J=14.6 Hz, 2H), 7.95 (s, 1H), 7.72 (dd, J=12.8, 7.0 Hz, 2H), 7.65 (d, J=7.7 Hz, 1H), 7.34 (d, J=7.8 Hz, 1H), 7.28-7.21 (m, 1H), 6.11 (s, 1H), 4.55 (t, J=6.8 Hz, 2H), 3.67 (t, J=4.7 Hz, 4H), 3.54 (t, J=4.7 Hz, 4H), 3.15 (t, J=6.7 Hz, 2H). MS-ESI: m/z 473.16 observed (M+H)⁺.

### Example 26: (E)-4-(2-(2-(Pyridin-2-yl)ethoxy)-6-(2-(1-m-tolylethylidene)hydrazinyl)pyrimidin-4-yl)morpholine (34)

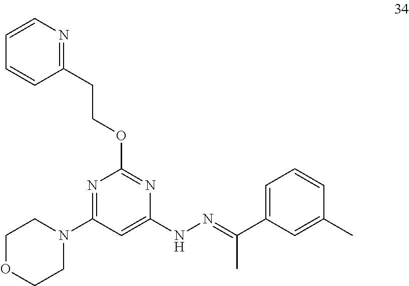 [SMILES: C/C(=N\Nc1cc(N2CCOCC2)nc(OCCc2ccccn2)n1)c1cccc(C)c1]

Compound 34 was prepared in a similar manner as outlined in Example 1 by using 2-(pyridin-2-yl)ethanol in step 1 and 1-m-tolylethanone in step 4. ¹H NMR (400 MHz, CD₃OD) δ 8.65 (ddd, J=5.4, 1.8, 0.9 Hz, 1H), 8.19 (t, J=7.8 Hz, 1H), 7.79 (d, J=7.9 Hz, 1H), 7.73 (s, 1H), 7.65 (t, J=7.3 Hz, 2H), 7.32 (t, J=7.6 Hz, 1H), 7.26 (d, J=7.6 Hz, 1H), 6.00 (s, 1H), 3.85-3.65 (m, 8H), 3.46 (t, J=6.2 Hz, 2H), 2.42 (s, 3H), 2.39 (s, 3H). MS-ESI: m/z 433.28 observed (M+H)⁺.

### Example 27: (E)-4-(6-(2-(3-Methylbenzylidene)hydrazinyl)-2-(2-(pyridin-2-yl)ethoxy)pyrimidin-4-yl)-1,4-oxazepane (35)

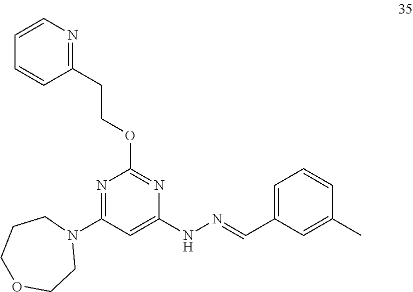 [SMILES: Cc1cccc(/C=N/Nc2cc(N3CCCOCC3)nc(OCCc3ccccn3)n2)c1]

Compound 35 was prepared in a similar manner as outlined in Example 1 by using 2-(pyridin-2-yl)ethanol in step 1 and 1,4-oxazepane in step 2. ¹H NMR (400 MHz, CD₃OD) δ 8.70 (d, J=5.6 Hz, 1H), 8.27 (t, J=7.8 Hz, 1H), 8.05 (d, J=1.8 Hz, 1H), 7.87 (d, J=8.0 Hz, 1H), 7.71 (dd, J=13.7, 4.8 Hz, 2H), 7.63-7.56 (m, 1H), 7.37-7.31 (m, 1H), 7.31-7.25 (m, 1H), 5.78 (s, 1H), 4.04-3.69 (m, 8H), 3.58-3.46 (m, 2H), 2.41 (s, 3H), 2.14-1.92 (m, 2H) [one peak under water peak]. MS-ESI: m/z 433.28 observed (M+H)⁺.

### Example 28: (E)-4-(2-(2-Methyl-2-phenylpropoxy)-6-(2-(3-methylbenzylidene)hydrazinyl)pyrimidin-4-yl)morpholine (36)

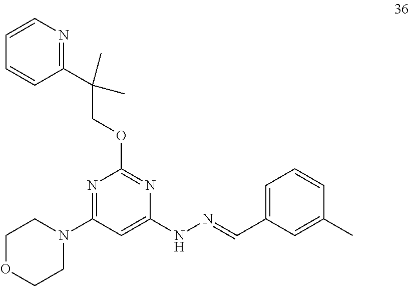 [SMILES: Cc1cccc(/C=N/Nc2cc(N3CCOCC3)nc(OCC(C)(C)c3ccccn3)n2)c1]

Compound 36 was prepared in a similar manner as outlined in Example 1 by using 2-methyl-2-phenylpropan-1-ol in step 1. MS-ESI: m/z 446.30 observed (M+H)⁺.

### Example 29: (E)-4-(6-(1-Methyl-2-(3-methylbenzylidene)hydrazinyl)-2-(2-(pyridin-2-yl)ethoxy)pyrimidin-4-yl)morpholine (37)

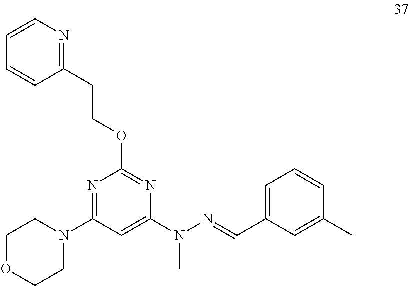 [SMILES: Cc1cccc(/C=N/N(C)c2cc(N3CCOCC3)nc(OCCc3ccccn3)n2)c1]

Compound 37 was prepared in a similar manner as outlined in Example 1 by using 2-(pyridin-2-yl)ethanol in step 1 and methylhydrazine in step 3. MS-ESI: m/z 433.34 observed (M+H)⁺.

### Example 30: (E)-4-(2-(3-Methylbenzylidene)hydrazinyl)-6-morpholino-2-(2-(pyridin-2-yl)ethoxy)pyrimidine-5-carbonitrile (38)

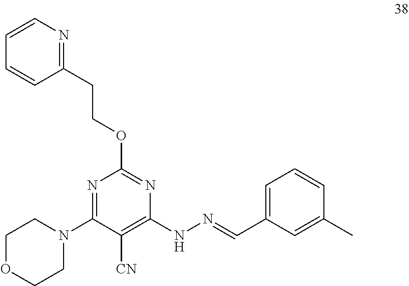 [SMILES: Cc1cccc(/C=N/Nc2nc(OCCc3ccccn3)nc(N3CCOCC3)c2C#N)c1]

Compound 38 was prepared in a similar manner as outlined in Example 1 by using 2-(pyridin-2-yl)ethanol and 4,6-dichloro-5-cyanopyrimidin-2-yl methanesulfonate in step 1. MS-ESI: m/z 444.34 observed (M+H)⁺.

### Example 31: (E)-4-(5-Chloro-6-(2-(3-methylbenzylidene)hydrazinyl)-2-(2-(pyridin-2-yl)ethoxy)pyrimidin-4-yl)morpholine (39)

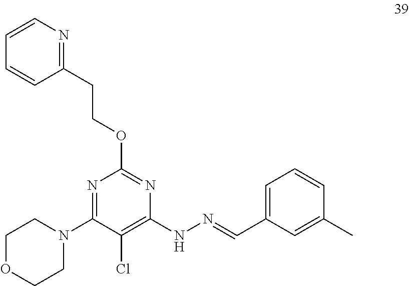 [SMILES: Cc1cccc(/C=N/Nc2nc(OCCc3ccccn3)nc(N3CCOCC3)c2Cl)c1]

Compound 39 was prepared in a similar manner as outlined in Example 1 by using 2-(pyridin-2-yl)ethanol and 4,6-dichloro-5-chloropyrimidin-2-yl methanesulfonate in step 1. MS-ESI: m/z 453.25 observed (M+H)⁺.

### Example 32: (E)-4-(5-Fluoro-6-(2-(3-methylbenzylidene)hydrazinyl)-2-(2-(pyridin-2-yl)ethoxy)pyrimidin-4-yl)morpholine (40)

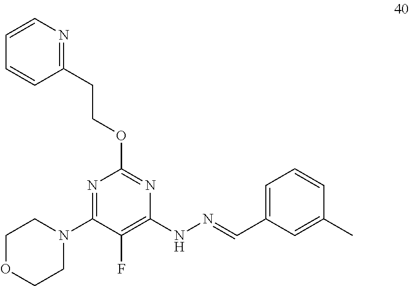 [SMILES: Cc1cccc(/C=N/Nc2nc(OCCc3ccccn3)nc(N3CCOCC3)c2F)c1]

Compound 40 was prepared in a similar manner as outlined in Example 1 by using 2-(pyridin-2-yl)ethanol and 4,6-dichloro-5-fluoropyrimidin-2-yl methanesulfonate in step 1. MS-ESI: m/z 437.26 observed (M+H)⁺.

### Example 33: (E)-4-(2-(3-Methylbenzylidene)hydrazinyl)-2-(2-(pyridin-2-yl)ethoxy)-6-(tetrahydro-2H-pyran-4-yl)pyrimidine (41)

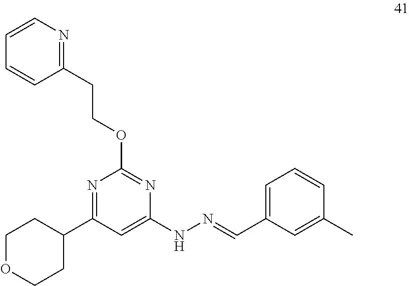 [SMILES: Cc1cccc(/C=N/Nc2cc(C3CCOCC3)nc(OCCc3ccccn3)n2)c1]

Compound 41 was prepared in a similar manner as outlined in Example 2. ¹H NMR (400 MHz, CD₃OD) δ 8.64 (dt, J=5.3, 1.2 Hz, 1H), 8.22 (s, 1H), 8.16 (td, J=7.8, 1.7 Hz, 1H), 7.76 (d, J=6.9 Hz, 2H), 7.68 (d, J=7.3 Hz, 1H), 7.65-7.59 (m, 1H), 7.35 (dt, J=13.9, 7.5 Hz, 2H), 6.42 (s, 1H), 4.12-3.99 (m, 2H), 3.56 (td, J=11.8, 2.1 Hz, 2H), 3.46 (t, J=6.3 Hz, 2H), 3.23-3.10 (m, 1H), 2.42 (s, 3H), 1.95-1.85 (m, 2H), 1.86-1.71 (m, 2H) [one peak under water peak]. MS-ESI: m/z 418.29 observed (M+H)⁺.

### Example 34: (E)-4-(2-(3-Methylbenzylidene)hydrazinyl)-6-(4-methylpiperazin-1-yl)-2-(2-(pyridin-2-yl)ethoxy)pyrimidine (42)

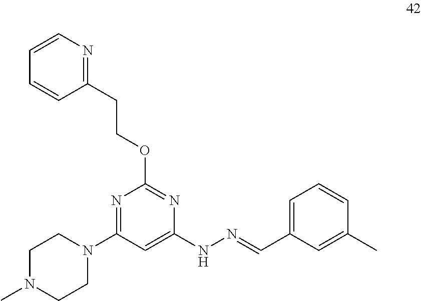

Compound 42 was prepared in a similar manner as outlined in Example 1 by using 2-(pyridin-2-yl)ethanol in step 1 and N-methylpiperazine in step 2. ¹H NMR (400 MHz, CD₃OD) δ 8.70 (dt, J=5.4, 1.4 Hz, 1H), 8.36 (t, J=7.7 Hz, 1H), 7.99 (s, 1H), 7.93 (d, J=8.0 Hz, 1H), 7.79 (t, J=6.7 Hz, 1H), 7.58 (s, 1H), 7.53 (d, J=7.7 Hz, 1H), 7.29 (t, J=7.6 Hz, 1H), 7.22 (d, J=7.6 Hz, 1H), 6.16 (s, 1H), 4.78 (t, J=6.0 Hz, 2H), 3.53-3.44 (m, 2H), 2.97 (s, 3H), 2.38 (s, 3H). MS-ESI: m/z 432.33 observed (M+H)⁺.

### Example 35: (E)-5-(2-(4-(2-(3-Methylbenzylidene)hydrazinyl)-6-morpholinopyrimidin-2-yloxy)ethyl)pyridin-2-amine (43)

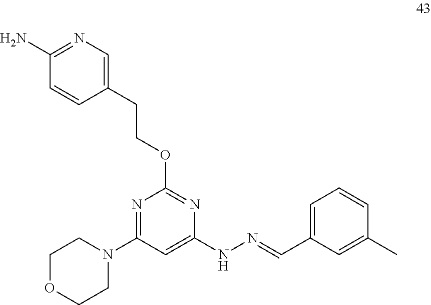 [SMILES: Cc1cccc(/C=N/Nc2cc(N3CCOCC3)nc(OCCc3ccc(N)nc3)n2)c1]

Compound 43 was prepared in a similar manner as outlined in Example 1 by using 2-(6-aminopyridin-3-yl)ethanol in step 1. MS-ESI: m/z 434.36 observed (M+H)⁺.

### Example 36: (E)-4-(6-(2-(3-Methylbenzylidene)hydrazinyl)-2-(2-(pyrazin-2-yl)ethoxy)pyrimidin-4-yl)morpholine (44)

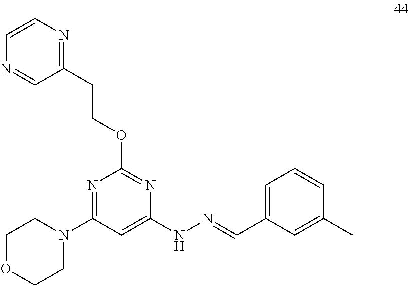 [SMILES: Cc1cccc(/C=N/Nc2cc(N3CCOCC3)nc(OCCc3cnccn3)n2)c1]

Compound 44 was prepared in a similar manner as outlined in Example 1 by using 2-(pyrazin-2-yl)ethanol in step 1. MS-ESI: m/z 420.32 observed (M+H)⁺.

### Example 37: (E)-4-(2-(2,2-Difluoro-2-phenylethoxy)-6-(2-(3-methylbenzylidene)hydrazinyl)pyrimidin-4-yl)morpholine (45)

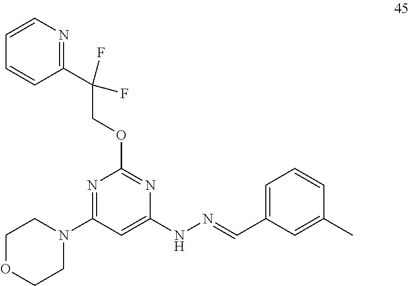 [SMILES: Cc1cccc(/C=N/Nc2cc(N3CCOCC3)nc(OCC(F)(F)c3ccccn3)n2)c1]

Compound 45 was prepared in a similar manner as outlined in Example 1 by using 2,2-difluoro-2-phenylethanol in step 1. MS-ESI: m/z 454.27 observed (M+H)⁺.

### Example 38: (E)-4-(6-(2-Benzylidenehydrazinyl)-2-(2-(pyridin-2-yl)ethoxy)pyrimidin-4-yl)morpholine (46)

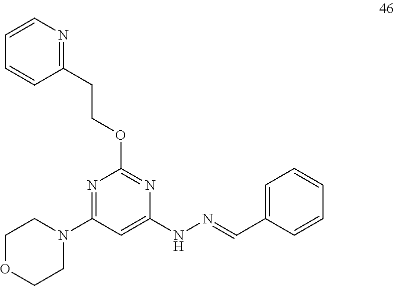 [SMILES: C(=N\Nc1cc(N2CCOCC2)nc(OCCc2ccccn2)n1)/c1ccccc1]

Compound 46 was prepared in a similar manner as outlined in Example 1 by using 2-(pyridin-2-yl)ethanol in step 1 and benzaldehyde in step 4. ¹H NMR (400 MHz, DMSO-d₆) δ 10.90 (s, 1H), 8.51 (ddd, J=4.9, 1.9, 0.9 Hz, 1H), 8.02 (s, 1H), 7.76-7.66 (m, 3H), 7.45-7.31 (m, 4H), 7.24 (ddd, J=7.6, 4.9, 1.2 Hz, 1H), 6.08 (s, 1H), 4.55 (t, J=6.8 Hz, 2H), 3.67 (t, J=4.7 Hz, 4H), 3.53 (t, J=4.9 Hz, 4H), 3.15 (t, J=6.7 Hz, 2H). MS-ESI: m/z 405.20 observed (M+H)⁺.

### Example 39: (E)-4-(2-(Benzyloxy)-6-(2-(3-methylbenzylidene)hydrazinyl)pyrimidin-4-yl)morpholine (47)

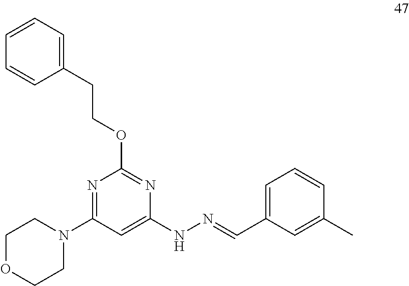 [SMILES: Cc1cccc(/C=N/Nc2cc(N3CCOCC3)nc(OCCc3ccccc3)n2)c1]

Compound 47 was prepared in a similar manner as outlined in Example 1 by using benzyl alcohol in step 1. MS-ESI: m/z 404.32 observed (M+H)⁺.

### Example 40: (E)-3-((2-(6-Morpholino-2-(2-(pyridin-2-yl)ethoxy)pyrimidin-4-yl)hydrazono)methyl)benzonitrile (48)

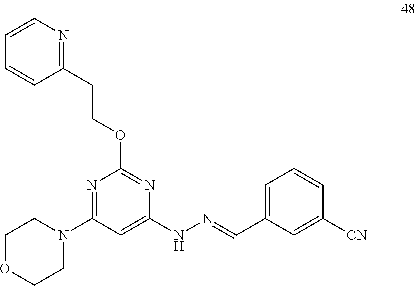 [SMILES: N#Cc1cccc(/C=N/Nc2cc(N3CCOCC3)nc(OCCc3ccccn3)n2)c1]

Compound 48 was prepared in a similar manner as outlined in Example 1 by using 2-(pyridin-2-yl)ethanol in step 1 and 3-cyanobenzaldehyde in step 4. ¹H NMR (400 MHz, CDCl₃) δ 13.47 (s, 1H), 8.79 (d, J=5.4 Hz, 1H), 8.37 (td, J=7.8, 1.4 Hz, 1H), 8.24-8.09 (m, 3H), 7.82 (t, J=7.1 Hz, 2H), 7.69 (dt, J=7.8, 1.4 Hz, 1H), 7.55 (t, J=7.8 Hz, 1H), 6.06 (s, 1H), 4.80 (t, J=5.5 Hz, 2H), 3.86 (br s, 8H), 3.70 (t, J=5.5 Hz, 2H). MS-ESI: m/z 430.17 observed (M+H)⁺.

### Example 41: (E)-4-(6-(2-(3-Methylbenzylidene)hydrazinyl)-2-(1-(pyrimidin-2-yl)ethoxy)pyrimidin-4-yl)morpholine (49)

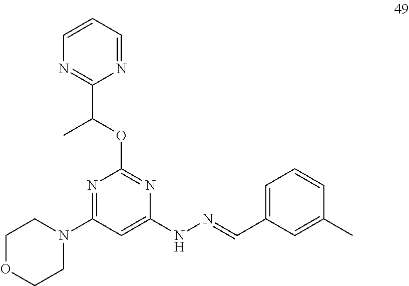 [SMILES: Cc1cccc(/C=N/Nc2cc(N3CCOCC3)nc(OC(C)c3ncccn3)n2)c1]

Compound 49 was prepared in a similar manner as outlined in Example 1 by using 1-(pyrimidin-2-yl)ethanol in step 1. MS-ESI: m/z 420.25 observed (M+H)⁺.

### Example 42: (E)-4-(6-(2-(3-Methylbenzylidene)hydrazinyl)-2-((4-methylpyrimidin-2-yl)methoxy)pyrimidin-4-yl)morpholine (50)

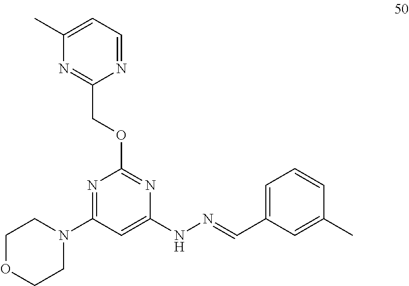 [SMILES: Cc1cccc(/C=N/Nc2cc(N3CCOCC3)nc(OCc3nccc(C)n3)n2)c1]

Compound 50 was prepared in a similar manner as outlined in Example 1 by using (4-methylpyrimidin-2-yl)methanol in step 1. MS-ESI: m/z 420.25 observed (M+H)⁺.

### Example 43: (E)-4-(6-(2-(3-Methylbenzylidene)hydrazinyl)-2-phenoxypyrimidin-4-yl)morpholine (51)

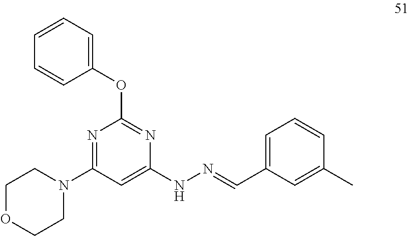 [SMILES: Cc1cccc(/C=N/Nc2cc(N3CCOCC3)nc(Oc3ccccc3)n2)c1]

Compound 51 was prepared in a similar manner as outlined in Example 1 by using phenol in step 1. MS-ESI: m/z 390.28 observed (M+H)⁺.

### Example 44: (E)-4-(2-((5-Chloropyrimidin-2-yl)methoxy)-6-(2-(3-methylbenzylidene)hydrazinyl)pyrimidin-4-yl)morpholine (52)

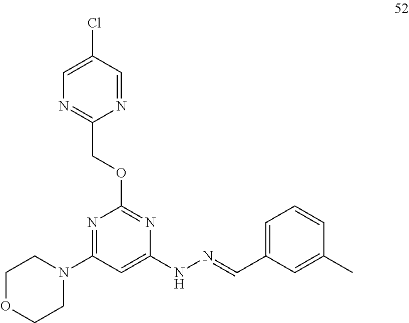 [SMILES: Cc1cccc(/C=N/Nc2cc(N3CCOCC3)nc(OCc3ncc(Cl)cn3)n2)c1]

Compound 52 was prepared in a similar manner as outlined in Example 1 by using (5-chloropyrimidin-2-yl)methanol in step 1. MS-ESI: m/z 440.23 observed (M+H)⁺.

### Example 45: (E)-4-(6-(2-(3-Methylbenzylidene)hydrazinyl)-2-(naphthalen-2-yloxy)pyrimidin-4-yl)morpholine (53)

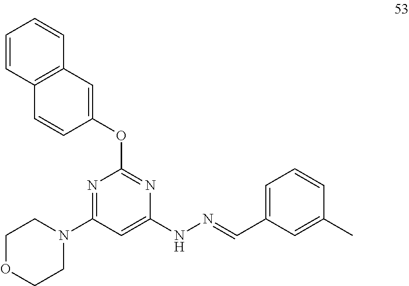 [SMILES: Cc1cccc(/C=N/Nc2cc(N3CCOCC3)nc(Oc3ccc4ccccc4c3)n2)c1]

Compound 53 was prepared in a similar manner as outlined in Example 1 by using naphthalen-2-ol in step 1. ¹H NMR (400 MHz, DMSO-d₆) δ 10.91 (s, 1H), 8.01-7.86 (m, 4H), 7.67 (d, J=2.3 Hz, 1H), 7.57-7.43 (m, 4H), 7.36 (dd, J=8.8, 2.4 Hz, 1H), 7.29 (t, J=7.6 Hz, 1H), 7.18 (d, J=7.5 Hz, 1H), 6.17 (s, 1H), 3.69-3.61 (m, 4H), 3.55-3.46 (m, 4H), 2.34 (s, 3H). MS-ESI: m/z 440.30 observed (M+H)⁺.

### Example 46: (E)-4-(6-(2-(3-Methylbenzylidene)hydrazinyl)-2-((4-(trifluoromethyl)pyrimidin-2-yl)methoxy)pyrimidin-4-yl)morpholine (54)

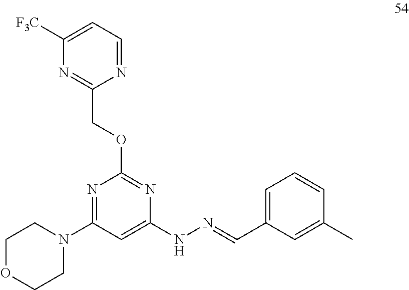 [SMILES: Cc1cccc(/C=N/Nc2cc(N3CCOCC3)nc(OCc3nccc(C(F)(F)F)n3)n2)c1]

Compound 54 was prepared in a similar manner as outlined in Example 1 by using (4-trifluoromethylpyrimidin-2-yl)methanol in step 1. MS-ESI: m/z 474.31 observed (M+H)⁺.

### Example 47: (E)-4-(2-(2-Chloroquinolin-6-yloxy)-6-(2-(3-methylbenzylidene)hydrazinyl)pyrimidin-4-yl)morpholine (55)

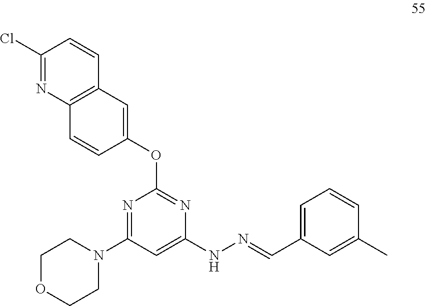 [SMILES: Cc1cccc(/C=N/Nc2cc(N3CCOCC3)nc(Oc3ccc4nc(Cl)ccc4c3)n2)c1]

Compound 55 was prepared in a similar manner as outlined in Example 1 by using (4-chloropyrimidin-2-yl)methanol in step 1. MS-ESI: m/z 475.25 observed (M+H)⁺.

### Example 48: (E)-4-(6-(2-(4-Methylbenzylidene)hydrazinyl)-2-(2-(pyridin-2-yl)ethoxy)pyrimidin-4-yl)morpholine (56)

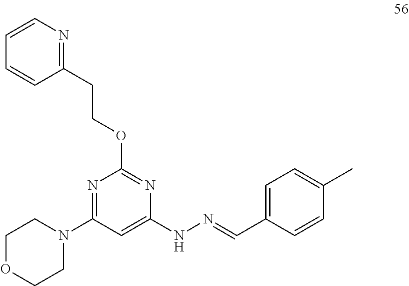 [SMILES: Cc1ccc(/C=N/Nc2cc(N3CCOCC3)nc(OCCc3ccccn3)n2)cc1]

Compound 56 was prepared in a similar manner as outlined in Example 1 by using 2-(pyridin-2-yl)ethanol in step 1 and 4-methylbenzaldehyde in step 4. ¹H NMR (400 MHz, DMSO-d₆) δ 10.89 (s, 1H), 8.61 (dd, J=5.3, 1.8 Hz, 1H), 8.01 (s, 1H), 7.95 (t, J=7.6 Hz, 1H), 7.59 (d, J=7.8 Hz, 2H), 7.53 (d, J=7.8 Hz, 1H), 7.43 (dd, J=7.4, 5.2 Hz, 1H), 7.22 (d, J=7.8 Hz, 2H), 6.05 (s, 1H), 4.58 (t, J=6.5 Hz, 2H), 3.67 (t, J=4.9 Hz, 4H), 3.52 (t, J=4.9 Hz, 4H), 3.25 (t, J=6.5 Hz, 2H), 2.33 (s, 3H). MS-ESI: m/z 419.31 observed (M+H)⁺.

### Example 49: (E)-4-(2-((4-Chloropyrimidin-2-yl)methoxy)-6-(2-(3-methylbenzylidene)hydrazinyl)pyrimidin-4-yl)morpholine (57)

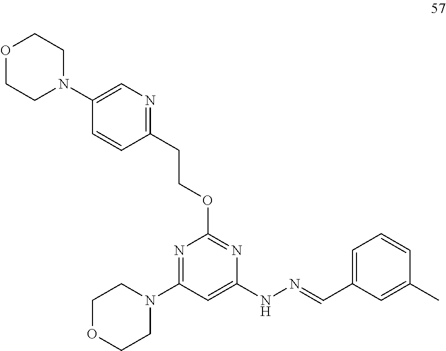 [SMILES: Cc1cccc(/C=N/Nc2cc(N3CCOCC3)nc(OCCc3ccc(N4CCOCC4)cn3)n2)c1]

Compound 57 was prepared in a similar manner as outlined in Example 1 by using 2-(5-morpholinopyridin-2-yl)ethanol in step 1. MS-ESI: m/z 504.41 observed (M+H)⁺.

### Example 50: (E)-4-(6-(2-(3-Methylbenzylidene)hydrazinyl)-2-((5-methylpyrimidin-2-yl)methoxy)pyrimidin-4-yl)morpholine (58)

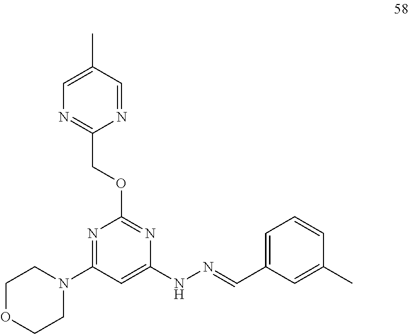 [SMILES: Cc1cnc(COc2nc(N/N=C/c3cccc(C)c3)cc(N3CCOCC3)n2)nc1]

Compound 58 was prepared in a similar manner as outlined in Example 1 by using (5-methylpyrimidin-2-yl)methanol in step 1. MS-ESI: m/z 420.25 observed (M+H)⁺.

### Example 51: (E)-4-(2-((4,6-Dimethylpyrimidin-2-yl)methoxy)-6-(2-(3-methylbenzylidene)hydrazinyl)pyrimidin-4-yl)morpholine (59)

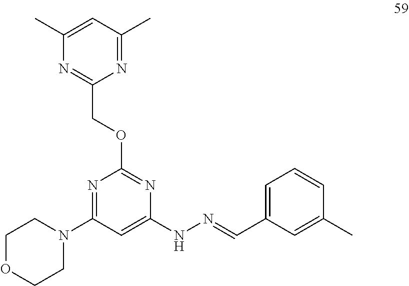 [SMILES: Cc1cccc(/C=N/Nc2cc(N3CCOCC3)nc(OCc3nc(C)cc(C)n3)n2)c1]

Compound 59 was prepared in a similar manner as outlined in Example 1 by using (4,6-dimethylpyrimidin-2-yl)methanol in step 1. MS-ESI: m/z 434.29 observed (M+H)⁺.

### Example 52: (E)-4-(2-((5-Methoxypyrimidin-2-yl)methoxy)-6-(2-(3-methylbenzylidene)hydrazinyl)pyrimidin-4-yl)morpholine (60)

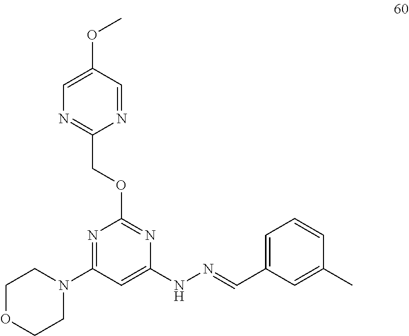 [SMILES: COc1cnc(COc2nc(N/N=C/c3cccc(C)c3)cc(N3CCOCC3)n2)nc1]

Compound 60 was prepared in a similar manner as outlined in Example 1 by using (5-methoxypyrimidin-2-yl)methanol in step 1. MS-ESI: m/z 436.25 observed (M+H)⁺.

### Example 53: (E)-4-(6-(2-(3-(Methylsulfonyl)benzylidene)hydrazinyl)-2-(2-(pyridin-2-yl)ethoxy)pyrimidin-4-yl)morpholine (61)

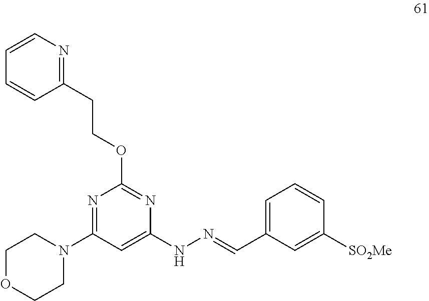 [SMILES: CS(=O)(=O)c1cccc(/C=N/Nc2cc(N3CCOCC3)nc(OCCc3ccccn3)n2)c1]

Compound 61 was prepared in a similar manner as outlined in Example 1 by using 2-(pyridin-2-yl)ethanol in step 1 and 3-(methylsulfonyl)benzaldehyde in step 4. ¹H NMR (400 MHz, DMSO-d₆) δ 11.23 (s, 1H), 8.81-8.75 (m, 1H), 8.33 (td, J=7.8, 1.7 Hz, 1H), 8.18-8.09 (m, 3H), 7.93-7.84 (m, 2H), 7.76 (ddd, J=7.4, 5.7, 1.2 Hz, 1H), 7.69 (t, J=7.8 Hz, 1H), 6.09 (s, 1H), 4.64 (t, J=6.2 Hz, 2H), 3.72-3.64 (m, 4H), 3.59-3.51 (m, 4H), 3.39 (t, J=6.2 Hz, 2H), 3.28 (s, 3H). MS-ESI: m/z 483.22 observed (M+H)⁺.

### Example 54: (E)-4-(6-(2-(3-Methylbenzylidene)hydrazinyl)-2-(2-methylquinolin-6-yloxy)pyrimidin-4-yl)morpholine (62)

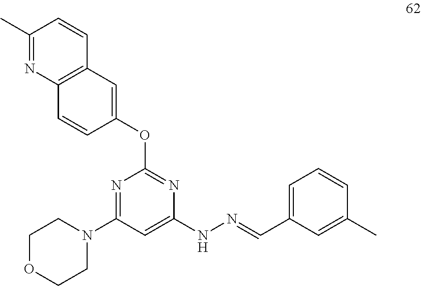

Compound 62 was prepared in a similar manner as outlined in Example 1 by using 2-methylquinolin-6-ol in step 1. MS-ESI: m/z 455.28 observed (M+H)⁺.

### Example 55: (E)-4-(2-(4-Chloroquinolin-6-yloxy)-6-(2-(3-methylbenzylidene)hydrazinyl)pyrimidin-4-yl)morpholine (63)

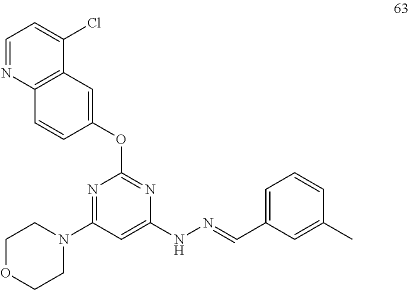 [SMILES: Cc1cccc(/C=N/Nc2cc(N3CCOCC3)nc(Oc3ccc4nccc(Cl)c4c3)n2)c1]

Compound 63 was prepared in a similar manner as outlined in Example 1 by using 4-chloroquinolin-6-ol in step 1. MS-ESI: m/z 475.19 observed (M+H)⁺.

### Example 56: (E)-2-((4-(2-(3-Methylbenzylidene)hydrazinyl)-6-morpholinopyrimidin-2-yloxy)methyl)quinazolin-4(3H)-one (64)

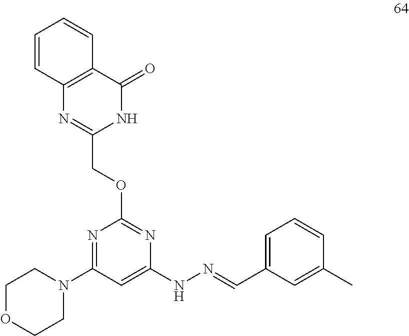 [SMILES: Cc1cccc(/C=N/Nc2cc(N3CCOCC3)nc(OCc3nc4ccccc4c(=O)[nH]3)n2)c1]

Compound 64 was prepared in a similar manner as outlined in Example 1 by using 2-(hydroxymethyl)quinazolin-4(3H)-one in step 1. MS-ESI: m/z 472.29 observed (M+H)⁺.

### Example 57: (E)-4-(6-(2-(3-Methylbenzylidene)hydrazinyl)-2-(1-(pyrimidin-2-yl)propoxy)pyrimidin-4-yl)morpholine (65)

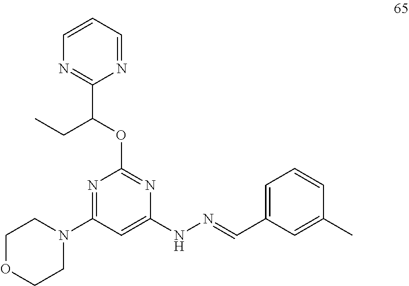 [SMILES: CCC(Oc1nc(N/N=C/c2cccc(C)c2)cc(N2CCOCC2)n1)c1ncccn1]

Compound 65 was prepared in a similar manner as outlined in Example 1 by using 1-(pyrimidin-2-yl)propan-1-ol in step 1. MS-ESI: m/z 434.15 observed (M+H)⁺.

### Example 58: (E)-4-(2-(3-Chloroquinolin-6-yloxy)-6-(2-(3-methylbenzylidene)hydrazinyl)pyrimidin-4-yl)morpholine (66)

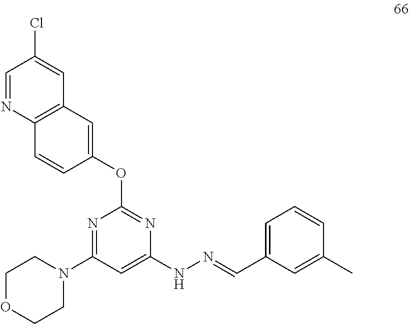 [SMILES: Cc1cccc(/C=N/Nc2cc(N3CCOCC3)nc(Oc3ccc4ncc(Cl)cc4c3)n2)c1]

Compound 66 was prepared in a similar manner as outlined in Example 1 by using 3-chloroquinolin-6-ol in step 1. MS-ESI: m/z 475.12 observed (M+H)⁺.

### Example 59: (E)-4-(2-(3-Fluoroquinolin-6-yloxy)-6-(2-(3-methylbenzylidene)hydrazinyl)pyrimidin-4-yl)morpholine (67)

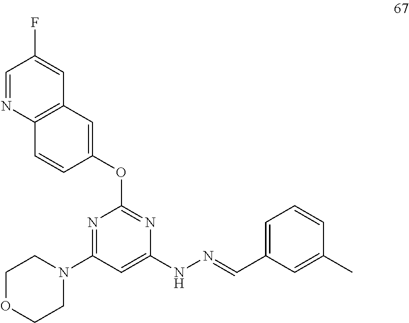 [SMILES: Cc1cccc(/C=N/Nc2cc(N3CCOCC3)nc(Oc3ccc4ncc(F)cc4c3)n2)c1]

Compound 67 was prepared in a similar manner as outlined in Example 1 by using 3-fluoroquinolin-6-ol in step 1. MS-ESI: m/z 459.13 observed (M+H)⁺.

### Example 60: (E)-4-(6-(2-(3-Methylbenzylidene)hydrazinyl)-2-(4-methylquinolin-6-yloxy)pyrimidin-4-yl)morpholine (68)

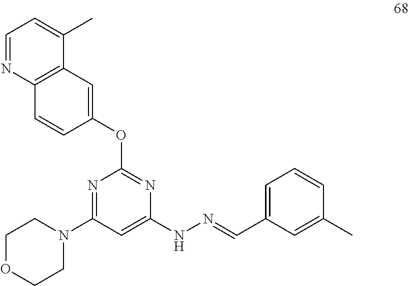 [SMILES: Cc1cccc(/C=N/Nc2cc(N3CCOCC3)nc(Oc3ccc4nccc(C)c4c3)n2)c1]

Compound 68 was prepared in a similar manner as outlined in Example 1 by using 4-methylquinolin-6-ol in step 1. MS-ESI: m/z 455.14 observed (M+H)⁺.

### Example 61: In Vitro Assay

HeLa on 384 well plates with High Glucose DMEM+10% fetal bovine serum (complete medium) 4000 cells/well for 384 well plates were dispensed. The volume of medium per well was kept at 25 μL. Prior to infection, cells were grown to >75% but <100% confluence in a humidified incubator at 37° C.+2° C. with 5% CO₂. Test compounds were added to each assay well at designated concentrations (for 50 μL assay volume) and incubated at 37° C. for 1 hour.

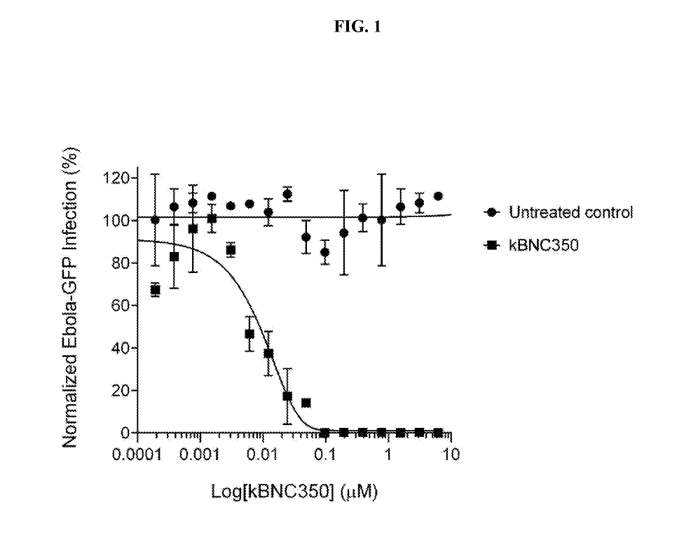

To each well of the assay plate was added 25 μL Ebola viral suspension (GFP-expressing Zaire Ebolavirus Mayinga-GFP) and incubated at 37° C. for 24 hours. The plates were removed from the incubator and inactivated by immersing in 10% neutral buffered formalin. After packaging the plates in a heat sealed bag, the containers were filled with enough 10% formalin to cover the plates. The sealed containers were stored in a 4° C.+5° C. refrigerator overnight. The plates were washed with PBS and stained with Hoechst at room temperature for 30 minutes and then washed with PBS. The GFP and nuclei were imaged using microscope and quantified for number of infected cells and total cells. Biological activity (EC₅₀) for the compounds tested are shown in Table 1. The EC₅₀ activity is graded as: +++=<1 μM; ++=1 μM to 50 μM; and +=>50 μM (NT=not tested). The dose response curve for kBNC350 (Compound 11) is shown in FIG. 1.

TABLE 1

| Cmpd # | Activity |
|---|---|
| 5 | +++ |
| 10 | +++ |
| 11 | +++ |
| 12 | + |
| 13 | +++ |
| 14 | +++ |
| 15 | +++ |
| 16 | + |
| 17 | + |
| 18 | +++ |
| 19 | + |
| 20 | ++ |
| 21 | + |
| 22 | ++ |
| 23 | +++ |
| 24 | + |
| 25 | +++ |
| 26 | ++ |
| 27 | ++ |
| 28 | +++ |
| 29 | ++ |
| 30 | ++ |
| 31 | ++ |
| 32 | ++ |
| 33 | +++ |
| 34 | +++ |
| 35 | ++ |
| 36 | ++ |
| 37 | ++ |
| 38 | + |
| 39 | + |
| 40 | + |
| 41 | + |
| 42 | + |
| 43 | + |
| 44 | ++ |
| 45 | ++ |
| 46 | ++ |
| 47 | +++ |
| 48 | ++ |
| 49 | +++ |
| 50 | +++ |
| 51 | +++ |
| 52 | +++ |
| 53 | +++ |
| 54 | +++ |
| 55 | +++ |
| 56 | +++ |
| 57 | + |
| 58 | +++ |
| 59 | +++ |
| 60 | +++ |
| 61 | + |
| 62 | +++ |
| 63 | +++ |
| 64 | +++ |
| 65 | +++ |
| 66 | +++ |
| 67 | +++ |
| 68 | +++ |

### Example 62: A Study to Evaluate the Effect of a Compound of Formula (I) or (II) in the Treatment of Patients with Ebola Virus Disease (EVD)

The purpose of this study is to assess the efficacy of a compound of Formula (I) or (II) in reducing mortality in humans with EVD.

| Condition | Intervention | Phase |
|---|---|---|
| Ebola Virus | Drug: Compound of | Phase 2 |
| Disease | Formula (I) or (II) | |

Study Type: Interventional

### Study Design

Endpoint Classification: Efficacy Study

Intervention Model: Single Group Assignment

Masking: Open Label

Primary Purpose: Treatment Primary Outcome Measures

The primary outcome will be mortality [Time Frame: Day-14]

[Designated as safety issue: No]

### Secondary Outcome Measures

Evolution of EBOV plasma RNA and infectious loads [Time Frame: routine care venepuncture (Day-0; end of symptoms (EOS)+72 h and EOS+96 h if EOS>Day-9; or Day-12 and Day-13 if EOS<Day-9); (ii) additional trial venepuncture at: Day-2, Day-4 and Day-30 in group A1; Day-2 and Day-30 in group A2] [Designated as safety issue: No]

Occurrence of grade 3 or 4 clinical or biological adverse events (Common Terminology Criteria for Adverse Events, CTAE, v3.0) [Time Frame: participants will be followed for the duration of hospital stay up to Day-14] [Designated as safety issue: Yes]

Evolution of viral micro-diversity of EBOV (including potential resistance mutations)

[Time Frame: routine care venepuncture (Day-0; end of symptoms (EOS)+72 h and EOS+96 h if EOS>Day-9; or Day-12 and Day-13 if EOS<Day-9); (ii) additional trial venepuncture at: Day-2, Day-4 and Day-30 in group A1; Day-2 and Day-30 in group A2]
[Designated as safety issue: No]

Plasma trough concentrations of compound of Formula (I) or (II)

[Time Frame: routine care venepuncture (Day-0; end of symptoms (EOS)+72 h and EOS+96 h if EOS>Day-9; or Day-12 and Day-13 if EOS<Day-9); (ii) additional trial venepuncture at: Day-2, Day-4 and Day-30 in group A1; Day-2 and Day-30 in group A2]
[Designated as safety issue: No]

Criteria for cure [Time Frame: Day-30] [Designated as safety issue: No]

| Arms | Assigned Interventions |
|---|---|
| Experimental: 001 | Drug: Compound of Formula (I) or (II) |
| Compound of Formula | Group A1: Day-0 (inclusion), h 0: 2400 mg; |
| (I) or (II) | h 8: 2400 mg; h 16: 1200 mg. Day-1 to Day-9: |
| (oral administration) | 1200 mg bid. |
| | Group A2: Day-0 (inclusion), h 0: 2400 mg; |
| | h 8: 2400 mg; h 16: 1200 mg. Day-1 to Day-9: |
| | 1200 mg bid. |
| | Group C: daily dosages will be adapted |
| | to their body weight. |

Composite criteria for cure are the following:
4 days without fever or significant symptoms AND;
able to feed and walk independently AND;
two consecutive negative qualitative PCR.

Eligibility

Ages Eligible for Study: 1 year or older

Genders Eligible for Study: Both

Accepts Healthy Volunteers: No

Inclusion Criteria:
age >1 year and weighting >10 kg;
EVD confirmed by a positive qualitative PCR test;
signed informed consent (signed by the parents/adults guardians in case of minor patient).

Exclusion Criteria:
Pregnancy*;
Inability to take the drug (encephalopathy, severe vomiting).
   Emergency use of compound of Formula (I) or (II) in pregnant women outside of the trial is envisaged and under evaluation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating a viral infection comprising administering to an individual in need thereof a therapeutically effective amount of a compound of Formula (II) having the structure: 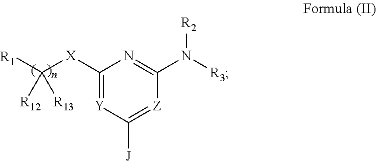
wherein:
X is a bond, —CH₂—, —O—, —S—, —N(R₅)—, —C(═O)—, —S(═O)—, —S(═O)₂—, —C(═N—R₅)—, —C(═N—OR₅)—, or —C(═N—SR₅)—;
one of Y and Z is N, and the other is CR₇;
J is 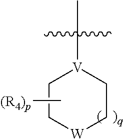
or —N(R₅)(R₆);
V is —C(H)— or —N—;
W is —O—, —S—, —C(R₆)₂—, —N(R₆)—, —N(R₆)C(═O)—, —S(═O)—, or —S(═O)₂—;
R₁, R₂, R₅, and R₆ are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;
R₃ is 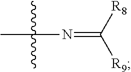
R₄ is substituted or unsubstituted alkyl;
R₇ is H, —CN, halogen, substituted or unsubstituted alkyl, substituted or unsubstituted alkoxy, substituted or unsubstituted cycloalkyl, substituted or unsubstituted heterocycloalkyl, substituted or unsubstituted aryl, or substituted or unsubstituted heteroaryl;
R₈ and R₉ are each independently H, substituted or unsubstituted alkyl, substituted or unsubstituted aryl, or substituted or unsubstituted heteroaryl;
each R₁₂ and R₁₃ are each independently H, halogen, or substituted or unsubstituted alkyl;
n is 0, 1, 2, 3, 4, 5, or 6;
p is 0, 1, 2, 3, 4, 5, or 6; and
q is 1 or 2;
or a pharmaceutically acceptable salt, solvate, or prodrug thereof.

2. The method of claim 1, wherein R₂ is H.

3. The method of claim 1 or claim 2, wherein J is q

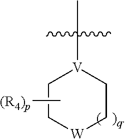

4. The method of claim 3, wherein p is 0 and q is 1.

5. The method of any one of claims 1-4, wherein R₈ is H.

6. The method of any one of claims 1-5, wherein R₉ is substituted aryl.

7. The method of any one of claims 1-6, wherein R₉ is

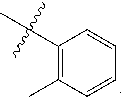 [SMILES: *c1ccccc1C]

8. The method of any one of claims 1-6, wherein R₉ is

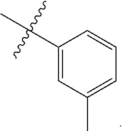 [SMILES: *c1cccc(C)c1]

9. The method of any one of claims 1-6, wherein R₉ is

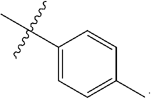 [SMILES: *c1ccc(C)cc1]

10. The method of any one of claims 1-4, wherein R₈ is H or substituted or unsubstituted alkyl, and R₉ is 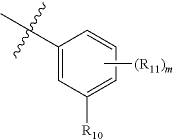
wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4.

11. The method of claim 10, wherein R₁₀ is alkyl and m is 0.

12. The method of claim 11, wherein R₈ is H.

13. The method of any one of claims 1-12, wherein X is —O—.

14. The method of any one of claims 1-13, wherein Y is N; and Z is CR₇.

15. The method of any one of claims 1-14 wherein R₇ is H.

16. The method of any one of claims 1-15, wherein V is —N—.

17. The method of any one of claims 1-16, wherein W is —O—.

18. The method of any one of claims 1-17, wherein R₁ is substituted or unsubstituted alkyl, substituted or unsubstituted cycloalkyl, substituted or unsubstituted heterocycloalkyl, substituted or unsubstituted aryl, or substituted or unsubstituted heteroaryl.

19. The method of any one of claims 1-17, wherein R₁ is substituted or unsubstituted heterocycloalkyl, substituted or unsubstituted aryl, or substituted or unsubstituted heteroaryl.

20. The method of any one of claims 1-17, wherein R₁ is substituted or unsubstituted heteroaryl.

21. The method of any one of claims 1-17, wherein R₁ is substituted or unsubstituted quinolinyl.

22. The method of any one of claims 1-17, wherein R₁ is substituted or unsubstituted pyridyl.

23. The method of any one of claims 1-17, wherein R₁ is

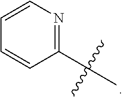 [SMILES: *c1ccccn1]

24. The method of any one of claims 1-23, wherein each R₁₂ and R₁₃ are H.

25. The method of any one of claims 1-24, wherein n is 0, 1, 2, or 3.

26. The method of any one of claims 1-23, wherein n is 0.

27. The method of any one of claims 1-24, wherein n is 1.

28. The method of any one of claims 1-24, wherein n is 2.

29. The method of any one of claims 1-24, wherein n is 3.

30. A method for treating a viral infection comprising administering to an individual in need thereof a therapeutically effective amount of a compound of Formula (I) having the structure: 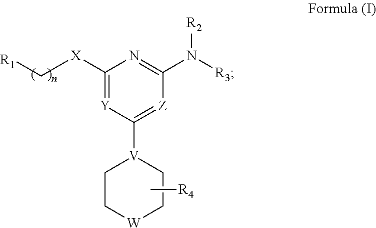
wherein:
X is a bond, —CH₂—, —O—, —S—, —N(R₅)—, —C(═O)—, —S(═O)—, —S(═O)₂—, —C(═N—R₅)—, —C(═N—OR₅)—, or —C(═N—SR₅)—;
one of Y and Z is N, and the other is CR₇;
V is —C(H)— or —N—;
W is —O—, —S—, —N(R₆)—, —N(R₆)C(═O)—, —S(═O)—, or —S(═O)₂—;
R₁, R₂, R₅, R₆, and R₇ are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;
R₃ is 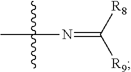
R₄ is H or alkyl;
R₈ and R₉ are each independently H, substituted or unsubstituted alkyl, substituted or unsubstituted aryl, or substituted or unsubstituted heteroaryl; and
n is 0, 1, 2, 3, 4, 5, or 6;
or a pharmaceutically acceptable salt, solvate, or prodrug thereof.

31. The method of claim 30, wherein R₂ is H.

32. The method of claim 30 or claim 31, wherein R₄ is H.

33. The method of any one of claims 30-32, wherein R₈ is H.

34. The method of any one of claims 30-33, wherein R₉ is substituted aryl.

35. The method of any one of claims 30-34, wherein R₉ is

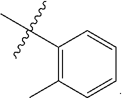 [SMILES: *c1ccccc1C]

36. The method of any one of claims 30-34, wherein R₉ is

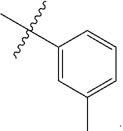 [SMILES: *c1cccc(C)c1]

37. The method of any one of claims 30-34, wherein R₉ is

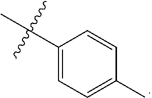 [SMILES: *c1ccc(C)cc1]

38. The method of any one of claims 30-32, wherein R₈ is H or substituted or unsubstituted alkyl, and R₉ is 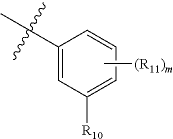
wherein R₁₀ is H, halogen, alkyl, or alkoxy; R₁₁ is halogen, —CN, —OH, alkyl, aryl, heteroaryl, alkoxy, aryloxy, or heteroaryloxy; and m is 0, 1, 2, 3, or 4.

39. The method of claim 38, wherein R₁₀ is alkyl and m is 0.

40. The method of claim 39, wherein R₈ is H.

41. The method of any one of claims 30-40, wherein X is —O—.

42. The method of any one of claims 30-41, wherein Y is N; and Z is CR₇.

43. The method of claim 42 wherein R₇ is H.

44. The method of any one of claims 30-43, wherein V is —N—.

45. The method of any one of claims 30-44, wherein W is —O—.

46. The method of any one of claims 30-45, wherein R₁ is substituted or unsubstituted alkyl, substituted or unsubstituted cycloalkyl, substituted or unsubstituted heterocycloalkyl, substituted or unsubstituted aryl, or substituted or unsubstituted heteroaryl.

47. The method of any one of claims 30-45, wherein R₁ is substituted or unsubstituted heterocycloalkyl, substituted or unsubstituted aryl, or substituted or unsubstituted heteroaryl.

48. The method of any one of claims 30-45, wherein R₁ is substituted or unsubstituted heteroaryl.

49. The method of any one of claims 30-45, wherein R₁ is substituted or unsubstituted quinolinyl.

50. The method of any one of claims 30-45, wherein R₁ is substituted or unsubstituted pyridyl.

51. The method of any one of claims 30-45, wherein R₁ is

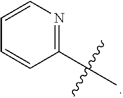 [SMILES: *c1ccccn1]

52. The method of any one of claims 30-51, wherein n is 0, 1, 2, or 3.

53. The method of any one of claims 30-51, wherein n is 0.

54. The method of any one of claims 30-51, wherein n is 1.

55. The method of any one of claims 30-51, wherein n is 2, 3, 4, 5, or 6.

56. The method of any one of claims 30-51, wherein n is 2.

57. The method of claim 1 or 30, wherein the compound of Formula (I) or Formula (II) is:

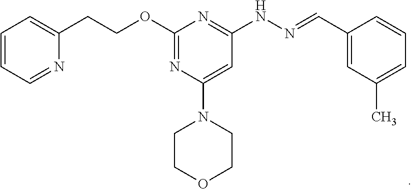 [SMILES: Cc1cccc(/C=N/Nc2cc(N3CCOCC3)nc(OCCc3ccccn3)n2)c1]

58. The method of any one of claims 1-57, wherein the viral infection is from an RNA virus.

59. The method of any one of claims 1-58, wherein the viral infection is from the Ebola virus.

60. The method of any one of claims 1-59, further comprising administration of a second agent.

61. The method of claim 60, further comprising administration of a second agent selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, and T-705.

62. The method of claim 60, further comprising administration of a second agent selected from Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, T-705, and GS-5734.

63. A compound having the structure 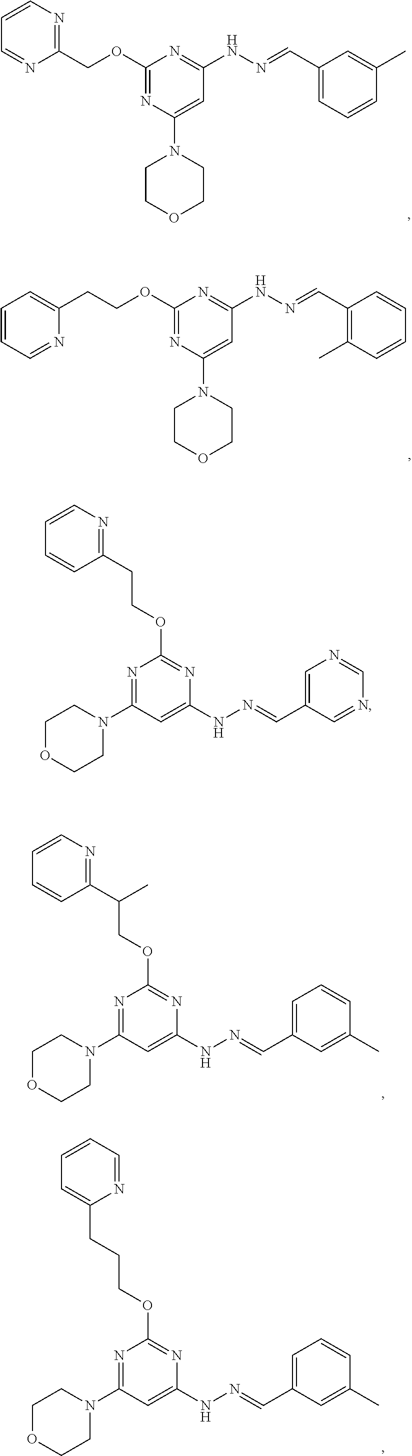 [SMILES: Cc1cccc(/C=N/Nc2cc(N3CCOCC3)nc(OCc3ncccn3)n2)c1 ; Cc1ccccc1/C=N/Nc1cc(N2CCOCC2)nc(OCCc2ccccn2)n1 ; C(=N\Nc1cc(N2CCOCC2)nc(OCCc2ccccn2)n1)/c1cncnc1 ; Cc1cccc(/C=N/Nc2cc(N3CCOCC3)nc(OCC(C)c3ccccn3)n2)c1 ; Cc1cccc(/C=N/Nc2cc(N3CCOCC3)nc(OCCCc3ccccn3)n2)c1] 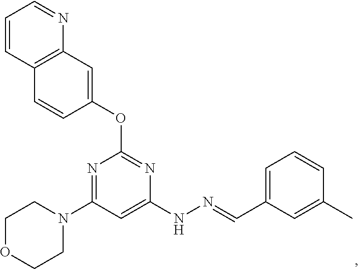 [SMILES: Cc1cccc(/C=N/Nc2cc(N3CCOCC3)nc(Oc3ccc4cccnc4c3)n2)c1] 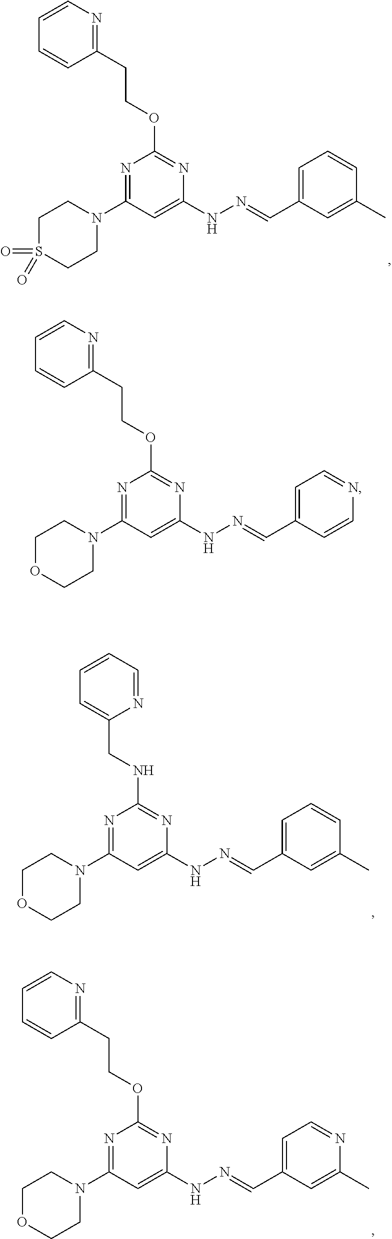 [SMILES: Cc1cccc(/C=N/Nc2cc(N3CCS(=O)(=O)CC3)nc(OCCc3ccccn3)n2)c1 ; C(=N\Nc1cc(N2CCOCC2)nc(OCCc2ccccn2)n1)/c1ccncc1 ; Cc1cccc(/C=N/Nc2cc(N3CCOCC3)nc(NCc3ccccn3)n2)c1 ; Cc1cc(/C=N/Nc2cc(N3CCOCC3)nc(OCCc3ccccn3)n2)ccn1] 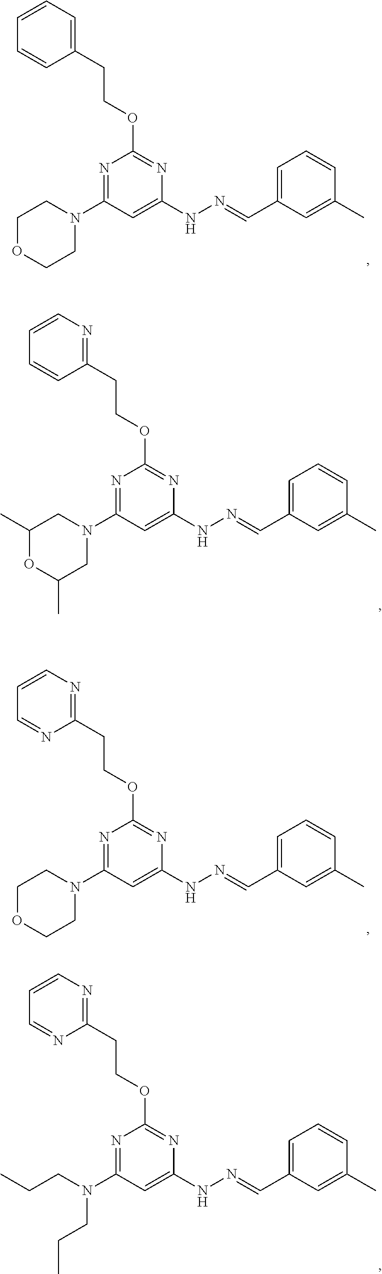 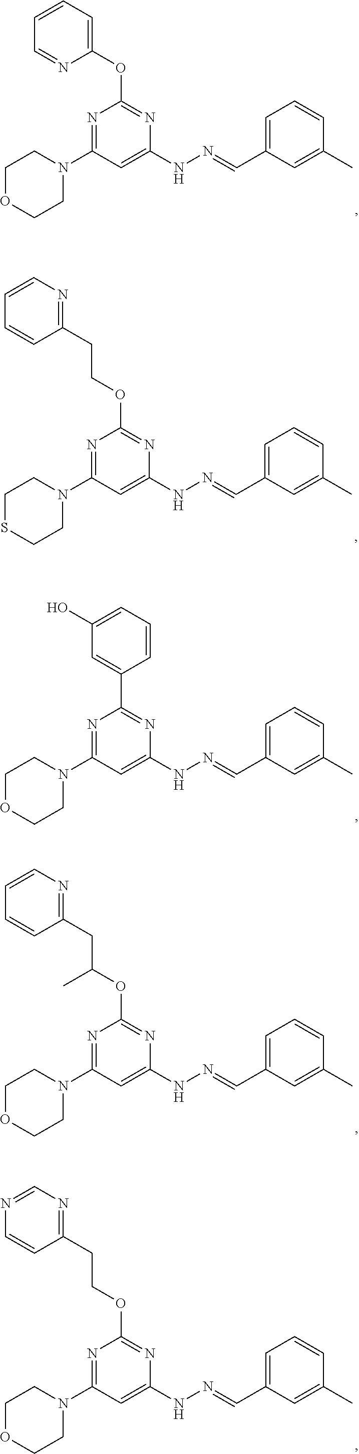 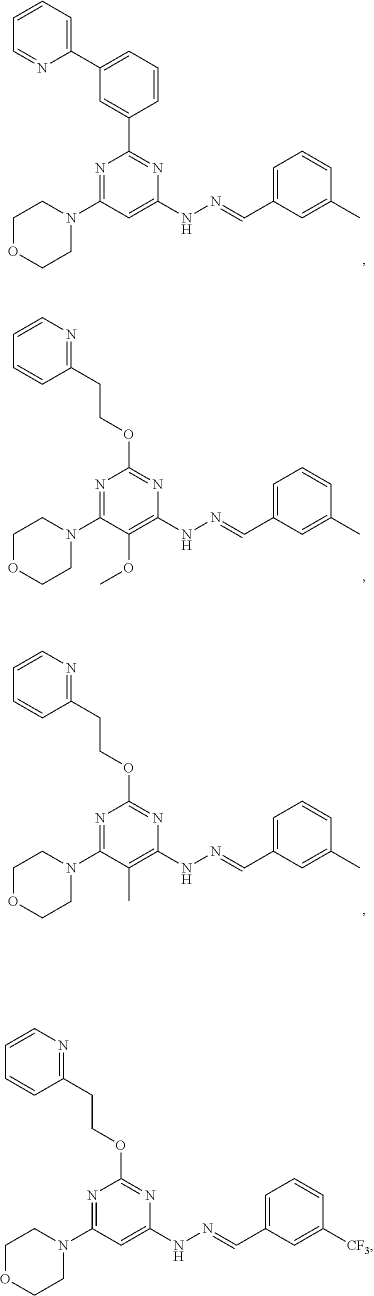 [SMILES: Cc1cccc(/C=N/Nc2cc(N3CCOCC3)nc(-c3cccc(-c4ccccn4)c3)n2)c1 ; COc1c(N/N=C/c2cccc(C)c2)nc(OCCc2ccccn2)nc1N1CCOCC1 ; Cc1cccc(/C=N/Nc2nc(OCCc3ccccn3)nc(N3CCOCC3)c2C)c1 ; FC(F)(F)c1cccc(/C=N/Nc2cc(N3CCOCC3)nc(OCCc3ccccn3)n2)c1] 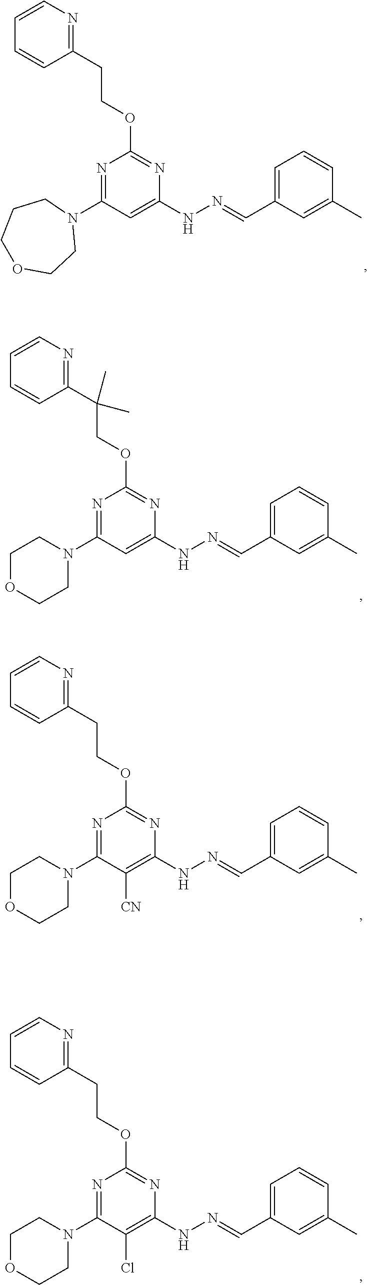 [SMILES: Cc1cccc(/C=N/Nc2cc(N3CCCOCC3)nc(OCCc3ccccn3)n2)c1 ; Cc1cccc(/C=N/Nc2cc(N3CCOCC3)nc(OCC(C)(C)c3ccccn3)n2)c1 ; Cc1cccc(/C=N/Nc2nc(OCCc3ccccn3)nc(N3CCOCC3)c2C#N)c1 ; Cc1cccc(/C=N/Nc2nc(OCCc3ccccn3)nc(N3CCOCC3)c2Cl)c1] 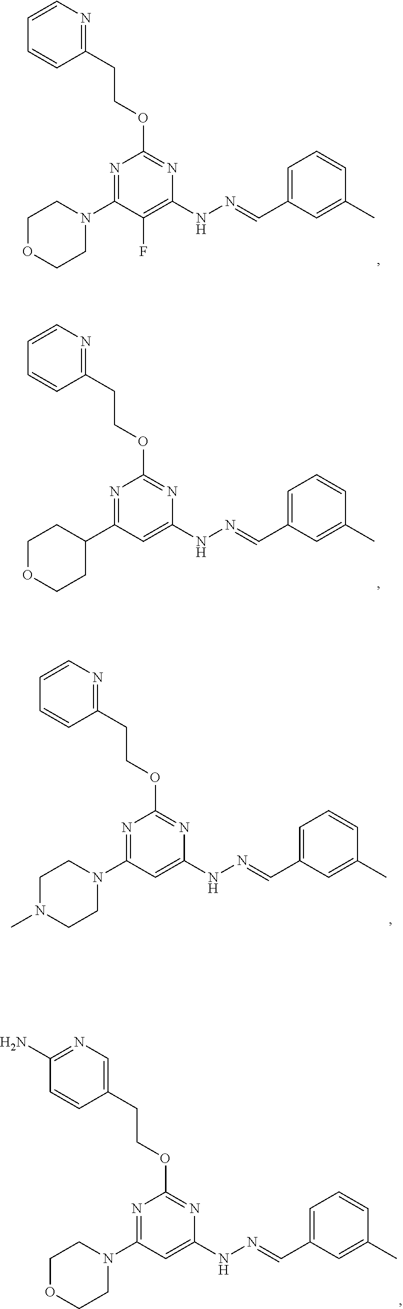 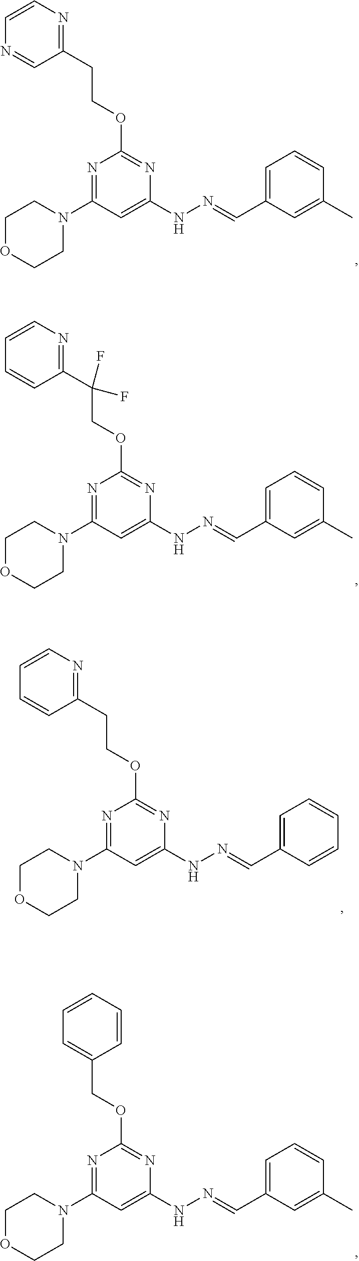 [SMILES: Cc1cccc(/C=N/Nc2cc(N3CCOCC3)nc(OCCc3cnccn3)n2)c1 ; Cc1cccc(/C=N/Nc2cc(N3CCOCC3)nc(OCC(F)(F)c3ccccn3)n2)c1 ; C(=N\Nc1cc(N2CCOCC2)nc(OCCc2ccccn2)n1)/c1ccccc1 ; Cc1cccc(/C=N/Nc2cc(N3CCOCC3)nc(OCc3ccccc3)n2)c1] 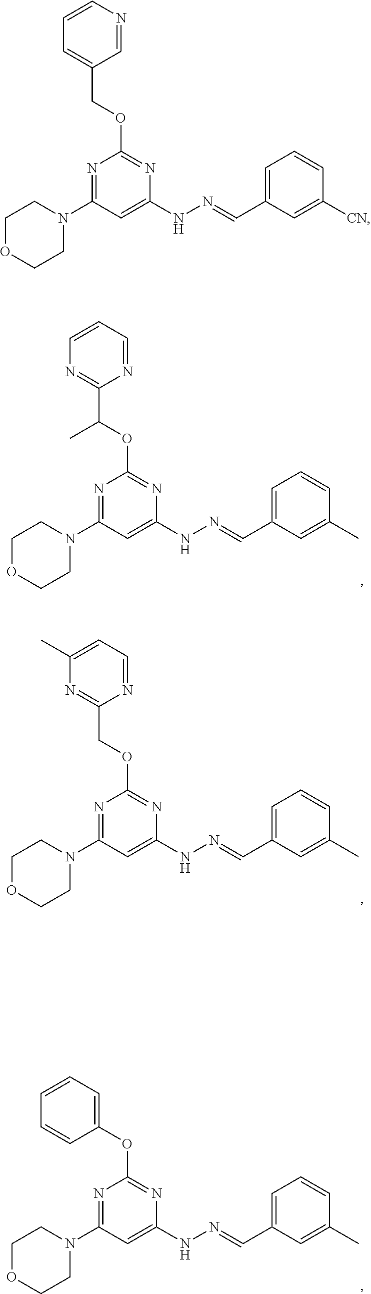 [SMILES: N#Cc1cccc(/C=N/Nc2cc(N3CCOCC3)nc(OCc3cccnc3)n2)c1 ; Cc1cccc(/C=N/Nc2cc(N3CCOCC3)nc(OC(C)c3ncccn3)n2)c1 ; Cc1cccc(/C=N/Nc2cc(N3CCOCC3)nc(OCc3nccc(C)n3)n2)c1 ; Cc1cccc(/C=N/Nc2cc(N3CCOCC3)nc(Oc3ccccc3)n2)c1] 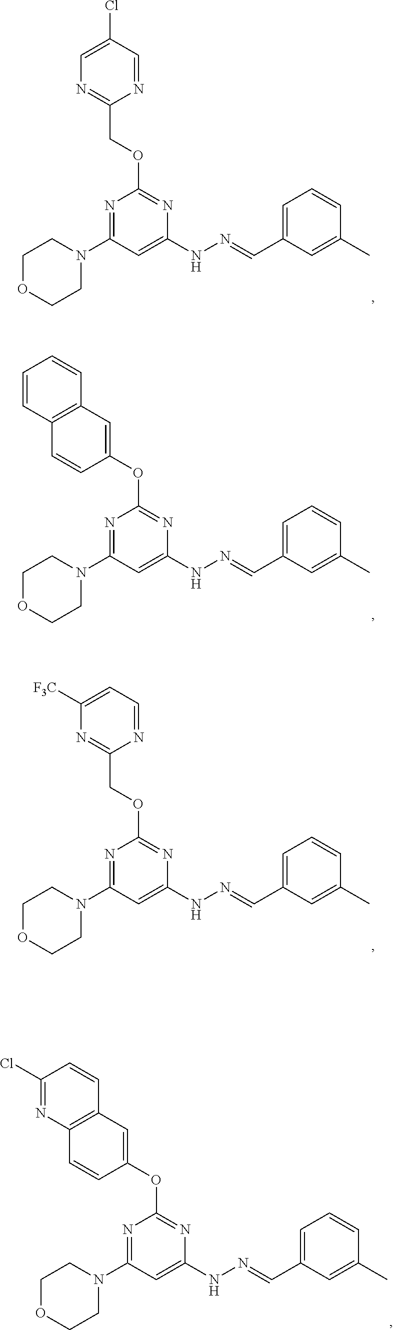 [SMILES: Cc1cccc(/C=N/Nc2cc(N3CCOCC3)nc(OCc3ncc(Cl)cn3)n2)c1 ; Cc1cccc(/C=N/Nc2cc(N3CCOCC3)nc(Oc3ccc4ccccc4c3)n2)c1 ; Cc1cccc(/C=N/Nc2cc(N3CCOCC3)nc(OCc3nccc(C(F)(F)F)n3)n2)c1 ; Cc1cccc(/C=N/Nc2cc(N3CCOCC3)nc(Oc3ccc4nc(Cl)ccc4c3)n2)c1] 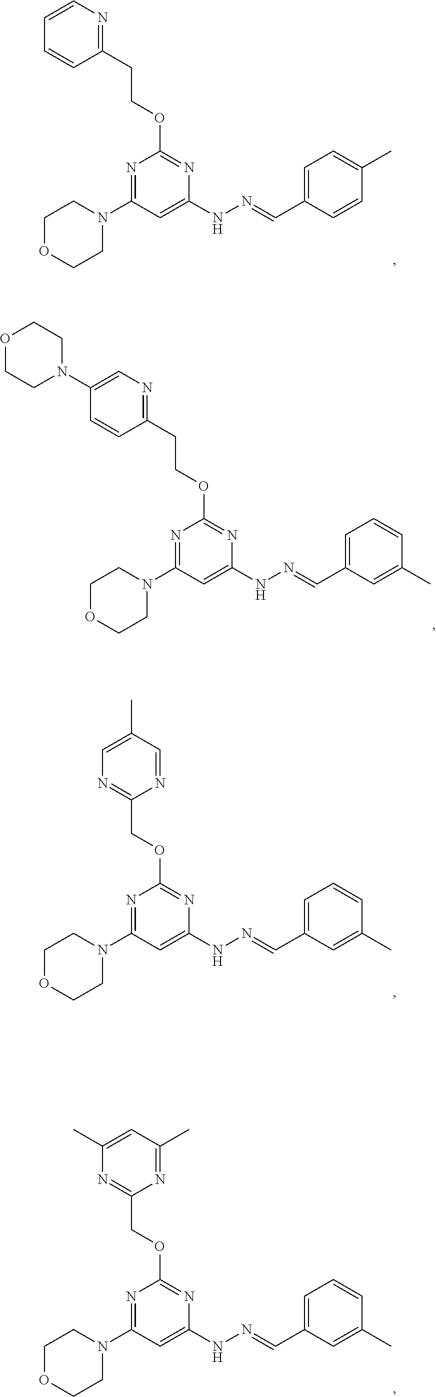 [SMILES: Cc1ccc(/C=N/Nc2cc(N3CCOCC3)nc(OCCc3ccccn3)n2)cc1 ; Cc1cccc(/C=N/Nc2cc(N3CCOCC3)nc(OCCc3ccc(N4CCOCC4)cn3)n2)c1 ; Cc1cnc(COc2nc(N/N=C/c3cccc(C)c3)cc(N3CCOCC3)n2)nc1 ; Cc1cccc(/C=N/Nc2cc(N3CCOCC3)nc(OCc3nc(C)cc(C)n3)n2)c1] 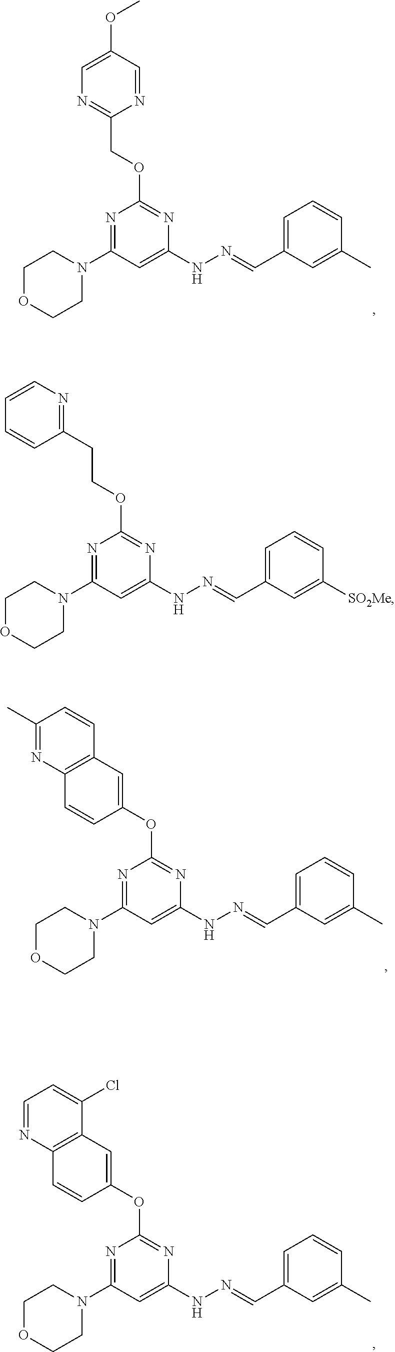 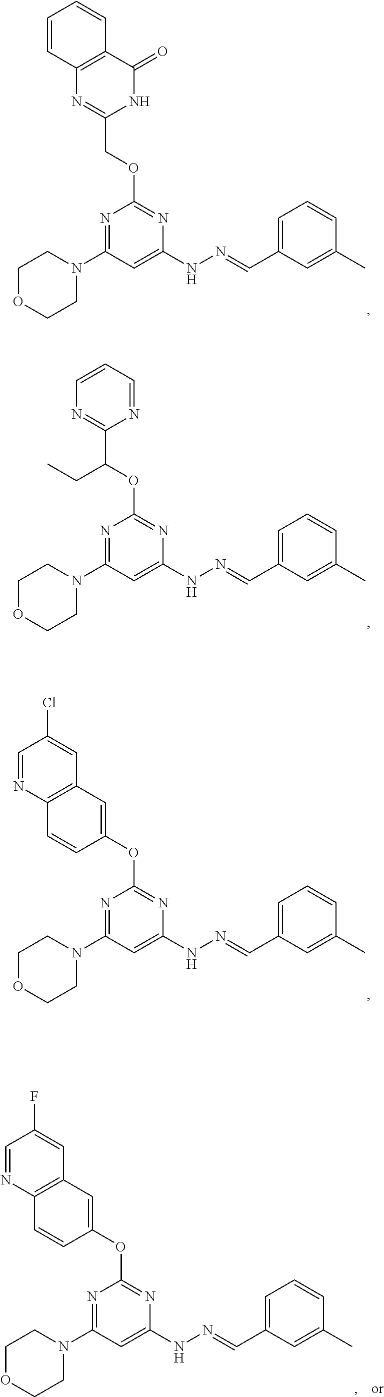 [SMILES: Cc1cccc(/C=N/Nc2cc(N3CCOCC3)nc(OCc3nc4ccccc4c(=O)[nH]3)n2)c1 ; CCC(Oc1nc(N/N=C/c2cccc(C)c2)cc(N2CCOCC2)n1)c1ncccn1 ; Cc1cccc(/C=N/Nc2cc(N3CCOCC3)nc(Oc3ccc4ncc(Cl)cc4c3)n2)c1 ; Cc1cccc(/C=N/Nc2cc(N3CCOCC3)nc(Oc3ccc4ncc(F)cc4c3)n2)c1] 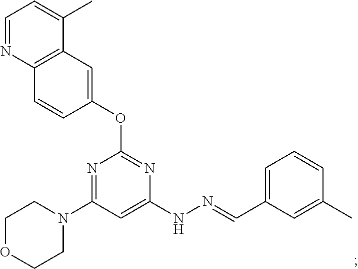 [SMILES: Cc1cccc(/C=N/Nc2cc(N3CCOCC3)nc(Oc3ccc4nccc(C)c4c3)n2)c1]
or a pharmaceutically acceptable salt, solvate, or prodrug thereof.

64. A pharmaceutical composition comprising a compound of claim 63, or a pharmaceutically acceptable salt, solvate, or prodrug thereof, and a pharmaceutically acceptable excipient.
